SEQUENCE LISTING (6 sequences)

SEQ ID NO: 1 - RNA, 21 nt, synthetic construct
  source 1..21: mol_type = other RNA; note = Sense sequence
  modified_base 20..21: mod_base = OTHER; note = thymine
gttctcagccattgctagctt

SEQ ID NO: 2 - RNA, 21 nt, synthetic construct
  source 1..21: mol_type = other RNA; note = Antisense sequence
  modified_base 20..21: mod_base = OTHER; note = thymine
gctagcaatggctgagaactt

SEQ ID NO: 3 - RNA, 21 nt, synthetic construct
  source 1..21: mol_type = other RNA; note = Sense sequence
  modified_base 20..21: mod_base = OTHER; note = thymine
ccacttgtgatgacatctttt

SEQ ID NO: 4 - RNA, 21 nt, synthetic construct
  source 1..21: mol_type = other RNA; note = Antisense sequence
  modified_base 20..21: mod_base = OTHER; note = thymine
aagatgtcatcacaagtggtt

SEQ ID NO: 5 - RNA, 21 nt, synthetic construct
  source 1..21: mol_type = other RNA; note = Sense sequence
  modified_base 20..21: mod_base = OTHER; note = thymine
ttctccgaacgtgtcacgttt

SEQ ID NO: 6 - RNA, 21 nt, synthetic construct
  source 1..21: mol_type = other RNA; note = Antisense sequence
  modified_base 20..21: mod_base = OTHER; note = thymine
acgtgacacgttcggagaatt

(19) United States
(12) Patent Application Publication
(10) Pub. No.: US 2026/0009085 A1
(43) Pub. Date: Jan. 8, 2026

(54) METHODS AND COMPOSITIONS FOR THE DIAGNOSIS AND TREATMENT OF CANCER

(71) Applicant: Chengdu Anticancer Bioscience, Ltd., Chengdu (CN)

(72) Inventors: Dun Yang, Chengdu (CN); Qiong Shi, Chengdu (CN); Ting Zhang, Chengdu (CN); Iuliia Kalashova, Chengdu (CN); Gang Lv, Chengdu (CN); Chenglu Yang, Chengdu (CN); Hongmei Li, Chengdu (CN); Xiaohu Zhou, Chengdu (CN); Yan Long, Chengdu (CN); Shenqiu Zhang, St. Andrews (GB); Hong Liu, El Sobrante, CA (US); Thaddeus Allen, Daly City, CA (US); Jing Zhang, Chengdu (CN)

(21) Appl. No.: 19/324,882

(22) Filed: Sep. 10, 2025

Related U.S. Application Data
(63) Continuation of application No. PCT/CN2024/083981, filed on Mar. 27, 2024.

(30) Foreign Application Priority Data
Mar. 28, 2023 (WO) PCT/CN2023/084430

(51) Int. Cl. C12Q 1/6886; G01N 33/50

(52) U.S. Cl. CPC C12Q 1/6886; G01N 33/5011; C12Q 2600/118; C12Q 2600/136; C12Q 2600/158

(57) ABSTRACT

The present invention relates to the determination of expression or activity levels of particular biomarkers in biological samples which can be utilized to diagnose, prognose, and treat cancer in individuals, and further to select individuals who would benefit from a cancer therapy such as treatment with a one or more agent(s) that inhibit cancer cell viability and/or proliferation. Accordingly, the present invention encompasses methods that utilize these biomarkers for the diagnosis, prognosis, and treatment of cancer as well as screening for compounds that reduce the risk of an individual developing cancer, reduce the risk of an individual developing one or more symptoms of cancer, and alleviate one or more symptoms of cancer in an individual.

### RELATED APPLICATIONS

This application is a continuation of International Patent Application No. PCT/CN2024/083981, filed Mar. 27, 2024, which claims the benefit of and priority to Patent Application No. PCT/CN2023/084430, filed Mar. 28, 2023, each of which are incorporated herein by reference in their entireties for all purposes.

### SEQUENCE LISTING

The instant application contains a Sequence Listing which has been submitted electronically and is hereby incorporated by reference in its entirety. Said XML copy, created on Jul. 29, 2025, is named F24W8270-SL.xml, and is 10,491 bytes in size.

### FIELD OF THE INVENTION

Provided herein are diagnostic and therapeutic methods for the treatment of cancer using one or more agent(s) that inhibit cancer cell viability and/or proliferation response.

### BACKGROUND

Cell division is a highly complex and coordinated process controlled by upstream signaling pathways. Any therapeutic that targets this process may be a valuable tool to attack cancer cells. For example, two classes of mitotic spindle targeting agent, taxanes and vinca alkaloids, have been used clinically for decades. However, due to mechanism-associated side effects, such as neurotoxicity, long-term patient use of these spindle toxins is challenging. In addition, drug resistance can make their use limiting. A novel anti-mitotic that overcomes these limitations would be a valuable tool for treating cancer.

One way to develop next-generation mitotic inhibitors may be to attack targets other than microtubules, as this approach might negate unwanted toxic effects in normal tissues. Populations of normal dividing cells, required for tissue homeostasis, might be spared by targeting a mitotic vulnerability specifically enabled by oncogenic transformation. This would enable the selective killing of tumor cells as opposed to normal dividing cells. This kind of synthetic lethal (SL) drug interaction arises when the genetic and epigenetic alterations that promote carcinogenesis also leave cancer cells highly dependent on specific cellular proteins and pathways for survival. Therapeutic targeting of these dependencies can produce a potent synthetic lethal effect.

Since oncogenic alterations vary among cancer cells, there is not just a need to find novel antimitotic agents, one must also be able to identify the vulnerabilities these new agents act upon and determine which tumors harbor vulnerability. Biomarkers that pinpoint patient populations susceptible to synthetic lethal therapy are required for the success of antimitotic agents.

Deregulation of the MYC proto-oncogene represents one of the most frequent anomalies in human malignancies and correlates with poorly differentiated, very aggressive, and difficult-to-treat cancer. Although generally considered an important and broadly applicable oncology target, MYC encodes a transcription factor that has proven difficult to inhibit with small molecules. Furthermore, systemic inhibition of MYC may have undesirable consequences because MYC has many targets and the long-term effects of its inhibition are not known. To optimize long-term use, a therapy that directly inhibits MYC may require persistent fine-tuning of the dose and duration of treatment.

Overexpression of MYC promotes various mitotic abnormalities and renders tumor cells particularly susceptible to inhibition of mitotic Aurora kinases (AURKs) and the microtubule nucleation factor TPX2 (Rohrberg, Julia, et al. Cell reports 30.10 (2020): 3368-3382; 2020; Takahashi, Y., et al. Annals of oncology 26.5 (2015): 935-942). Targeting Aurora kinases (AURKs) or TPX2 in MYC overexpressing cells represent examples of synthetic lethal approaches to attack the mitotic machinery. Unlike taxane and vinca alkaloid attack, anticancer activity by targeting AURKs or TPX2 is not dependent on direct targeting of the microtubular spindle apparatus. These synthetic lethal approaches open the door to the development of alternative approaches to attacking cells overexpressing the MYC oncogene. Such an approach would also have the potential to bypass liabilities associated with inhibition of MYC or the mitotic spindle in all cells.

The three AURKs, (AURKA, AURKB, and AURKC), comprise a family of serine/threonine kinases that are vital for proper cell division. AURKC is expressed specifically in the mammalian testis and plays a role in meiosis. AURKA and AURKB are found in all metazoan cells. AURKA becomes localized during mitosis to the spindle poles where it phosphorylates targets essential for the maturation and function of centrosomes during assembly of the bipolar mitotic spindle. AURKB, in contrast, is the catalytic subunit of the chromosomal passenger protein complex (CPPC). The CPPC also includes a scaffold protein called Inner Centromere Protein (INCENP), and two small regulatory components, Survivin (also known as BIRC5) and Borealin (also known as CDCA8). The CPPC complex is a master coordinator of karyokinesis and cytokinesis. Execution of its diverse mitotic functions requires dynamic, highly coordinated relocation of the CPPC complex to specific mitotic structures. For example, during early mitosis, the CPPC complex is enriched at centromeres and regulates kinetochore function, the fidelity of sister chromatid separation, and the spindle assembly checkpoint. Upon transition from metaphase to anaphase, the CPPC complex relocates to the spindle midzone before transport to the equatorial cortex. Signals from the anaphase spindle direct the formation and position of a contractile ring at the cell cortex. The CPPC complex participates in cytokinesis by initiating signaling from the spindle midzone and equatorial cortex. In telophase, the CPPC complex is localized to multiple bands flanking the midbody to direct the completion of cytokinesis. Mitotic kinesin-like protein 2 (MKLP2), also called KIF20A, is a protein known to direct these versatile localization patterns of the CPPC in dividing cells.

MKLP2 is a processive microtubule plus-end-directed motor protein that harnesses the energy generated through ATP hydrolysis to travel along dynamic microtubules. MKLP2 was initially studied as a motor protein required for the retrograde RAB6-regulated transport of Golgi membranes and associated vesicles along microtubules. Later studies revealed an important role for MKLP2 in mitosis.

MKLP2 functions in early mitosis to promote chromosome congregation via correction of syntenic attachments. The protein is also required at the onset of anaphase to promote cytokinesis by facilitating the relocation of the CPPC complex. MKLP2 interacts with the INCENP subunit of the CPPC complex to remove it from chromosomes at the beginning of anaphase. The MKLP2 and the CPPC complex target to the spindle midzone in an interdependent manner. Finally, the CPPC complex is transported by MKLP2 from the midzone to the equatorial cortex. All of these distinct localizations of the CPPC complex require the motor activity of MKLP2.

Published literature on MYC SL, whether with therapeutics or genetic deletion of a synthetic lethal gene, has typically described a synthetic lethal interaction arising from a single screen or assays carried out in a limited number of cell lines. Likewise, the in vivo validation of SL with MYC overexpression is typically confirmed in a MYC overexpression mouse model, such as the Em-MYC transgenic model. One drawback to this approach is the selection for SL that may be adequate in the models assayed but could have very limited efficacy in tumors where MYC is expressed, but is not the direct driver of carcinogenesis. This would lead to limited utility in human tumors where MYC is expressed alongside many other genomic and epigenomic alterations.

### SUMMARY

By screening human cell lines, Applicants have discovered that the expression and activity of particular biomarkers (e.g., one or more genes of the cancer-dependent gene signature (e.g., expression and/or activity of MYC that is elevated relative to a suitable control and/or expression and/or activity of MKLP2 that is decreased relative to a suitable control)) or modification of a MYC-dependent cellular phenotypic signature in biological samples (e.g., tissues, plasma, blood, stool, urine, or combinations thereof) can be utilized to diagnose, prognose, and treat cancer in individuals, and further to select individuals who would benefit from a therapy in which one or more agent(s) that inhibit cancer cell viability and/or proliferation are administered. Demonstrated herein is the correlation of the potency of agents that inhibit cancer cell viability and/or proliferation with MYC and/or MKLP2 expression. Accordingly, the present invention encompasses methods that utilize genes of the cancer-dependent gene signature for the diagnosis, prognosis, and treatment of cancer. The present invention also encompasses a novel assay to screen agents for their inhibition of mitosis (e.g., methods that utilize phenotypes of the MYC-dependent cellular phenotypic signature for the diagnosis, prognosis, and treatment of cancer). The phenotypic assay scores for cellular phenotypes including transient mitotic arrest and an accumulation of multinucleated, polyploid cells. The phenotypic differences observed with distinct classes of screened agents (e.g., potential mitotic inhibitors) enables quick identification of new molecules that may inhibit mitosis through high-throughput screening.

In certain aspects, disclosed herein are methods of treating an individual at risk of developing cancer, suffering from one or more symptoms associated with cancer, and/or diagnosed with cancer, comprising: obtaining a dataset comprising data associated with expression and/or activity of MYC and MKLP2 in a biological sample obtained from the individual; and identifying the individual as at risk of developing cancer or having cancer when the expression and/or activity of MYC is increased and the expression and/or activity of MKLP2 is decreased relative to a suitable control. In certain embodiments, the methods further comprise obtaining the biological sample from the individual, and optionally further comprising processing the sample to produce the dataset. In certain embodiments, the biological sample comprises a tissue, plasma, blood, stool, urine, or combinations thereof. In certain embodiments, the biological sample is obtained from a tissue biopsy, aspirate, or surgical removal. In certain embodiments, the methods further comprise administering to the individual one or more agent(s) that inhibit cancer cell viability and/or proliferation.

In certain aspects, described herein are methods of treating a cancer with increased expression and/or activity of MYC and decreased expression and/or activity of MKLP2 in an individual, the method comprising administering to the individual one more agent(s) that inhibit cancer cell viability and/or proliferation. In certain embodiments, the one or more agent(s) that inhibit cancer cell viability and/or proliferation comprises an agent that dysregulates MYC, one or more mitotic kinase(s), one or more mitotic motor protein(s), and/or an upstream regulator or a downstream effector of the chromosomal passenger protein complex (CPPC). In certain embodiments, the one or more agent(s) that inhibit cancer cell viability and/or proliferation comprises an agent selected from LC30, LW33R, LXY013, LXY018, LC02, LC09, LG157, LG160, LG169, LG171, LG172, LG177, and LG181.

In certain aspects, described herein are methods for identifying an individual who may benefit from a treatment comprising one or more agent(s) that inhibit cancer cell viability and/or proliferation, comprising: obtaining a dataset associated with expression and/or activity of MYC in a biological sample obtained from the individual; wherein an expression and/or activity of MYC that is elevated relative to a suitable control identifies the individual as one who may benefit from a treatment comprising one or more agent(s) that inhibit cancer cell viability and/or proliferation; and wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC. In certain embodiments, the methods further comprise obtaining the biological sample from the individual, and optionally further comprising processing the sample to produce the dataset. In certain embodiments, the agent that inhibits cancer cell viability, comprises an agent selected from LC30, LW33R, LXY013, LXY018, LC02, LC09, LG157, LG160, LG169, LG171, LG172, LG177, and LG181.

In certain aspects, described herein are methods for identifying an individual who may benefit from a treatment comprising one or more agent(s) that inhibit cancer cell viability and/or proliferation, comprising: obtaining a dataset comprising data associated with expression and/or activity of MKLP2 in biological sample obtained from the individual; wherein an expression and/or activity of MKLP2 that is decreased relative to a suitable control identifies the individual as one who may benefit from a treatment comprising one or more agent(s) that inhibit cell viability; and wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC. In certain embodiments, the methods further comprise obtaining the biological sample from the individual, and optionally further comprising processing the sample to produce the dataset. In certain embodiments, the one or more agent(s) that inhibit cancer cell viability and/or proliferation comprises an agent selected from LC30, LW33R, LXY013, LXY018, LC02, LC09, LG157, LG160, LG169, LG171, LG172, LG177, and LG181.

In certain aspects, described herein are methods for identifying an individual who may benefit from a treatment comprising one or more agent(s) that inhibit cancer cell viability and/or proliferation, comprising: obtaining a dataset comprising data associated with expression and/or activity of MYC and MKLP2 in a biological sample obtained from the individual; wherein an expression and/or activity of MYC that is elevated relative to a suitable control and an expression and/or activity of MKLP2 that is decreased relative to a suitable control identifies an individual as one who may benefit from a treatment comprising one or more agent(s) that inhibit cancer cell viability and/or proliferation. In certain embodiments, methods further comprise obtaining the biological sample from the individual, and optionally further comprising processing the sample to produce the dataset. In certain embodiments, the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC. In certain embodiments, the one or more agent(s) that inhibit cancer cell viability and/or proliferation comprises an agent selected from LC30, LW33R, LXY013, LXY018, LC02, LC09, LG157, LG160, LG169, LG171, LG172, LG177, and LG181.

In certain aspects, described herein are methods of screening compounds for the treatment of cancer, comprising: contacting cancer cells or a sample derived from cancer cells with one or more test agents; detecting the expression and/or activity of MYC and MKLP2 in the cancer cells; and if the test agent modifies a cancer-dependent gene signature or MYC-dependent cellular phenotypic signature, identifying the test agent as a compound effective for the treatment of cancer. In certain embodiments, the cancer cells are cancer-derived cells, immortalized cells, or primary cells.

In certain embodiments, the detecting comprises detecting MYC and/or MKLP protein. In certain embodiments, the detecting comprises performing an immunofluorescence assay, an immunoblot, a proteomic analysis, or an ELISA on the biological sample or cancer cells. In certain embodiments, the detecting comprises detecting MYC and/or MKLP2 RNA. In certain embodiments, the detecting comprises performing RNA sequencing, a Taqman assay, or fluorescence in situ hybridization (FISH) on the biological sample or cancer cells. In certain embodiments, the detecting comprises detecting an abnormality in the MYC gene and/or the MKLP2 gene. In certain embodiments, the detecting the abnormality in the MYC gene, comprises detecting a mutation in MYC, a translocation of MYC, a copy number of MYC, or combinations thereof. In certain embodiments, the detecting the abnormality in MKLP2 gene comprises detecting a mutation in MKLP2, a copy number of MKLP2, a copy number of MKLP2, or combinations thereof. In certain embodiments, the detecting MYC expression and/or activity comprises detecting MYCN and/or MYCL. In certain embodiments, the detecting comprises detecting a transcriptional profile of a cancer-dependent gene signature and/or detecting a MYC-dependent cellular phenotypic signature. In certain embodiments, detecting the transcriptional profile of a cancer-dependent gene signature comprises detecting expression of MYC and MKLP2, wherein detecting expression of MYC and MKLP2 comprises sequencing RNA derived from the biological sample or cancer cells. In certain embodiments, detecting the MYC-dependent cellular phenotypic signature comprises performing an image-based screening assay, a 3-(4,5-dimethylthiazol-2-yl)-2,5-diphenyltetrazolium bromide tetrazolium (MTT) assay, a CellTiter-Glo® (CTG) assay, a lactate dehydrogenase (LDH) assay, or combinations thereof. In certain embodiments, the image-based screening assay comprises detection of mitotic arrest, detection of induction of polyploidy, detection of cell death, an immunofluorescent assay, or combinations thereof.

In certain aspects, described herein are methods of detecting expression and/or activity of MYC and MKLP2, comprising: contacting a biological sample obtained from an individual diagnosed with cancer or experiencing one or more symptoms associated with cancer with at least a first and second test agent; wherein the first test agent detects MYC expression and/or activity and the second test agent detects MKLP2 expression and/or activity. In certain embodiments, the method further comprises detecting expression of MYC and/or MKLP2. In certain embodiments, the first test agent comprises an antibody that binds MYC. In certain embodiments, the second test agent comprises an antibody that binds MKLP2. In certain embodiments, expression of MYC and/or MKLP2 is quantified. In certain embodiments, the quantification comprises quantification of an image obtained from an immunohistochemistry assay, a FISH assay, an RNA-seq, a Taqman, quantitative PCR, proteomics assay, an immunoblot, or an ELISA. In certain embodiments, the individual is identified as an individual who may benefit from a treatment comprising one or more agent(s) that inhibit cancer cell viability and/or proliferation if expression of MYC is elevated relative to a suitable control and expression of MKLP2 is decreased relative to a suitable control. In certain embodiments, the suitable control comprises expression of β-Actin in the same biological sample.

In certain embodiments of the methods described herein, the biological sample comprises lung cells, breast cells, blood cells, brain cells, ovary cells, skin cells, intestine cells, pancreas cells, bone cells, kidney cells, prostate cells, stomach cells, cervix cells, or liver cells. In certain embodiments, the biological sample comprises biopsied tissue. In certain embodiments, the biopsied tissue comprises biopsied tumor tissue.

In certain embodiments, of the methods described herein, MYC comprises the MYC protein family, wherein the MYC protein family comprises one or more of MYC, MYCN and MYCL.

In certain embodiments of the methods described herein, the cancer is bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, soft tissue sarcoma, melanoma, astrocytoma, oligoastrocytoma, bone cancer, brain cancer, gastrointestinal cancer, cardiac cancer, uterine cancer, head and neck cancer, gallbladder cancer, laryngeal cancer, lip and oral cavity cancer, ocular cancer, colorectal cancer, testicular cancer, throat cancer, acute lymphoblastic leukemia (ALL), chronic myelogenous leukemia (CML), adrenocortical carcinoma, AIDS-related lymphoma, primary CNS lymphoma, anal cancer, appendix cancer, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, extrahepatic ductal carcinoma in situ (DCIS), endometrial cancer, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, myelodysplastic syndromes, nasal cavity and paranasal sinus cancer, nasopharyngeal cancer, epithelial ovarian cancer, germ cell ovarian cancer, low malignant potential ovarian cancer, papillomatosis, paraganglioma, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, sézary syndrome, small intestine cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or waldenström macroglobulinemia.

In certain embodiments of the methods described herein, the suitable control is a biological sample without cancer. In certain embodiments, the suitable control is a biological sample without cancer from the individual. In certain embodiments, the suitable control is a predetermined threshold determined from a biological sample obtained from individuals or tissues without cancer.

In certain embodiments, the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC. In certain embodiments, the one or more agent(s) that inhibit cancer cell viability and/or proliferation is selected from LC30, LW33R, LXY013, LXY018, LC02, LC09, LG157, LG160, LG169, LG171, LG172, LG177, and LG181.

In certain aspects, described herein are kits for detecting expression of MYC and MKLP2 in a biological sample obtained from an individual who may benefit from a treatment comprising one or more agent(s) that inhibit cancer cell viability and/or proliferation, the kit comprising: (i) at least a first and a second test agent, wherein the first test agent detects MYC expression and the second test agent detects MKLP2 expression; and (ii) instructions for use.

### BRIEF DESCRIPTION OF THE DRAWINGS

FIGS. 1A-1G are a schematic and a set of graphs, respectively, showing that depletion of the MYC gene with small interfering RNA (siRNA) knockdown makes cells less responsive to the compounds LC30 and LW33R. HeLa cells were transfected with siRNAs, treated with MYC synthetic lethal compounds, and analyzed using the protocol outlined in FIG. 1A. Concentration-response curves and images from light microscopy are shown for cells transfected with control versus a MYC siRNA combined with treatment with LC30 (FIG. 1B) or LW33R (FIG. 1C), respectively. A 24-well protocol that eliminated the splitting of cells, as outlined in the schematic depicted in FIG. 1D, was also tested. The degree of MYC protein was assessed in the 12-well assay after siRNA transfection (FIG. 1E). The percentage of viable and non-viable cells were quantified from a trypan blue exclusion assay from experiments in which cells were transfected with control or MYC siRNA and exposed to the compounds LC30 (FIG. 1F) or LW33R (FIG. 1G), respectively. For FIG. 1B, FIG. 1C, FIG. 1F, and FIG. 1G, statistical analysis were performed by two-way ANOVA, multiple comparisons, *p<0.05, **p<0.01, ***p<0.001, ****p<0.0001. All experiments were carried out in triplicate and data presented as an average±standard deviation.

FIGS. 2A-2F are a set of light microscopy images, graphs, and a table, respectively, showing that depletion of MYC with siRNA knockdown made cells less responsive to the compounds LC30 and LW33R. HeLa cells were transfected with siRNAs, treated with MYC synthetic lethal compounds, and analyzed using the protocol outlined (FIG. 2A). Concentration-response curves and images from light microscopy are shown for cells transfected with a control versus a MYC siRNA combined with treatment with LC30 (FIG. 2B) or LW33R (FIG. 2C). To determine if cell loss during the splitting of transfected cells influenced the observations in (FIG. 2B) and (FIG. 2C), a different, 24-well protocol was designed that circumvented the need to split cells, and the results are shown in FIG. 2D. The percent cell viability was assessed in the 12-well assay after siRNA transfection (FIG. 2E). The percentage of viable and non-viable cells were quantified from tryplan blue exclusion assays of cells transfected with control or MYC siRNA and then exposed to the compounds LC30 or LW33R (FIG. 2F). such versus For FIG. 2B, FIG. 2C, and FIG. 2F statistical analyses were performed by two-way ANOVA, multiple comparisons, *p<0.05, **p<0.01, ***p<0.001, ****p<0.0001. All experiments were carried out in triplicate and data presented as an average±standard deviation.

FIGS. 3A-3E are a set of graphs and photomicrographs, respectively, showing that compounds described herein elicit mitotic defects similar to those seen in cells treated with a known mitotic disruptor, paprotrain. NCI-H23 cells were treated with a MKLP2 inhibitor, paprotrain (FIG. 3A), or compound LG157 (FIG. 3B) for 6 hours to assay MKLP2 functionality during anaphase or at 72 hours to detect polyploidy. The proportion of cells with AURKB mis-localization was measured to assess the function of MKLP2 (FIG. 3C). Immunofluorescent analysis of localization of kinetochore proteins AURKB and CREST was determined. Scale bar=10 μm. FIG. 3D is an immunoblot depicting the results of an experiment in which NCI-H23 cells were transiently transfected with MKLP2 siRNA. MKLP2 knockdown efficiency was assayed by immunoblot blot analysis 72 hours after transfection. FIG. 3E are graphs depicting the proportion of cells with AURKB mis-localization after treatment with MKLP2 siRNA after 6 hours of exposure and the proportion of polyploid cells after 72 hours.

FIGS. 4A-4C are a set of graphs, photographs, and photomicrographs, respectively, showing inhibition of tumor growth in the Calu6 tumor model. Xenografted mice were treated with novel antimitotic compounds LXY013, LW33R, and LG157, respectively (FIG. 4A). p-values in FIG. 4A are from Logrank analysis. Tumor growth curves and body weight over the course of the experiments are depicted with vehicle control in black, test compounds in gray (n=6). FIG. 4B is a set of images of tumors and a graph depicting tumor weight at an end-point of 20 days. FIG. 4C is a set of photomicrographs of immunohistochemistry analyses and its quantification, respectively, of Ki-67 expression in xenografts. For LG157, * is p<0.05. p-values in FIG. 4B and FIG. 4C are from multi-parametric analysis. **** is p<0.0001.

FIGS. 5A-5F are a set of immunoblots and graphs, respectively, showing datasets of the drug response of human cell lines to MYC-synthetic lethal (SL) treatment. FIG. 5A is an immunoblot depicting MYC expression. FIG. 5B is an immunoblot depicting MKLP2 expression. The division of cell lines into MYC^{High} and MYC^{Low} (FIG. 5C) and MKLP2^{High} and MKLP2^{Low} (FIG. 5D) groups are quantified, respectively. In FIG. 5C and FIG. 5D, * indicates a p-value less than 0.05 with a Fisher's Exact Test. FIG. 5E is a graph depicting the concentration-response curves for LG157 in 98 different human cancer cell lines. FIG. 5F is a graph depicting concentration-response curve metrics for statistical analysis of the high vs. low protein responsivity groups.

FIGS. 6A-6F are a set of graphs showing that MYC and MKLP2 expression predict response to LG-157. FIG. 6A is a graph depicting the average concentration-response curves for the group of responsive cell lines (AUC<0.7730) and the less responsive (AUC≥0.7330). FIG. 6B is a graph of the responsive and less responsive groups, respectively, with each dot depicting is a cell line. FIG. 6C is a graph depicting the proportion of cell lines that were responsive to LG157, as compared to cells that were less responsive, as assessed through AUC measurement, of cell groups that fit into the MYC^{High} and MYC^{Low} category.

FIG. 6D is a graph depicting the proportion of cell lines that were responsive, as compared to cells that were less responsive, of cell groups that fit into the MKLP2^{High} and MKLP2^{Low} category. FIG. 6E is a graph depicting the proportion of cell lines that are responsive, as compared to cells that were less responsive, when analyzed with both MYC and MKLP2 protein expression, respectively. Statistics: p-values in FIGS. 6C-6E are from the Fisher's Exact Test. FIG. 6F is a graph depicting the correlation analysis between MYC and MKLP2 protein expression values.

FIGS. 7A-7F are a set of graphs showing that driver mutations common to cancer do not predict the response to LG-157, including the proportion of cell lines that were responsive versus those less responsive, as assessed through AUC measurement. FIG. 7A is a graph depicting the comparisons showing the proportion of cell lines that were responsive versus those less responsive between cell lines that harbored RAS activating mutation. FIG. 7B is a graph depicting a similar set of experiments but with comparisons shown between those that harbored one or both of a RAS/RAF mutation versus those that had wildtype RAS and RAF. while FIG. 7C shows tumors that had RB gene mutation versus those cell lines that had wildtype RB, and FIG. 7D shows cell lines that harbored a RAF-activating mutation versus those that had wildtype RAF. FIG. 7E is a similar set of experiments, though depicting cell lines that harbored PTEN mutation and/or an activating mutation in PI3K genes versus those that were wildtype for genes in the pathway, while FIG. 7F shows cell lines that harbored TP53 mutation versus those cell lines that had wildtype TP53. Statistics: p-values in FIGS. 7A-7F are from a Fisher's Exact Test.

FIGS. 8A-8G are a set of graphs which depict the comparison of drug sensitivity to LG157 (FIG. 8A), LC02 (FIG. 8B), LC09 (FIG. 8C), LXY018 (FIG. 8D), LXY013 (FIG. 8E), LC30 (FIG. 8F), and LW33R (FIG. 8G), respectively of the MYC^{High}/MKLP2^{Low} cells for multiple novel antimitotic compounds, as normalized to a reference group in which MYC^{High}/MKLP2^{High}, MYC^{Low}/MKLP2^{High}, and MYC^{Low}/MKLP2^{Low} groups were combined (“All Other”). Statistics: p-values in FIGS. 8A-8G are from a Fisher's Exact Test.

FIGS. 9A-9G are a set of graphs which depict the comparison of drug sensitivity to LG160 (FIG. 9A), LG169 (FIG. 9B), LG171 (FIG. 9C), LG172 (FIG. 9D), LG177 (FIG. 9E), LG181 (FIG. 9F), and SY99 (FIG. 9G), respectively of the MYC^{High}/MKLP2^{Low} cells for multiple novel antimitotic compounds, as normalized to a reference group in which MYC^{High}/MKLP2^{High}, MYC^{Low}/MKLP2^{High}, and MYC^{Low}/MKLP2^{Low} groups were combined (“All Other”). Statistics: p-values in FIGS. 9A-9G are from a Fisher's Exact Test.

FIG. 10 is a graph depicting the concentration-response curve for LC30 and LW33R respectively in the NCI-H520 cell line.

### DETAILED DESCRIPTION

### Definitions

The features and other details of the disclosure will now be more particularly described. Certain terms employed in the specification, examples, and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

As used herein, the singular form “a,” “an,” and “the” includes plural references unless indicated otherwise.

The term “about” means an acceptable error for a particular value as determined by one of ordinary skill in the art, which depends in part on how the value is measured or determined. In certain embodiments, the term “about” means within 5%, 4%, 3%, 2%, 1%, 0.5%, or 0.05% of a given value or range.

As used herein, “activity” refers to form(s) of a gene or respectively encoded protein which retains a biological activity of the native or naturally-occurring gene or polypeptide, respectively.

The term “administering,” or a grammatical derivative thereof, as described herein, refers to the delivery of an agent(s) that inhibit a cancer cell viability and/or proliferation response to an individual in need thereof. Any suitable method of administration can be selected by one of skill in the art, in view of this disclosure.

As used herein, an “agent that inhibits cancer cell viability and/or proliferation” is a molecule that decreases, blocks, inhibits, abrogates or interferes with the viability and/or proliferation of a cancer cell. In some embodiments, one or more agent(s) that inhibit cancer cell viability and/or proliferation include small molecule antagonists, polynucleotide antagonists, antibodies and antigen-binding fragments thereof, fusion proteins, oligopeptides, and other molecules that decrease, block, inhibit, abrogate or interfere with signal transduction resulting from the interaction of a one or more agent(s) that inhibit cancer cell viability and/or proliferation receptor with one or more of its binding partners.

The terms “benefits from treatment” as well as “treat,” “treatment,” “treating,” and the like are used herein to generally mean obtaining a desired pharmacological and/or physiological effect. The effect may be therapeutic in terms of partially or completely curing a disease and/or symptom(s) of the disease. The term “treatment” as used herein covers any treatment of cancer in a human, and includes: (a) inhibiting the cancer, i.e., preventing the cancer from increasing in severity or scope; (b) relieving the cancer, i.e., causing partial or complete amelioration of the cancer i.e., treating malignant progression; or (c) preventing relapse of the cancer, i.e., preventing the cancer from returning to an active state following previous successful treatment of symptoms of the disorder or treatment of the disorder i.e., treating malignant progression.

By “biological sample” or “sample” is meant a fluid or solid sample from an individual. Biological samples may include cells (e.g., lung cells, breast cells, blood cells, brain cells, ovary cells, skin cells, intestine cells, pancreas cells, bone cells, kidney cells, prostate cells, stomach cells, cervix cells, or liver cells) or blood or biological fluids including (e.g., plasma, blood, serum, stool, urine, or combinations thereof). Solid biological samples include samples obtained from a tissue biopsy, aspirate, or surgical removal or samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Fluid biological samples include samples taken from the blood, serum, plasma, urine, pancreatic fluid, CSF, semen, prostate fluid, seminal fluid, urine, saliva, sputum, mucus, bone marrow, lymph, and tears. In some embodiments, the biological sample is a tissue, plasma, blood, stool, urine, or combinations thereof. In some embodiments, the biological sample is obtained from a tissue biopsy, aspirate, or surgical removal. In some embodiments, the biological sample is lung cells, breast cells, blood cells, brain cells, ovary cells, skin cells, intestine cells, pancreas cells, bone cells, kidney cells, prostate cells, stomach cells, cervix cells, or liver cells. In some embodiments, the biological sample is biopsied tissue. In some embodiments, the biological sample is a sample derived from cancer cells.

As used herein, a “cancer-dependent gene signature” refers to a single or combined group of genes in a cell with a characteristic pattern of gene expression that occurs as a result of cancer. For example, as used herein, genes of a “cancer-dependent gene signature” can refer to one or more of MYC (e.g., MYC, MYCN and MYCL) and MKLP2.

The terms “control,” “reference,” and “suitable control” are meant to mean any useful reference, for example, to compare the expression and/or activity of the one or more genes of the cancer-dependent gene signature or to compare a phenotype (e.g., morphology). The baseline can be any sample, standard, standard curve, or level that is used for comparison purposes. The baseline can be a normal reference sample or a reference standard or level. A “suitable control” can be, for example, a control, e.g., a predetermined negative control value such as a “normal control” or a prior sample taken from the same individual; a sample from a normal healthy individual, a sample from an individual not having cancer; or a sample from an individual that has been treated for cancer. By “reference standard or level” is meant a value or number derived from a reference sample. A “normal control value” is a pre-determined value indicative of non-disease state, e.g., a value expected in a healthy control individual. A normal control value can be expressed as a range (“between X and Y”), a high threshold (“no higher than X”), or a low threshold (“no lower than X”). An individual having a measured value within the normal control value for a particular assay can be referred to as “within normal limits” for that assay. A normal reference standard or level can be a value or number derived from a normal individual not having cancer; or an individual that has been treated for cancer. In some embodiments, the reference sample, standard, or level is matched to the individual sample by at least one of the following criteria: age, weight, sex, disease stage, and overall health. As used herein, a “suitable control” can refer to the expression and/or activity levels of one or more (e.g., two, three, or four) genes of the cancer-dependent gene signature against which the expression and/or activity levels of the respective genes are compared, e.g., to make a diagnostic, predictive, prognostic, and/or therapeutic determination. As used herein, a “suitable control” can refer to a phenotypic marker of the MYC-dependent cellular phenotypic signature against which the comparison of the respective phenotype is compared, e.g., to make a diagnostic, predictive, prognostic, and/or therapeutic determination. In some embodiments, a suitable control includes substantially no test agent administered to an individual. In some embodiments, a suitable control is a biological sample without cancer (e.g., a biological sample or tissues obtained from an individual without cancer). In some embodiments, a suitable control is a predetermined threshold determined from a biological sample obtained from individuals or tissues without cancer. In some embodiments, a suitable control is the expression of β-Actin or other housekeeping genes, such as, for example, glyceraldehyde-3-phosphate dehydrogenase, phosphoglycerate kinase 1, beta-2-microglobulin, and TATA box binding protein, etc. in a biological sample. As used herein, a “housekeeping gene,” refers to a constitutive gene that is required for the maintenance of basal cellular functions that are essential for the existence of a cell, regardless of its specific role in the tissue or organism. Thus, housekeeping genes are expressed in most or all cells of an organism under normal and patho-physiological conditions, irrespective of tissue type, developmental stage, cell cycle state, or external signal.

Throughout the specification and claims, the word “comprise,” or variations such as “comprises” or “comprising,” will be understood to imply the inclusion of a stated word or group of words but not the exclusion of any other word or group of words.

The term “detection” includes any means of detecting known in the art, including direct and indirect detection.

By “detecting RNA” is meant the detection of a nucleic acid (e.g., mRNA) by methods known in the art. Methods to measure mRNA levels generally include, but are not limited to, northern blotting, nuclease protection assays (NPA), in situ hybridization (ISH), reverse transcription-polymerase chain reaction (RT-PCR), and RNA sequencing (RNA-Seq).

By “detecting protein” is meant the detection of a protein by methods known in the art. Methods to measure protein levels generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, florescent polarization, phosphorescence, immunohistochemical analyses, matrix-associated laser desorption/ionization time of light (MALDI-TOF) mass spectrometry, liquid chromatography (LC)-mass spectrometry, microcytometry, microscopy, florescence activated cell coating (FACs), and flow cytometry, as well as assays based on a property of a protein including, but not limited to, enzymatic activity or interaction with other proteins (e.g., substrates or other proteins of a protein complex).

The terms “diagnose, “diagnosing,” “diagnosis,” and “diagnosed” are used herein to refer to the identification or classification of a genetic, molecular, or pathological state, disease, or condition (e.g., cancer). For example, “diagnosed” may refer to identification of an individual with cancer.

As used herein, the terms “effective amount,” “therapeutically effective amount,” and the like, when used in reference to a method described herein, refer to a quantity sufficient to, when administered to an individual, including human, effect beneficial or desired results (e.g., alleviate one or more symptoms of cancer), which may include clinical results. For example, an effective amount of one or more (e.g., two, three, or four) agents described herein (e.g., agent(s) that inhibit a cancer cell viability and/or proliferation response) may alleviate one or more symptoms of cancer as compared to the alleviation of said symptom without administration of the agent of interest. An “effective amount,” “therapeutically effective amount,” and the like, of an agent, such as one or more agent(s) that inhibit cancer cell viability and/or proliferation, also include an amount that results in a beneficial or desired result in an individual as compared to a control.

The phrase “identifying an individual” or “identifies an individual,” as used herein, refers to using the information or data generated by the methods described herein (e.g., information or data related to the expression and/or activity of the one or more, e.g., two, three, or four genes of the cancer-dependent gene signature) to identify or select an individual as likely to benefit or less likely to benefit from a therapy including one or more agents that inhibit cancer cell viability and/or proliferation. The information or data used or generated may by be in any form, written, oral, or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit, or combination thereof. In some embodiments, the information or data includes a comparison of the expression and/or activity of the one or more (e.g., two, three, or four) genes of the cancer-dependent gene signature or the MYC-dependent cellular phenotypic signature to a reference level. In some embodiments, the information or data includes an indication that the expression and/or activity of the one or more (e.g., two, three, or four) genes of the cancer-dependent gene signature are elevated or decreased relative to a suitable control (e.g., a control with substantially no test agent). In some embodiments, the information or data includes an indication that the individual has or does not have an elevated risk for cancer.

As used herein, the terms “individual,” “subject,” and “patient” are used interchangeably and are meant as a human. An individual to be treated with a pharmaceutical composition (e.g., a pharmaceutical composition including one or more agent(s) that inhibit cancer cell viability and/or proliferation) described herein may be one who has been diagnosed by a medical practitioner as having cancer or one at risk for developing cancer.

By “level” is meant a level of a genes expression or activity as compared to a reference. The reference can be any useful reference, as defined herein. By a “decreased level” or an “increased level” of a gene is meant a decrease or increase in gene expression or activity, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). The terms “level of expression” or “expression level” in general are used interchangeably and generally refer to the amount of a biomarker (e.g., one or more, two, three, or four) genes of the cancer-dependent gene signature in a biological sample). “Expression” generally refers to the process by which information (e.g., gene-encoded and/or epigenetic information) is converted into the structures present and operating in a cell. Therefore, as used herein, “expression” may refer to transcription into a polynucleotide translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modifications of a polypeptide) shall also be regarded as expressed whether they originate from a transcript generated by alternative splicing or a degraded transcript, or from post-translational processing of a polypeptide, e.g. by proteolysis. As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

The term “modified” as used herein, refers to an observable difference in the level of a marker, such as the expression and/or activity of one or more (e.g., two, three, or four) gene(s), in a sample (e.g., a biological sample from an individual e.g., an individual suspected of being at risk of developing cancer or diagnosed with cancer), as determined using techniques and methods known in the art for the measurement of the marker. A marker level that is changed in an individual may result in a difference of at least 1% (e.g., at least 5%, 10%, 25%, 50%, or 100% or at least 2.5-fold, 3-fold, 4-fold, 5-fold, 6-fold, 7-fold or more) than a reference level. In some embodiments, the change is an increase in the level of the expression or activity of the one or more (e.g., two, three, or four) genes of the cancer-dependent gene signature or a modification in the MYC-dependent cellular phenotypic signature in a biological sample from an individual.

As used herein, a “MYC-dependent cellular phenotypic signature” refers to a single or combined group of phenotypes in a cell that occurs as a result of MYC expression. For example, as used herein, phenotypes of a “MYC-dependent cellular phenotypic signature” can refer to one or more of phenotypes related to cell viability and suppression of cytokinesis.

As used herein, abbreviations in the application include, but are not limited to, aurora kinase A (AURKA), aurora kinase B (AURKB), aurora kinase C (AURKC), B-cell lymphoma 2 (BCL-2), B-cell lymphoma extra-large (BCL-XL), checkpoint kinase 1 (Chk1), cyclin-dependent kinase 1 (CDK1), cyclin-dependent kinase 2 (CDK2), cyclin-dependent kinase 4 (CDK4), cyclin-dependent kinase 5 (CDK5), cyclin-dependent kinase 6 (CDK6), cyclin-dependent kinase 9 (CDK9), centromere protein E (CENP-E), kinesin-5, putative (EG5), histone deacetylase (HDAC), kidney-specific cadherin (KSP-cadherin), targeting kinase-like protein 18A (KIF18A), mitotic kinesin-like protein 1 (MKLP1), mitotic kinesin-like protein 2 (MKLP2), mammalian target of rapamycin (mTOR), Rho-associated, coiled-coil-containing protein kinase (ROCK), Rho-associated, coiled-coil-containing protein kinase 1 (ROCK1), Rho-associated, coiled-coil-containing protein kinase 2 (ROCK2), phosphoinositide 3-kinase (PI3K), phosphoinositide 3-kinase a (PI3Kα), phosphoinositide 3-kinase y (PI3Ky), phosphoinositide 3-kinase 8 (PI3K8), polo like kinase 1 (PLK1), polo like kinase 2 (PLK2), polo like kinase 3 (PLK3), and polo like kinase 4 (PLK4).

### Cancer

The methods described herein can be used to diagnose, prognose, and treat cancer as well as screening compounds for the treatment of cancer. In some embodiments, the cancer is bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, soft tissue sarcoma, melanoma, astrocytoma, oligoastrocytoma, bone cancer, brain cancer, gastrointestinal cancer, cardiac cancer, uterine cancer, head and neck cancer, gallbladder cancer, laryngeal cancer, lip and oral cavity cancer, ocular cancer, colorectal cancer, testicular cancer, throat cancer, acute lymphoblastic leukemia (ALL), chronic myelogenous leukemia (CML), adrenocortical carcinoma, AIDS-related lymphoma, primary CNS lymphoma, anal cancer, appendix cancer, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, extrahepatic ductal carcinoma in situ (DCIS), endometrial cancer, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, myelodysplastic syndromes, nasal cavity and paranasal sinus cancer, nasopharyngeal cancer, epithelial ovarian cancer, germ cell ovarian cancer, low malignant potential ovarian cancer, papillomatosis, paraganglioma, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, sézary syndrome, small intestine cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or waldenström macroglobulinemia.

For example, in some embodiments, the cancer is bladder cancer. In some embodiments, the cancer is pancreatic cancer. In some embodiments, the cancer is cervical cancer. In some embodiments, the cancer is lung cancer. In some embodiments, the cancer is liver cancer. In some embodiments, the cancer is ovarian cancer. In some embodiments, the cancer is colon cancer. In some embodiments, the cancer is stomach cancer. In some embodiments, the cancer is virally induced cancer. In some embodiments, the cancer is neuroblastoma. In some embodiments, the cancer is breast cancer. In some embodiments, the cancer is prostate cancer. In some embodiments, the cancer is renal cancer. In some embodiments, the cancer is leukemia. In some embodiments, the cancer is sarcoma. In some embodiments, the cancer is carcinoma. In some embodiments, the cancer is non-small cell lung carcinoma. In some embodiments, the cancer is non-Hodgkin's lymphoma. In some embodiments, the cancer is AML. In some embodiments, the cancer is CLL. In some embodiments, the cancer is B-CLL. In some embodiments, the cancer is MM. In some embodiments, the cancer is erythroleukemia. In some embodiments, the cancer is renal cell carcinoma. In some embodiments, the cancer is soft tissue sarcoma. In some embodiments, the cancer is melanoma. In some embodiments, the cancer is astrocytoma. In some embodiments, the cancer is oligoastrocytoma. In some embodiments, the cancer is bone cancer. In some embodiments, the cancer is brain cancer. In some embodiments, the cancer is gastrointestinal cancer. In some embodiments, the cancer is cardiac cancer. In some embodiments, the cancer is uterine cancer. In some embodiments, the cancer is head and neck cancer. In some embodiments, the cancer is gallbladder cancer. In some embodiments, the cancer is laryngeal cancer. In some embodiments, the cancer is lip and oral cavity cancer. In some embodiments, the cancer is ocular cancer. In some embodiments, the cancer is colorectal cancer. In some embodiments, the cancer is testicular cancer. In some embodiments, the cancer is throat cancer. In some embodiments, the cancer is ALL. In some embodiments, the cancer is CML. In some embodiments, the cancer is adrenocortical carcinoma. In some embodiments, the cancer is AIDS-related lymphoma. In some embodiments, the cancer is primary CNS lymphoma. In some embodiments, the cancer is anal cancer. In some embodiments, the cancer is appendix cancer. In some embodiments, the cancer is atypical teratoid/rhabdoid tumor. In some embodiments, the cancer is basal cell carcinoma. In some embodiments, the cancer is bile duct cancer. In some embodiments, the cancer is extrahepatic cancer. In some embodiments, the cancer is ewing sarcoma family. In some embodiments, the cancer is osteosarcoma and malignant fibrous histiocytoma. In some embodiments, the cancer is central nervous system embryonal tumors. In some embodiments, the cancer is central nervous system germ cell tumors. In some embodiments, the cancer is craniopharyngioma. In some embodiments, the cancer is ependymoma. In some embodiments, the cancer is bronchial tumors. In some embodiments, the cancer is burkitt lymphoma. In some embodiments, the cancer is carcinoid tumor. In some embodiments, the cancer is primary lymphoma. In some embodiments, the cancer is chordoma. In some embodiments, the cancer is chronic myeloproliferative neoplasms. In some embodiments, the cancer is extrahepatic DCIS. In some embodiments, the cancer is endometrial cancer. In some embodiments, the cancer is esophageal cancer. In some embodiments, the cancer is esthesioneuroblastoma. In some embodiments, the cancer is extracranial germ cell tumor. In some embodiments, the cancer is extragonadal germ cell tumor. In some embodiments, the cancer is fallopian tube cancer. In some embodiments, the cancer is fibrous histiocytoma of bone. In some embodiments, the cancer is gastrointestinal carcinoid tumor. In some embodiments, the cancer is GIST. In some embodiments, the cancer is testicular germ cell tumor. In some embodiments, the cancer is gestational trophoblastic disease. In some embodiments, the cancer is glioma. In some embodiments, the cancer is childhood brain stem glioma. In some embodiments, the cancer is hairy cell leukemia. In some embodiments, the cancer is hepatocellular cancer. In some embodiments, the cancer is langerhans cell histiocytosis. In some embodiments, the cancer is hodgkin lymphoma. In some embodiments, the cancer is hypopharyngeal cancer. In some embodiments, the cancer is islet cell tumors. In some embodiments, the cancer is pancreatic neuroendocrine tumors. In some embodiments, the cancer is wilms tumor and other childhood kidney tumors. In some embodiments, the cancer is langerhans cell histiocytosis. In some embodiments, the cancer is small cell lung cancer. In some embodiments, the cancer is cutaneous T-cell lymphoma. In some embodiments, the cancer is intraocular melanoma. In some embodiments, the cancer is merkel cell carcinoma. In some embodiments, the cancer is mesothelioma. In some embodiments, the cancer is metastatic squamous neck cancer. In some embodiments, the cancer is midline tract carcinoma. In some embodiments, the cancer is multiple endocrine neoplasia syndromes. In some embodiments, the cancer is myelodysplastic syndromes. In some embodiments, the cancer is nasal cavity and paranasal sinus cancer. In some embodiments, the cancer is nasopharyngeal cancer. In some embodiments, the cancer is epithelial ovarian cancer. In some embodiments, the cancer is germ cell ovarian cancer. In some embodiments, the cancer is low malignant potential ovarian cancer. In some embodiments, the cancer is papillomatosis. In some embodiments, the cancer is paraganglioma. In some embodiments, the cancer is parathyroid cancer. In some embodiments, the cancer is penile cancer. In some embodiments, the cancer is pharyngeal cancer. In some embodiments, the cancer is pheochromocytoma. In some embodiments, the cancer is pituitary tumor. In some embodiments, the cancer is pleuropulmonary blastoma. In some embodiments, the cancer is primary peritoneal cancer. In some embodiments, the cancer is rectal cancer. In some embodiments, the cancer is retinoblastoma. In some embodiments, the cancer is rhabdomyosarcoma. In some embodiments, the cancer is salivary gland cancer. In some embodiments, the cancer is kaposi sarcoma. In some embodiments, the cancer is sezary syndrome. In some embodiments, the cancer is small intestine cancer. In some embodiments, the cancer is thymoma and thymic carcinoma. In some embodiments, the cancer is thyroid cancer. In some embodiments, the cancer is transitional cell cancer of the renal pelvis and ureter. In some embodiments, the cancer is urethral cancer. In some embodiments, the cancer is endometrial uterine cancer. In some embodiments, the cancer is uterine sarcoma. In some embodiments, the cancer is vaginal cancer. In some embodiments, the cancer is vulvar cancer. In some embodiments, the cancer is waldenström macroglobulinemia.

### Genes of the Cancer-Dependent Signature

Applicants have discovered that the mRNA expression levels and/or activity of certain genes can be utilized to diagnose, prognose, and treat cancer, as well as to select individuals who would benefit from a treatment that includes one or more agent(s) that inhibit cancer cell viability and/or proliferation response. The expression and/or activity levels of such genes can also be used for screening compounds that reduce the risk of an individual developing cancer, reduce the risk of an individual developing one or more symptoms of cancer, and/or alleviate one or more symptoms of cancer. Exemplary, non-limiting genes, whose expression and/or activity which are of interest in the methods of the invention, include MYC (e.g., MYC, MYCN and MYCL) and MKLP2.

In some embodiments, MYC includes the MYC protein family, which includes MYC, MYCN and MYCL. In some embodiments, MYC is one or more of MYC, MYCN and MYCL. In some embodiments, MYC is MYC. In some embodiments, MYC is MYCN. In some embodiments, MYC is MYCL.

In some embodiments, a gene of the cancer-dependent gene signature is one or more (e.g., two, three, or four) genes selected from the list including, but not limited to: MYC, MYCN and MYCL, and MKLP2.

For example, in some embodiments, a gene of the cancer-dependent gene signature is MYC. In some embodiments, a gene of the cancer-dependent gene signature is MYCN. In some embodiments, a gene of the cancer-dependent gene signature is MYCL. In some embodiments, a gene of the cancer-dependent gene signature is MKLP2.

Such a cancer-dependent gene signature can be determined, for example, by a method including obtaining a dataset comprising data associated with expression and/or activity of MYC and MKLP2 in a biological sample obtained from the individual; and identifying that the cancer-dependent gene signature is modified when the expression and/or activity of MYC is increased and the expression and/or activity of MKLP2 is decreased relative to a suitable control.

For example, in some embodiments, the method includes obtaining a dataset comprising data associated with expression and/or activity of MYC and MKLP2 in a biological sample obtained from the individual; and identifying that the cancer-dependent gene signature is modified when the expression and/or activity of MYC is increased relative to a suitable control.

In some embodiments, the method includes obtaining a dataset comprising data associated with expression and/or activity of MYC and MKLP2 in a biological sample obtained from the individual; and identifying that the cancer-dependent gene signature is modified when the expression and/or activity of MKLP2 is decreased relative to a suitable control.

In some embodiments, the method includes contacting cancer cells or a sample derived from cancer cells with one or more test agents; detecting the expression and/or activity of MYC and MKLP2 in the cancer cells; and if the test agent increases the expression and/or activity of MYC and/or reduces the expression and/or activity of MKLP2 relative to a suitable control, identifying that the cancer-dependent gene signature has been modified by said test agent.

### Methods of the Disclosure

The present invention relates to the identification of the MYC-dependent cellular phenotypic signature as well as biomarker (e.g., one or more (e.g., two, three, or four) genes of the cancer-dependent gene signature that identify individuals at risk of developing cancer, suffering from one or more symptoms associated with cancer, or diagnosed with cancer. The differential expression and/or activity levels of genes of cancer-dependent gene signature) can be used to diagnose, prognose, and classify individuals with cancer from suitable controls (e.g., healthy controls). Alternatively, or in addition to the differential expression and/or activity level of genes, the MYC-dependent cellular phenotypic signature can be used to diagnose, prognose, and classify individuals with cancer from suitable controls (e.g., healthy controls). Accordingly, the methods described herein are useful for treating or diagnosing cancer.

These methods may be carried out generally as described above or as known in the art with respect to sample collection and assay format.

### Methods of Treatment

The invention also features a method of treating an individual at risk of developing cancer, suffering from one or more symptoms associated with cancer, and/or diagnosed with cancer including administering to the individual suffering from one or more symptoms associated with cancer and/or diagnosed with cancer a therapeutically effective amount of a one or more agent(s) that inhibit cancer cell viability and/or proliferation.

Also provided herein, in some embodiments are methods of treating an individual at risk of developing cancer, suffering from one or more symptoms associated with cancer, and/or diagnosed with cancer, including: obtaining a dataset including data associated with expression and/or activity of MYC and MKLP2 in a biological sample obtained from the individual; and identifying the individual as at risk of developing cancer or having cancer when the expression and/or activity of MYC is increased and the expression and/or activity of MKLP2 is decreased relative to a suitable control. In some embodiments, the method further includes administering to the individual one or more agent(s) that inhibit cancer cell viability and/or proliferation (e.g., LC30, LXY013, LW33R, LG157.

Also provided herein, in some embodiments, are methods of treating a cancer with increased expression and/or activity of MYC and decreased expression and/or activity of MKLP2 in an individual, the method including administering to the individual one more agent(s) that inhibit cancer cell viability and/or proliferation. In some embodiments, the one or more agent(s) that inhibit cancer cell viability and/or proliferation comprises an agent that dysregulates MYC, one or more mitotic kinase(s), one or more mitotic motor protein(s), and/or an upstream regulator or a downstream effector of the CPPC. In some embodiments, the one or more agent(s) that inhibit cancer cell viability and/or proliferation is LC30, LW33R, LXY013, LXY018, LC02, LC09, LG157, LG160, LG169, LG171, LG172, LG177, and LG181. For example, in some embodiments, the agent that inhibits cancer viability and/or proliferation is LC30. In some embodiments, the agent that inhibits cancer viability and/or proliferation is LW33R, In some embodiments, the agent that inhibits cancer viability and/or proliferation is LXY013. In some embodiments, the agent that inhibits cancer viability and/or proliferation is LXY018. In some embodiments, the agent that inhibits cancer viability and/or proliferation is LC02. In some embodiments, the agent that inhibits cancer viability and/or proliferation is LC09. In some embodiments, the agent that inhibits cancer viability and/or proliferation is LG157. In some embodiments, the agent that inhibits cancer viability and/or proliferation is LG160. In some embodiments, the agent that inhibits cancer viability and/or proliferation is LG169. In some embodiments, the agent that inhibits cancer viability and/or proliferation is LG171. In some embodiments, the agent that inhibits cancer viability and/or proliferation is LG172. In some embodiments, the agent that inhibits cancer viability and/or proliferation is LG177. In some embodiments, the agent that inhibits cancer viability and/or proliferation is LG181.

In some embodiments, the method further includes obtaining the biological sample from the individual. In some embodiments, the method further includes processing the sample to produce the dataset.

The invention also features a method for treatment of cancer in an individual by obtaining a biological sample (e.g., tissue, plasma, blood, stool, or urine) from the individual suspected of being at risk of developing cancer, suffering from one or more symptoms associated with cancer, or diagnosed with cancer; detecting the expression and/or activity of one or more (e.g., two, three, or four) genes of the cancer-dependent gene signature (e.g., MYC and/or MKLP2); identifying an individual at risk of developing cancer or diagnosed with cancer when the expression and/or activity of the one or more (e.g., two, three, or four) genes of the cancer-dependent gene signature are modified relative to a suitable control (e.g., a control with substantially no test agent); and administering to the individual identified as at risk of developing cancer or diagnosed with cancer one or more (e.g., two, three, or four) agent(s) that inhibit cancer cell viability and/or proliferation.

In some embodiments, the method includes processing a cell obtained from the biological sample to produce a test cell.

In some embodiments, the method further includes contacting the biological sample or cells with a one or more agent(s) that inhibit cancer cell viability and/or proliferation (e.g., LC30, LW33R, LXY013, LXY018, LC02, LC09, LG157, LG160, LG169, LG171, LG172, LG177, and LG181) prior to detecting the expression level and/or activity of one or more genes of the cancer-dependent gene signature.

For example, in some embodiments, the method includes treating an individual at risk of developing cancer, suffering from one or more symptoms associated with cancer, and/or diagnosed with cancer by obtaining a biological sample from the individual suspected of being at risk of developing cancer, suffering from one or more symptoms associated with cancer, or diagnosed with cancer; processing a cell obtained from the biological sample to produce a test cell; contacting the test cell with a one or more agent(s) that inhibit cancer cell viability and/or proliferation (e.g., LC30, LW33R, LXY013, LXY018, LC02, LC09, LG157, LG160, LG169, LG171, LG172, LG177, and LG181) to produce cancer cell viability and/or proliferation-related response; detecting the expression and/or activity of one or more genes of the cancer-dependent gene signature; identifying the individual as at risk of developing cancer or diagnosing the individual with cancer when the expression and/or activity of the one or more genes are modified relative to a suitable control (e.g., a control with substantially no test agent); and administering to the individual identified as at risk of developing cancer or diagnosed with cancer one or more agents that inhibit cancer cell viability and/or proliferation-induced response.

The methods can also be used to determine the proper dosage (e.g., the therapeutically effective amount) of a therapeutic agent for the individual, the proper type of therapeutic agent, or whether a therapy should be administered.

In some embodiments, the method includes treating an individual at risk for developing cancer, diagnosed with cancer, or experiencing one or more symptoms associated with cancer by administering one or more agent(s) that inhibit cancer cell viability and/or proliferation-induced response.

The methods of the disclosure also include prophylactic treatments. For example, the disclosure also provides a method of preventing cancer in an individual at risk of developing cancer including an effective amount of a one or more agent(s) that inhibit cancer cell viability and/or proliferation.

### Diagnostic and Classification Methods

The present invention features methods to diagnose cancer. For example, in some embodiments, provided herein are methods for identifying an individual who may benefit from a treatment comprising one or more agent(s) that inhibit cancer cell viability and/or proliferation (e.g., LC30, LW33R, LXY013, LXY018, LC02, LC09, LG157, LG160, LG169, LG171, LG172, LG177, and LG181), including: obtaining a dataset associated with expression and/or activity of MYC in a biological sample obtained from the individual; wherein an expression and/or activity of MYC that is elevated relative to a suitable control identifies the individual as one who may benefit from a treatment including one or more agent(s) that inhibit cancer cell viability and/or proliferation; and wherein the one or more agent(s) that inhibit cancer cell viability and/or proliferation includes an agent that dysregulates MYC, one or more mitotic kinase(s), one or more mitotic motor protein(s), and/or an upstream regulator or a downstream effector of the chromosomal passenger protein complex (CPPC).

Also provided herein, in some embodiments, are methods for identifying an individual who may benefit from a treatment comprising one or more agent(s) that inhibit cancer cell viability and/or proliferation (e.g., LC30, LW33R, LXY013, LXY018, LC02, LC09, LG157, LG160, LG169, LG171, LG172, LG177, and LG181), including: obtaining a dataset including data associated with expression and/or activity of MKLP2 in biological sample obtained from the individual; wherein an expression and/or activity of MKLP2 that is decreased relative to a suitable control identifies the individual as one who may benefit from a treatment including one or more agent(s) that inhibit cell viability; and wherein the one or more agent(s) that inhibit cancer cell viability and/or proliferation includes an agent that dysregulates MYC, one or more mitotic kinase(s), one or more mitotic motor protein(s), and/or an upstream regulator or a downstream effector of CPPC.

Also provided herein, in some embodiments, are methods for identifying an individual who may benefit from a treatment including one or more agent(s) that inhibit cancer cell viability and/or proliferation (e.g., LC30, LW33R, LXY013, LXY018, LC02, LC09, LG157, LG160, LG169, LG171, LG172, LG177, and LG181), including: obtaining a dataset including data associated with expression and/or activity of MYC and MKLP2 in a biological sample obtained from the individual; wherein an expression and/or activity of MYC that is elevated relative to a suitable control and an expression and/or activity of MKLP2 that is decreased relative to a suitable control identifies an individual as one who may benefit from a treatment including one or more agent(s) that inhibit cancer cell viability and/or proliferation.

In some embodiments, the method further includes obtaining the biological sample from the individual. In some embodiments, the method further includes processing the sample to produce the dataset.

The methods of the invention may be used alone or as a companion diagnostic with other diagnostic or therapeutic approaches, as an early molecular screen to distinguish cancer. More specifically, alterations in the expression level and/or activity of one or more (e.g., two, three, or four) genes of the cancer-dependent gene signature, exemplified herein (e.g., MYC and/or MKLP2) in a biological sample (e.g., tissue, plasma, blood, stool, or urine) from the individual suspected of being at risk of developing cancer, suffering from one or more symptoms associated with cancer, or diagnosed with cancer as compared to a suitable control (e.g., a normal reference such as a control with substantially no test agent) can be used to diagnose cancer from diseases or disorders with similar symptoms, thereby allowing individual classification.

In some embodiments, the method further includes contacting the biological sample or cells with a one or more agent(s) that inhibit cancer cell viability and/or proliferation (e.g., LC30, LW33R, LXY013, LXY018, LC02, LC09, LG157, LG160, LG169, LG171, LG172, LG177, and LG181) prior to detecting the expression level and/or activity of one or more genes of the cancer-dependent gene signature.

For example, in some embodiments, the method includes identifying an individual at risk of developing cancer or diagnosed with cancer by obtaining a biological sample from the individual suspected of being at risk of developing cancer or diagnosed with cancer; processing a cell obtained from the biological sample to produce a test cell; contacting the test cell with a one or more agent(s) that inhibit cancer cell viability and/or proliferation (e.g., LC30, LW33R, LXY013, LXY018, LC02, LC09, LG157, LG160, LG169, LG171, LG172, LG177, and LG181) to produce a response; detecting whether induced response includes change in expression and/or activity of one or more (e.g., (e.g., two, three, or four) genes of the cancer-dependent gene signature e.g., MYC and/or MKLP2); and identifying the individual as at risk of developing cancer or diagnosing the individual with cancer if the expression and/or activity of the one or more genes are modified relative to a suitable control (e.g., a control with substantially no test agent).

The methods of the invention can be used to diagnose, prognose, or classify an individual, for example, an increase in the expression and/or activity (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of the biomarkers (e.g., MYC and/or MKLP2) may identify an individual as being at risk of developing cancer, suffering from one or more symptoms associated with cancer, diagnosed with cancer, and/or one who may benefit from one or more (e.g., two, three, or four) agent(s) that inhibit a cancer cell viability and/or proliferation response. Similarly, a decrease in the level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less, as compared to a reference) of the biomarkers (e.g., MYC and/or MKLP2) may identify an individual as being at risk of developing cancer, suffering from one or more symptoms associated with cancer, diagnosed with cancer, and/or one who may benefit from one or more (e.g., two, three, or four) agent(s) that inhibit a cancer cell viability and/or proliferation response.

Methods for Predicting and Monitoring Response to Agents that Inhibit the Cancer-Dependent Signature

The invention further features methods for predicting response to one or more (e.g., two, three, or four) agent(s) that inhibit cancer cell viability and/or proliferation in cells from individuals at risk of developing cancer, suffering from one or more symptoms associated with cancer, or diagnosed with cancer, before or after administration of one or more (e.g., two, three, or four) agent(s) that inhibit cancer cell viability and/or proliferation. For example, in some embodiments, the method includes screening compounds that reduce the risk of an individual developing cancer, reduce the risk of an individual developing one or more symptoms of cancer, and/or alleviate one or more symptoms of cancer in an individual by obtaining a biological sample from the individual at risk of developing cancer or suffering from cancer; processing a cell obtained from the biological sample to produce a test cell; contacting the test cell with a one or more agent(s) that inhibit cancer cell viability and/or proliferation to produce a cancer cell viability and/or proliferation-induced response; contacting the test cell with one or more test agents; detecting the expression and/or activity of one or more genes of the cancer-dependent gene signature; and if the one or more test agents modifies the expression and/or activity of one or more genes compared to a suitable control (e.g., a control with substantially no test agent), identifying the test agent as a compound that does reduce the risk of an individual developing cancer, reduce the risk of an individual developing one or more symptoms of cancer, and/or alleviate one or more symptoms of cancer in an individual.

For example, these methods may be carried out by obtaining cells from individuals at risk of developing cancer or suffering from cancer; contacting the cells with one or more (e.g., two, three, or four) test agents; detecting the expression and/or activity of one or more (e.g., two, three, or four) genes of the cancer-dependent gene signature (e.g., MYC and/or MKLP2) in the sample and/or determining if the test agent modifies the transcriptional profile of the cancer-dependent gene signature; and making a prediction about whether a test agent may reduce the risk of an individual developing cancer, reduce the risk of an individual developing one or more symptoms of cancer, and/or alleviate one or more symptoms of cancer in an individual. The method also can be used to predict whether an individual, who has been diagnosed with cancer, will respond positively to one or more (e.g., two, three, or four) agent(s) that inhibit cancer cell viability and/or proliferation.

In some embodiments, the method includes processing a cell obtained from the biological sample to produce a test cell.

In some embodiments, the method further includes contacting the biological sample or cells with a one or more agent(s) that inhibit cancer cell viability and/or proliferation (e.g., LC30, LW33R, LXY013, LXY018, LC02, LC09, LG157, LG160, LG169, LG171, LG172, LG177, and LG181) prior to detecting the expression level and/or activity of the one or more (e.g., two, three, or four) genes of the cancer-dependent gene signature.

A prediction of a positive response refers to a case where the cancer symptoms will be alleviated as a result of the one or more (e.g., two, three, or four) agent(s) that inhibit cancer cell viability and/or proliferation. For example, a positive response may include a reduction in malignant progression.

In the methods of predicting response to one or more (e.g., two, three, or four) agent(s) that inhibit cancer cell viability and/or proliferation, the transcriptional profile of the cancer-dependent gene signature can be determined relative to a control value. A control value can be a range or average value from a normal individual or a population of normal individuals; a value from a sample from an individual or population of individuals who have undergone treatment with one or more agent(s) that inhibit cancer cell viability and/or proliferation and have reduced symptoms following therapy; or a value from the same individual before the individual was diagnosed or before the individual started treatment.

The methods of the invention can be used to predict whether an individual will be responsive to one or more (e.g., two, three, or four) agent(s) that inhibit cancer cell viability and/or proliferation, for example, an increase in the level (e.g., an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more, as compared to a reference) of the expression and/or activity of biomarker(s) (e.g., MYC and/or MKLP2) may indicate a positive response to one or more (e.g., two, three, or four) agent(s) that inhibit cancer cell viability and/or proliferation. Similarly, a decrease in the level (e.g., a decrease by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; or a decrease by less than 0.01-fold, 0.02-fold, 0.1-fold, 0.3-fold, 0.5-fold, 0.8-fold, or less, as compared to a reference) of the expression and/or activity of biomarker(s) (e.g., MYC and/or MKLP2) may indicate a positive response to one or more (e.g., two, three, or four) agent(s) that inhibit cancer cell viability and/or proliferation.

The methods of the invention can be used to predict an individual's response to one or more (e.g., two, three, or four) agent(s) that inhibit cancer cell viability and/or proliferation and classify the individual as a “responder,” e.g., an individual with a cancer-dependent gene signature indicative of a positive response to one or more (e.g., two, three, or four) agent(s) that inhibit cancer cell viability and/or proliferation, or a “non-responder,” e.g., an individual with a cancer-dependent gene signature indicative of a poor response to one or more (e.g., two, three, or four) agent(s) that inhibit cancer cell viability and/or proliferation (e.g., an individual that may benefit from a different therapy other than, or in addition to, the respective one or more (e.g., two, three, or four) agent(s) that inhibit a cancer cell viability and/or proliferation response).

The prediction can be made prior to administration of a first agent that modifies the cancer cell viability and/or proliferation response. Alternatively, the prediction can be made after administration of the first agent that modifies the cancer cell viability and/or proliferation response, or after administration of a first agent that modifies the cancer cell viability and/or proliferation response but before a second agent that modifies the cancer cell viability and/or proliferation response. Furthermore, the prediction can be made at any time during the course of administration of one or more (e.g., two, three, or four) agent(s) that inhibit a cancer cell viability and/or proliferation response.

The methods described herein can also be used to monitor cancer status (e.g., progression or regression) during therapy or to optimize dosage of one or more (e.g., two, three, or four) therapeutic agents for an individual. For example, alterations (e.g., an increase or a decrease as compared to either the positive reference sample or the level diagnostic for cancer) can be detected to indicate an improvement in cancer status. In this embodiment, the levels of the cancer-dependent gene signature may be measured repeatedly as a method of not only diagnosing disorder, but also monitoring the treatment, prevention, or management of the disorder.

In order to monitor the status of cancer in an individual, individual samples may be compared to reference samples taken early in the diagnosis of the cancer. Such monitoring may be useful, for example, in assessing the efficacy of a particular therapeutic agent (e.g., one or more agent(s) that inhibit cancer cell viability and/or proliferation) in an individual, determining dosages, or in assessing disease progression or status. For example, the expression and/or activity of any of the genes described herein, or any combination thereof can be monitored in an individual, and as the expression levels or activities increase or decrease, relative to control, the dosage or administration of therapeutic agents may be adjusted.

The methods can also be used to determine the proper dosage (e.g., the therapeutically effective amount) of a therapeutic agent for the individual, the proper type of therapeutic agent, or whether a therapy should be administered.

### Methods of Screening

Provided herein are methods of screening compounds for the treatment of cancer, including: (i) contacting cancer cells or a sample derived from cancer cells with one or more test agents; (ii) detecting the expression and/or activity of MYC and MKLP2 in the cancer cells; and (iii) if the test agent modifies a cancer-dependent signature or MYC-dependent cellular phenotypic signature, identifying the test agent as a compound effective for the treatment of cancer. In some embodiments, detecting the expression and/or activity of MYC and MKLP2 in the cancer cells is performed with routine methods of detection in the art, such as those described below.

### Nucleic Acid Detection

To carry out any of the methods of the invention, a biological sample (e.g., tissue, plasma, blood, stool, or urine) can be obtained by any method known in the art. For instance, samples from an individual may be obtained by tissue biopsy (e.g., biopsy collection), aspirate (e.g., fine needle aspiration), surgical removal, skin punch, venipuncture, resection, bronchoscopy, bronchial brushings, or from stool, urine, or blood, such as serum or plasma. Genes can be detected in these samples. Samples may also include, but are not limited to, cancer cells, including cancer-derived cells, immortalized cells, or primary cells. Samples may also include, but are not limited to lung cells, breast cells, blood cells, brain cells, ovary cells, skin cells, intestine cells, pancreas cells, bone cells, kidney cells, prostate cells, stomach cells, cervix cells, or liver cells. For example, in some embodiments, the biological sample (e.g., biopsied tissue e.g., biopsied tumor tissue) is collected by biopsy collection, which may include a skin punch and/or blood processing. Following the collection of a sample, such as lung cells, breast cells, blood cells, brain cells, ovary cells, skin cells, intestine cells, pancreas cells, bone cells, kidney cells, prostate cells, stomach cells, cervix cells, or liver cells, screening of the sample can be conducted.

By screening such biological samples (e.g., biopsied tissue), a simple early diagnosis or differential diagnosis can be achieved for cancer. In addition, the progress of therapy can be monitored by testing such biological samples for the expression and/or activity of one or more (e.g., two, three, or four) genes of the cancer-dependent gene signature. Furthermore, the prediction of outcome or response to therapy can similarly be tested using such biological samples for the transcriptional profile of a cancer-dependent gene signature. For example, in some embodiments, the methods herein include detecting the expression and/or activity of one or more genes of the cancer-dependent gene signature.

Nucleic acid expression and/or activity above can be characterized using a variety of assays known to those skilled in the art. For example, a gene (e.g., MYC e.g., MYCN and/or MYCL) can be characterized by conventional assays, including but not limited to those assays described below, to determine whether it is expressed or whether its activity level is changed.

Nucleic acid-based datasets suitable for analysis in conjunction with the compositions and methods of the invention include gene expression profiles. Such profiles may include whole transcriptome sequencing data (e.g., RNA-Seq data), panels of mRNAs, noncoding RNAs, or any other nucleic acid sequence that may be expressed from genomic DNA. Other nucleic acid datasets suitable for use with the compositions and methods of the invention may include expression data collected by imaging-based techniques (e.g., Northern blotting or Southern blotting). Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Gene expression profiles to be analyzed in conjunction with evaluating the compositions described herein may include, for example, microarray data or nucleic acid sequencing data produced by a sequencing method known in the art (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLID sequencing, and nanopore sequencing platforms. Additional methods of sequencing known in the art can also be used. For instance, mRNA expression levels may be determined using RNA-Seq (e.g., as described in Mortazavi et al., Nat. Methods 5:621-628, 2008, the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Gene expression levels may be determined using microarray-based platforms, as microarray technology offers high resolution. Details of various microarray methods can be found in the literature. See, for example, U.S. Pat. No. 6,232,068 and Pollack et al., Nat. Genet. 23:41-46, 1999, the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and includes arrays fabricated by direct synthesis of oligonucleotides on a glass surface. Other systems may be used as known to one skilled in the art.

Amplification-based assays also can be used to measure the expression level of one or more markers (e.g., genes). In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001, 1996, and in Heid et al., Genome Res. 6:986-994, 1996, the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

In some embodiments, the method includes sequencing RNA. Any suitable RNA sequencing method may be used, such as, for example, mRNA-Seq, total RNA-Seq, strand-specific RNA-Seq, small RNA-Seq, ultra-low input RNA-Seq, single-cell RNA-Seq, and Iso-Seq. RNA used for sequencing may be derived from a biological sample. In some embodiments, RNA is derived from a biological sample (e.g., tissue, plasma, blood, stool, or urine).

In some embodiments, the method further includes prior to determining the expression or activity level, extracting mRNA from the biological sample (e.g., tissue, plasma, blood, stool, or urine) and reverse transcribing the mRNA into cDNA to obtain a treated biological sample (e.g., tissue, plasma, blood, stool, or urine).

In certain embodiments, the mRNA level is determined by an amplification-based assay (e.g., PCR, quantitative PCR, or real-time quantitative PCR), amplification-free assay (e.g., Nanostring), microdroplet based assay, nanopore based assay, or bead based assays (e.g., Luminex, nanoparticles, Nanosphere).

Next generation sequencing methods may also be used with the methods of the invention. Next generation sequencing methods are sequencing technologies that parallelize the sequencing process, producing thousands or millions of sequences concurrently (see, for example, Hall, J. Exp. Biol. 209 (Pt.9): 1518-1525 (2007) for a review of next generation methods). Next generation sequencing methods include, but are not limited to, polony sequencing, 454 pyrosequencing, Illumina (Solexa) sequencing, SOLID sequencing, Ion Torrent semiconductor sequencing, DNA nanoball sequencing, Heliscope single molecule sequencing, single molecule real time sequencing, nanopore DNA sequencing (see, for example, Dela Torre et al. Nanotechnology, 23 (38): 385308, 2012), tunneling currents DNA sequencing (see, for example, Massimiliano, Nanotechnology, 24:342501, 2013), sequencing by hybridization (see, for example, Qin et al. PLOS One, 7 (5): e35819, 2012), sequencing with mass spectrometry (see, for example, Edwards et al. Mutation Research, 573 (1-2): 3-12, 2005), microfluidic Sanger sequencing (see, for example, Kan et al. Electrophoresis, 25 (21-22): 3564-3588, 2004), microscopy-based sequencing (see, for example, Bell et al. Microscopy and microanalysis: the official journal of Microscopy Society of America, Microbeam Analysis Society, Microscopical Society of Canada, 18 (5): 1-5, 2012), and RNA polymerase sequencing (see, for example, Parcek et al. J. Applied Genetics, 52 (4): 413-415, 2011).

In some embodiments, the method includes sequencing RNA derived from the biological sample. For example, in some embodiments, the method incudes detecting a transcriptional profile of a one or more agent(s) that inhibit cancer cell viability and/or proliferation-induced response

In some embodiments, the method includes assessing epigenetic changes of the one or more genes of the cancer-dependent gene signature. Assessing epigenetic changes may be performed by methods known in the art, such as by a chromatin immunoprecipitation assay (CHIP), among others (for a review, see e.g., DeAngelis, J. Tyson, Woodrow J. Farrington, and Trygve O. Tollefsbol. Molecular biotechnology 38.2 (2008): 179-183, incorporated herein in its entirety by reference).

For example, DNA sequencing and the use of methylation-sensitive primers (MSPs) are two commonly used techniques to analyze bisulfite-treated DNA for assessing epigenetic changes, as bisulfite modification of DNA enables the analysis of changes in methylation patterns. The differences in bisulfite-based methylation assays arise from the manner in which bisulfite-modified DNA is analyzed. Bisulfite modification converts nonmethylated cytosines to uracils, which are then converted to thymines during DNA amplification by PCR, whereas methylated cytosines are protected from bisulfite modification. Sequencing analysis of bisulfite-modified DNA can be used to reveal the methylation status of specific cytosines, whereas MSPs can be used to quickly assess a large number of CpG islands.

Additionally, single nucleotide primer extension (SNuPE) provide yet another means to analyze bisulfite-modified DNA. The extension of an oligonucleotide to the 5′ end of a CpG site using dideoxycytidines (ddCTP) or dideoxythymidine (ddTTP) followed by real-time PCR, allows for a quantitative assessment of methylation patterns and can be applied to multiple sites simultaneously. A semi-quantitative method known as methylation sensitive-single strand conformation analysis (MS-SSCA) can also be used to obtain an overall picture of DNA methylation. MS-SSCA can be applied across a broad range of samples and can be used to assess the ratio of methylated to nonmethylated DNA.

Digestion of genomic DNA with endonucleases that differ in their methylation sensitivities is yet another method for obtaining a rough estimate of the totality of methylation.

Alternatively, for example, there are many additional techniques to analyze changes in DNA methylation, with the optimal method depending on factors including, but not limited to, the availability of the DNA, total number of targets being analyzed, or the desired specificity. One assay to assess a large number of CpG islands is restriction landmark genomic scanning (RLGS). This method involves the radioactive labeling of nonmethylated sequences that are targets of methylation sensitive restriction enzymes

The ChIP assay, which assesses changes in chromatin structure, comprises one of the most utilized assays in epigenetic research. ChIP assays monitor DNA-protein interactions and allow the chromatin structure surrounding a specific DNA sequence to be analyzed. A conventional ChIP (xChIP) uses formaldehyde to crosslink DNA and protein, followed by immunoprecipitation of DNA-protein complexes. Once the crosslinks are reversed, recovered DNA can then be analyzed using PCR. Another commonly used form of the ChIP assay is the native ChIP (nChIP). nChIP uses micrococcal nuclease digestion to prepare the chromatin for analysis. nChIP allows for modifications of histones, such as methylation or acetylation, to be assessed more accurately than with formaldehyde fixation; however, nChIP does not usually allow for assessment of proteins with a weak binding affinity for DNA. Most ChIP assays are semi-quantitative, although combining either ChIP assay with real-time PCR (Q-ChIP) can achieve a quantitative measurement of the amount of DNA bound to a specific protein.

ChIP assays can also be combined with other epigenetic assays such as DNA bisulfite modification. DNA harvested from a ChIP assay can be treated with bisulfite, while MSPs can be used to assess changes in DNA methylation in a ChIP-MSP. Other useful techniques to assess genome-wide epigenetic changes includes the ChIP-on-Chip assay that utilizes traditional ChIP protocols combined with microarray analysis.

In addition to ChIP, many other assays exist that can be used to assess chromatin structure. For example, DNaseI hypersensitivity assays can be used if a more general determination of the changes chromatin has undergone is desired. DNaseI hypersensitivity sites are usually located in or around promoter regions thereby allowing for mapping of transcriptionally active versus inactive chromatin. One useful technique to assess changes in chromatin structure is the use of the deacetylating agent trichostatin A (TSA).

In some embodiments, RNA is derived from cells. In some embodiments, the cells are lung cells, breast cells, blood cells, brain cells, ovary cells, skin cells, intestine cells, pancreas cells, bone cells, kidney cells, prostate cells, stomach cells, cervix cells, or liver cells.

In some embodiments, the methods include detecting MYC expression. In some embodiments detecting MYC expression includes detecting MYCN and/or MYCL.

In some embodiments, detecting includes detecting a transcriptional profile of a cancer-dependent signature. In some embodiments, detecting the transcriptional profile of a cancer-dependent signature includes detecting expression of MYC and MKLP2, wherein detecting expression of MYC and MKLP2 includes sequencing RNA derived from a biological sample or cancer cells.

In some embodiments, the methods herein include detecting an abnormality in the MYC gene and/or the MKLP2 gene. For example, in some embodiments, the method includes detecting an abnormality in the MYC gene. In some embodiments, the method includes detecting an abnormality in the MKLP2 gene.

In some embodiments, detecting the abnormality in the MYC gene includes detecting a mutation in MYC, a translocation of MYC, a copy number of MYC, or combinations thereof. For example, in some embodiments, detecting the abnormality in the MYC gene includes detecting a mutation in MYC. In some embodiments, detecting the abnormality in the MYC gene includes detecting a translocation of MYC. In some embodiments, detecting the abnormality in the MYC gene includes detecting a copy number of MYC.

In some embodiments, detecting the abnormality in the MKLP2 gene includes detecting a mutation in MKLP2, a translocation of MKLP2, a copy number of MKLP2, or combinations thereof. For example, in some embodiments, detecting the abnormality in the MKLP2 gene includes detecting a mutation in MKLP2. In some embodiments, detecting the abnormality in the MKLP2 gene includes detecting a translocation of MKLP2. In some embodiments, detecting the abnormality in the MKLP2 gene includes detecting a copy number of MKLP2.

Also provided herein are kits for detecting expression of MYC and MKLP2 in a biological sample obtained from an individual who may benefit from a treatment including one or more agent(s) that inhibit cancer cell viability and/or proliferation (e.g., LC30, LW33R, LXY013, LXY018, LC02, LC09, LG157, LG160, LG169, LG171, LG172, LG177, and LG181), the kit including: (i) at least a first and a second test agent, wherein the first test agent detects MYC expression (e.g., RNA expression) and the second test agent detects MKLP2 expression (e.g., RNA expression); and (ii) instructions for use.

### Protein Detection

To carry out any of the methods of the invention, a biological sample (e.g., tissue, plasma, blood, stool, or urine) can be obtained by any method known in the art. For instance, samples from an individual may be obtained by tissue biopsy (e.g., biopsy collection), aspirate (e.g., fine needle aspiration), surgical removal, skin punch, venipuncture, resection, bronchoscopy, bronchial brushings, or from stool, urine, or blood, such as serum or plasma. Proteins can be detected in these samples. Samples may also include, but are not limited to, cancer cells, including cancer-derived cells, immortalized cells, or primary cells. Samples may also include, but are not limited to lung cells, breast cells, blood cells, brain cells, ovary cells, skin cells, intestine cells, pancreas cells, bone cells, kidney cells, prostate cells, stomach cells, cervix cells, or liver cells. For example, in some embodiments, the biological sample (e.g., biopsied tissue e.g., biopsied tumor tissue) is collected by biopsy collection, which may include a skin punch and/or blood processing. Following the collection of a sample, such as lung cells, breast cells, blood cells, brain cells, ovary cells, skin cells, intestine cells, pancreas cells, bone cells, kidney cells, prostate cells, stomach cells, cervix cells, or liver cells, screening of the sample can be conducted.

Gene expression can additionally be determined by measuring the concentration or relative abundance of a corresponding protein product encoded by a gene of interest. Protein levels can be assessed using standard detection techniques known in the art. Examples of protein expression analysis that generate data suitable for use with the methods described herein include, without limitation, proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. For example, in some embodiments, quantification includes quantifying an image obtained from an immunohistochemistry assay, a FISH assay, an RNA-seq, a Taqman, quantitative PCR, a proteomics assay, an immunoblot, or an ELISA. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

For example, in some embodiments, the sample may be contacted with an antibody specific for the target protein (e.g., MYC or MKLP2) under conditions sufficient for an antibody-protein complex to form, and detection of the complex. The presence of the biomarker may be detected in a number of ways, such as by immunoblotting or ELISA procedures using any of a wide variety of tissues or samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the noncompetitive types, as well as traditional competitive binding assays. These assays also include direct binding of a labeled antibody to a target biomarker.

For example, provided herein, in some embodiments is a method of detecting expression and/or activity of MYC and MKLP2, including: contacting a biological sample obtained from an individual diagnosed with cancer or experiencing one or more symptoms associated with cancer with at least a first and second test agent; wherein the first test agent detects MYC expression and/or activity and the second test agent detects MKLP2 expression and/or activity. In one embodiment, the method further includes detecting expression of MYC and/or MKLP2. In some embodiments, the first test agent includes an antibody that binds MYC. In some embodiments, the second test agent includes an antibody that binds MKLP2. Using such a method, an individual is identified as an individual who may benefit from a treatment comprising one or more agent(s) that inhibit cancer cell viability and/or proliferation if expression of MYC is elevated relative to a suitable control and expression of MKLP2 is decreased relative to a suitable control (e.g., expression of β-Actin in the same biological sample).

Another method involves immobilizing the target biomarkers (e.g., on a solid support) and then exposing the immobilized target to a specific antibody, which may or may not contain a label. Depending on the amount of target and the strength of the label's signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by a label, e.g., an enzyme, a fluorescent label, a chromogenic label, a radionuclide containing molecule (i.e., a radioisotope), or a chemiluminescent molecule.

Variations on the forward assay include a simultaneous assay, in which both sample and labeled antibody are added simultaneously to a bound antibody. These techniques are well known to those skilled in the art, including any minor variations as will be readily apparent. In a typical forward sandwich assay, a first antibody having specificity for the biomarker is either covalently or passively bound to a solid surface (e.g., a glass or a polymer surface, such as those with solid supports in the form of tubes, beads, discs, or microplates), and a second antibody is linked to a label that is used to indicate the binding of the second antibody to the molecular marker.

In alternative methods, the expression of a protein in a sample may be examined using immunohistochemistry (“IHC”) and staining protocols. IHC staining of tissue sections has been shown to be a reliable method of assessing or detecting presence of proteins in a sample. IHC and immunofluorescence techniques use an antibody to probe and visualize cellular antigens in situ, generally by chromogenic or fluorescent methods. The tissue sample may be fixed (i.e., preserved) by conventional methodology (see, e.g., “Manual of Histological Staining Method of the Armed Forces Institute of Pathology,” 3rd edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. By way of example, neutral buffered formalin, Bouin's or formaldehyde, may be used to fix a sample. Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. The primary and/or secondary antibody used for immunohistochemistry typically will be labeled with a detectable moiety, such as a radioisotope, a colloidal gold particle, a fluorescent label, a chromogenic label, or an enzyme-substrate label.

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including but not limited to monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Alternatively, the levels of biomarkers may be detected without the use of binding agents. In some instances, biological sample (e.g., tissue, plasma, blood, stool, or urine) as described herein are analyzed, for example by one or more, enzymatic methods, chromatographic methods, mass spectrometry (MS) methods, chromatographic methods followed by MS, electrophoretic methods, electrophoretic methods followed by MS, nuclear magnetic resonance (NMR) methods, and combinations thereof. In some instances, the biological sample (e.g., tissue, plasma, blood, stool, or urine) is treated with one or more enzymes (e.g., trypsin). Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (U PLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), Multiple Reaction Monitoring (MRM), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by immunoblotting using antibodies that recognize specific glycan structures, and combinations thereof. Exemplary nuclear magnetic resonance (NMR) include, but are not limited to, one-dimensional NMR (1 D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NM R), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

Mass spectrometry (MS) may be used in conjunction with the methods described herein to identify and characterize the gene expression profile of a single cell or multi-cell population. Any method of MS known in the art may be used to determine, detect, and/or measure a peptide or peptides of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g. by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for methods described herein allowing for ionization followed by fragmentation of a complex peptide sample, such as a sample obtained from a multi-cell population described herein. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example by searching spectra against a database while allowing for specific peptide modifications.

Any of the methods herein that rely upon protein measurement can also be adapted for use with the measurement of mRNA levels for the protein. The level of mRNA can be determined using methods known in the art. Methods to measure mRNA levels generally include, but are not limited to, sequencing, northern blotting, RT-PCR, gene array technology, and RNAse protection assays, as described above.

In some embodiments, the methods include detecting MYC expression and/or activity. In some embodiments detecting MYC expression includes detecting MYCN and/or MYCL.

In some embodiments detecting includes detecting a MYC-dependent cellular phenotypic signature, which may be categorized by protein expression.

Also provided herein are kits for detecting expression of MYC and MKLP2 in a biological sample obtained from an individual who may benefit from a treatment including one or more agent(s) that inhibit cancer cell viability and/or proliferation (e.g., LC30, LW33R, LXY013, LXY018, LC02, LC09, LG157, LG160, LG169, LG171, LG172, LG177, and LG181), the kit including: (i) at least a first and a second test agent, wherein the first test agent detects MYC expression (e.g., protein expression and/or activity) and the second test agent detects MKLP2 expression (e.g., protein expression and/or activity); and (ii) instructions for use.

### Image-Based Assays

To carry out the methods of the invention, in some embodiments, a MYC-dependent cellular phenotypic signature is detected, such as with an image-based screening assay (e.g., an immunofluorescences assay, phase-contrast microscopy, fluorescent microscopy, brightfield microscopy, confocal microscopy, 4D live-cell imaging e.g., time-lapse microscopy, and automated microscopy), a 3-(4,5-dimethylthiazol-2-yl)-2,5-diphenyltetrazolium bromide tetrazolium (MTT) assay, a CellTiter-Glo® (CTG) assay, or a lactate dehydrogenase (LDH) assay, or combinations thereof.

The MTT assay is a colorimetric assay for assessing cell metabolic activity based upon the premise that NAD (P) H-dependent cellular oxidoreductase enzymes may, under defined conditions, reflect the number of viable cells present. Such enzymes are capable of reducing the tetrazolium dye MTT to its insoluble formazan, which has a purple color. In some embodiments, adaptations to the MTT assay can be used. For example, other closely related tetrazolium dyes including 2,3-Bis-(2-Methoxy-4-Nitro-5-Sulfophenyl)-2H-Tetrazolium-5-Carboxanilide (XTT), 3-(4,5-dimethylthiazol-2-yl)-5-(3-carboxymethoxyphenyl)-2-(4-sulfophenyl)-2H-tetrazolium (MTS), and various water-soluble tetrazoliums (WSTs), can be used in conjunction with an intermediate electron acceptor, 1-methoxy phenazine methosulfate (PMS). With WST-1, for example, which is cell-impermeable, reduction occurs outside the cell via plasma membrane electron transport.

Also provided herein is the CTG assay, which uses ATP, a required co-factor of the luciferase reaction, as an indicator of metabolically active cells. The enzyme luciferase acts on luciferin in the presence of Mg²⁺ and ATP to produce oxyluciferin and to release energy in the form of luminescence. Since the luciferase reaction requires ATP, the luminescence produced is proportional to the amount of ATP present, an indicator of cellular metabolic activity.

Also provided herein is the LDH assay, also known as LDH release assay, which is a cell death (e.g., cytotoxicity) assay used, for example, to assess the level of plasma membrane damage in a cell population. LDH is a stable enzyme, present in many cell types, which is rapidly released into a cell culture medium, for example, upon damage of the plasma membrane. The LDH assay protocol is based on an enzymatic coupling reaction in which LDH released from a cell that oxidizes lactate to generate NADH, which then reacts with WST to generate a yellow color. In such an assay, the intensity of the generated color correlates with the number of lysed cells.

Using an image-based assay, as described herein, in some embodiments, the assay may detect mitotic arrest, induction of polyploidy, cell death, or combinations thereof.

In some embodiments of the screening methods disclosed herein, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition, and processing, etc. Certain general principles and techniques that may be applied in embodiments of a HTS of the present invention are described in Macarron R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg HOser.

Agents that Inhibit Cancer Cell Viability and/or Proliferation

In some embodiments, the methods herein include administering one or more agent(s) that inhibit cancer cell viability and/or proliferation (e.g., LC30, LW33R, LXY013, LXY018, LC02, LC09, LG157, LG160, LG169, LG171, LG172, LG177, and LG181).

In some embodiments, an agent that inhibits cancer cell viability and/or proliferation is a compound of Formula (I):

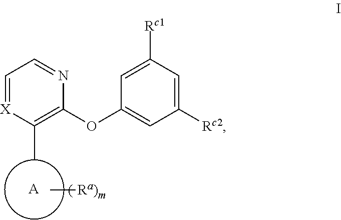

or a pharmaceutically acceptable salt,
   wherein
X is —CH═ or —N═;
Group A is optionally substituted phenyl or optionally substituted 6-membered heteroaryl;
each R^{a} is independently selected from the group consisting of halogen, —CN, —OH, —OR¹, —SR¹, —NH₂, —NR²R³, —C(O)R¹, —C(O)OH, —C(O)OR¹, —C(O)NH₂, —C(O)NR²R³, optionally substituted C₁-C₇ aliphatic, optionally substituted phenyl, optionally substituted 5-10 membered heteroaryl, and optionally substituted 5-10 membered heterocyclyl;
R^{c1} is selected from the group consisting of C₁-C₆ haloalkyl, halogen, —OR¹, —C(O)OR¹, and optionally substituted 5-6 membered heteroaryl;
R^{c2} is selected from the group consisting of halogen, —CN, —OH, —OR¹, —NH₂, —NR²R³, optionally substituted C₁-C₇ aliphatic, optionally substituted phenyl, —C(O)R¹, —C(O)OH, —C(O)OR¹, —C(O)NH₂, —C(O)NR²R³, optionally substituted 5-10 membered heteroaryl, and optionally substituted 5-10 membered heterocyclyl;
each R¹ is independently selected from the group consisting of optionally substituted C₁-C₆ aliphatic, optionally substituted phenyl, optionally substituted 5-10 membered heteroaryl, and optionally substituted 5-10 membered heterocyclyl;
each R₂ is independently selected from the group consisting of —C(O)R¹, —C(O)OR¹, —C(O)NR¹R³, optionally substituted C₁-C₆ aliphatic, optionally substituted phenyl, optionally substituted 5-10 membered heteroaryl, and optionally substituted 5-10 membered heterocyclyl;
each R³ is independently selected from the group consisting of hydrogen, —C(O)R¹, —C(O)OR¹, —C(O)NHR¹, optionally substituted C₁-C₆ aliphatic, optionally substituted phenyl, optionally substituted 5-10 membered heteroaryl, and optionally substituted 5-10 membered heterocyclyl; and
m is 0, 1, 2, 3, 4, or 5.

In some embodiments, an agent that inhibits cancer cell viability and/or proliferation is a compound of Formula (II):

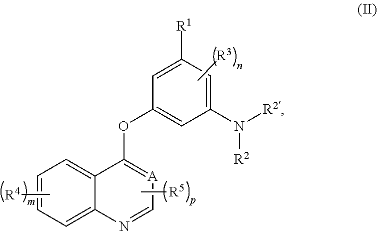

or a pharmaceutically acceptable salt or N-oxide thereof,
   wherein
A is —C(H)═ or —N═;
R¹ is selected from the group consisting of halogen, optionally substituted C₁-C₆ alkyl, optionally substituted C₁-C₆ alkoxy, optionally substituted C₁-C₆ haloalkoxy, —C(O)R^{a}, and —C(O)OR^{a};
R² is selected from the group consisting of hydrogen, optionally substituted C₁-C₆ aliphatic, optionally substituted C₁-C₆ haloaliphatic, —C(O)R^{a}, and —C(O)OR^{a};
R^{2′} is selected from the group consisting of optionally substituted C₁-C₆ aliphatic, optionally substituted C₁-C₆ haloaliphatic, —C(O)R^{a}, and —C(O)OR^{a};
optionally, R² and R^{2′} are taken together with the nitrogen on which they are attached to form optionally substituted 3-7-membered heterocyclyl or optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)ORₐ, and —C(O)N(R^{a})₂;
each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl;
each R⁵ is independently selected from the group consisting of deuterium and halogen;
each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b} and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{b} is independently optionally substituted C₁-C₆ aliphatic;
n is 0, 1, 2, or 3;
m is 0, 1, 2, 3, or 4; and
p is 0, 1, 2, or 3.

In some embodiments, an agent that inhibits cancer cell viability and/or proliferation is a compound of Formula (III):

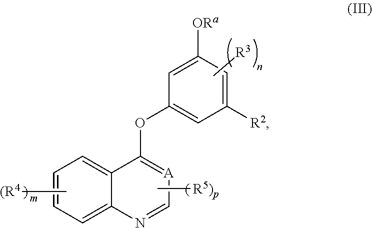

or a pharmaceutically acceptable salt or N-oxide thereof,
   wherein
A is —C(H)═ or —N═;
R² is selected from the group consisting of —NH₂, —NO₂—OR^{a}, —O(CH₂)₁₋₃R^{a}, —C(O)OR^{a}, —C(O)N(R^{a})₂, optionally substituted C₁-C₆ aliphatic, and optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;

each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl;
each R⁵ is independently selected from the group consisting of deuterium and halogen;
each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b} and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{b} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4; and
p is 0, 1, 2, or 3.

In some embodiments, an agent that inhibits cancer cell viability and/or proliferation is a compound of Formula (IV):

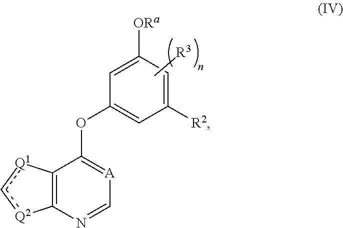

or a pharmaceutically acceptable salt or N-oxide thereof,
   wherein
A is —C(H)═ or —N═;
one of Q¹ and Q² is —N(R^{a})— or —S— and the other is —C(H)═;
R² is selected from the group consisting of —C(O)OR^{a}, —C(O)N(R^{a})₂, optionally substituted C₁-C₆ haloaliphatic, and optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R^{a} is independently is selected from the group consisting of hydrogen, optionally substituted C₁-C₆ aliphatic, optionally substituted C₁-C₆ haloaliphatic, —C(O)R^{b}, and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{b} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4.

In some embodiments an agent that inhibits cancer cell viability and/or proliferation is a compound selected from the group consisting of

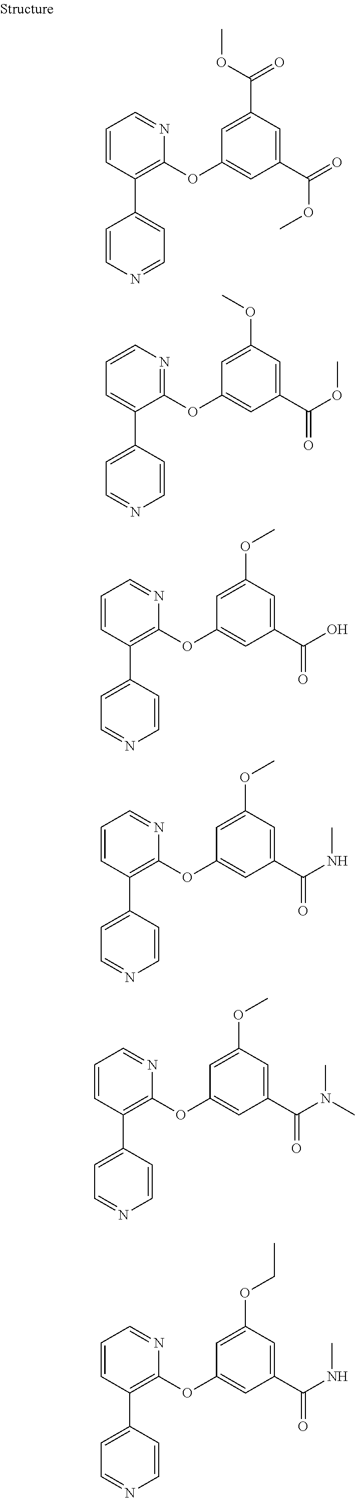 [SMILES: COC(=O)c1cc(Oc2ncccc2-c2ccncc2)cc(C(=O)OC)c1 ; COC(=O)c1cc(OC)cc(Oc2ncccc2-c2ccncc2)c1 ; COc1cc(Oc2ncccc2-c2ccncc2)cc(C(=O)O)c1 ; CNC(=O)c1cc(OC)cc(Oc2ncccc2-c2ccncc2)c1 ; COc1cc(Oc2ncccc2-c2ccncc2)cc(C(=O)N(C)C)c1 ; CCOc1cc(Oc2ncccc2-c2ccncc2)cc(C(=O)NC)c1]

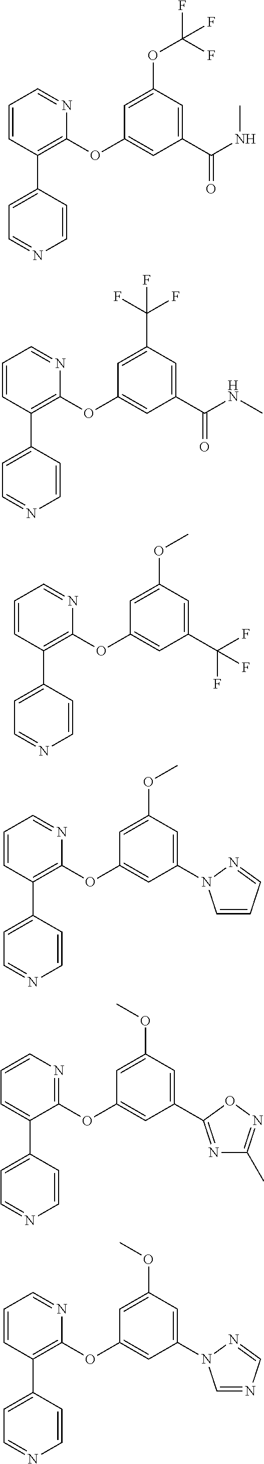 [SMILES: CNC(=O)c1cc(Oc2ncccc2-c2ccncc2)cc(OC(F)(F)F)c1 ; CNC(=O)c1cc(Oc2ncccc2-c2ccncc2)cc(C(F)(F)F)c1 ; COc1cc(Oc2ncccc2-c2ccncc2)cc(C(F)(F)F)c1 ; COc1cc(Oc2ncccc2-c2ccncc2)cc(-n2cccn2)c1 ; COc1cc(Oc2ncccc2-c2ccncc2)cc(-c2nc(C)no2)c1 ; COc1cc(Oc2ncccc2-c2ccncc2)cc(-n2cncn2)c1]

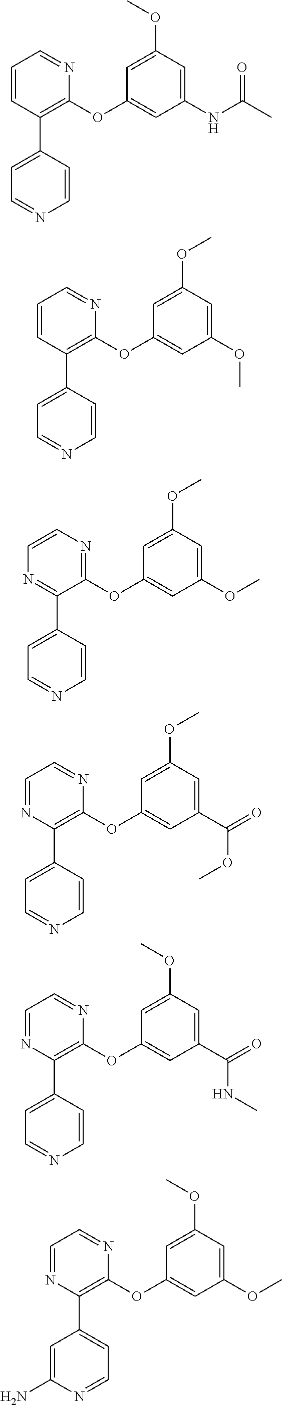 [SMILES: COc1cc(NC(C)=O)cc(Oc2ncccc2-c2ccncc2)c1 ; COc1cc(OC)cc(Oc2ncccc2-c2ccncc2)c1 ; COc1cc(OC)cc(Oc2nccnc2-c2ccncc2)c1 ; COC(=O)c1cc(OC)cc(Oc2nccnc2-c2ccncc2)c1 ; CNC(=O)c1cc(OC)cc(Oc2nccnc2-c2ccncc2)c1 ; COc1cc(OC)cc(Oc2nccnc2-c2ccnc(N)c2)c1]

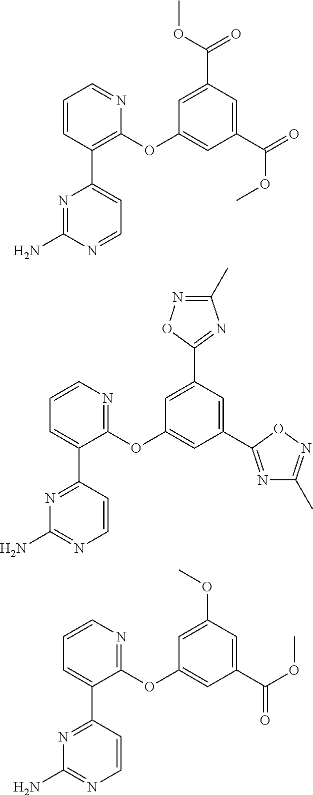 [SMILES: COC(=O)c1cc(Oc2ncccc2-c2ccnc(N)n2)cc(C(=O)OC)c1 ; Cc1noc(-c2cc(Oc3ncccc3-c3ccnc(N)n3)cc(-c3nc(C)no3)c2)n1 ; COC(=O)c1cc(OC)cc(Oc2ncccc2-c2ccnc(N)n2)c1]

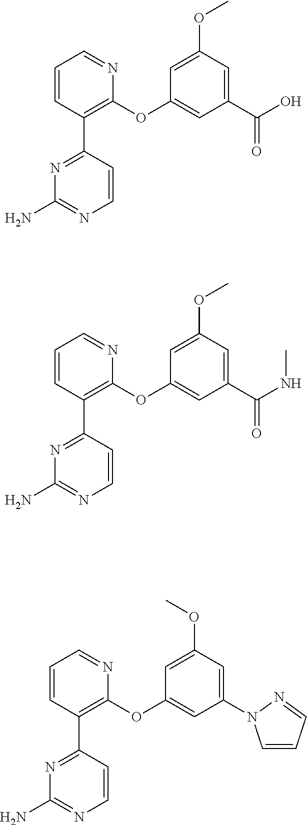 [SMILES: COc1cc(Oc2ncccc2-c2ccnc(N)n2)cc(C(=O)O)c1 ; CNC(=O)c1cc(OC)cc(Oc2ncccc2-c2ccnc(N)n2)c1 ; COc1cc(Oc2ncccc2-c2ccnc(N)n2)cc(-n2cccn2)c1]

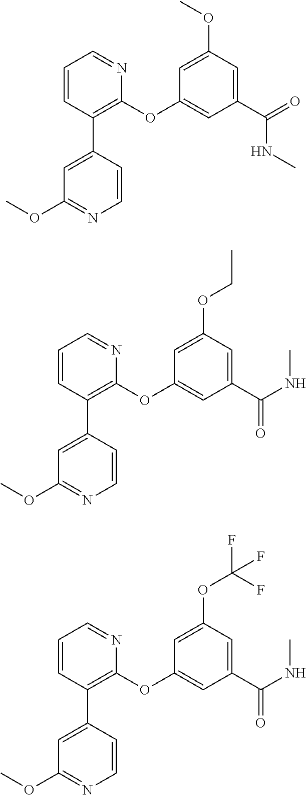

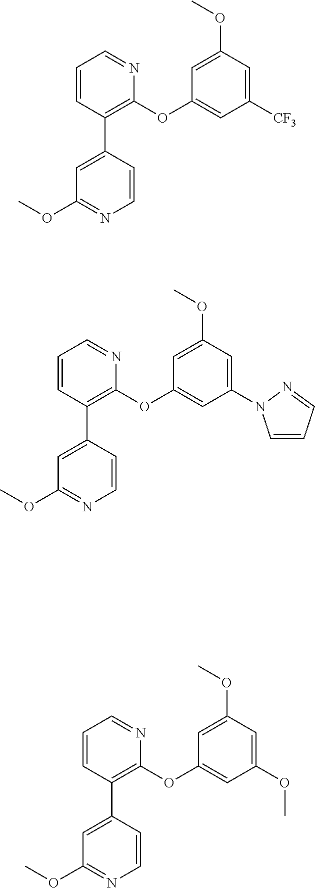

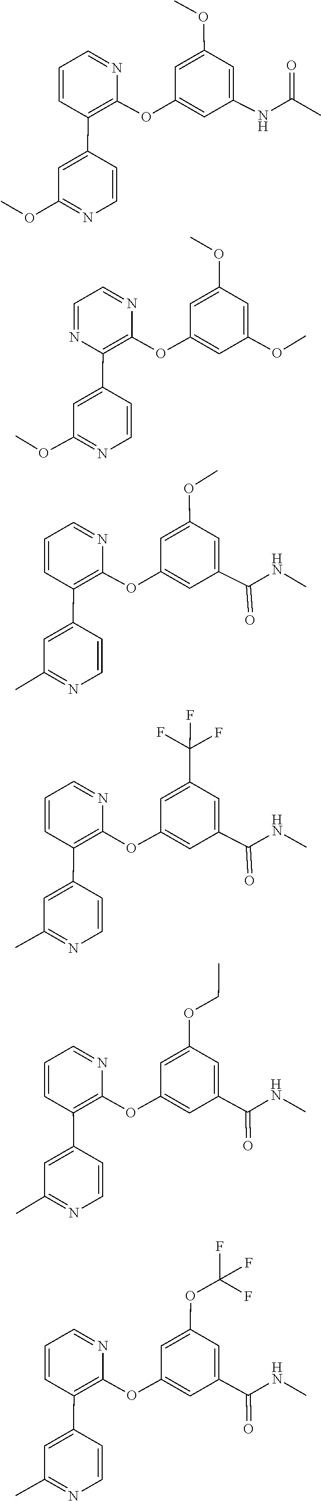

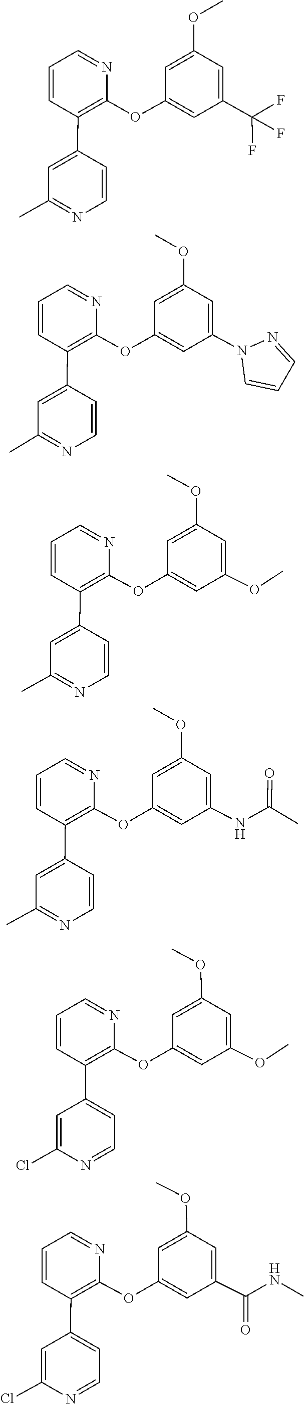

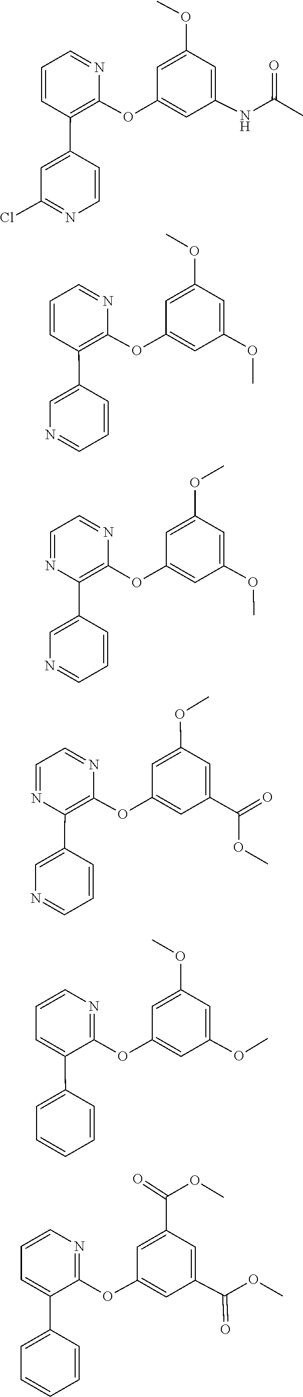 [SMILES: COc1cc(NC(C)=O)cc(Oc2ncccc2-c2ccnc(Cl)c2)c1 ; COc1cc(OC)cc(Oc2ncccc2-c2cccnc2)c1 ; COc1cc(OC)cc(Oc2nccnc2-c2cccnc2)c1 ; COC(=O)c1cc(OC)cc(Oc2nccnc2-c2cccnc2)c1 ; COc1cc(OC)cc(Oc2ncccc2-c2ccccc2)c1 ; COC(=O)c1cc(Oc2ncccc2-c2ccccc2)cc(C(=O)OC)c1]

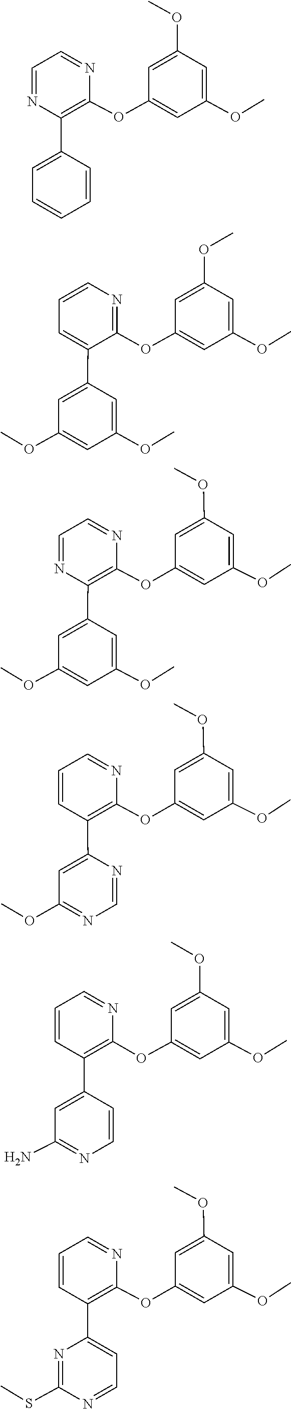

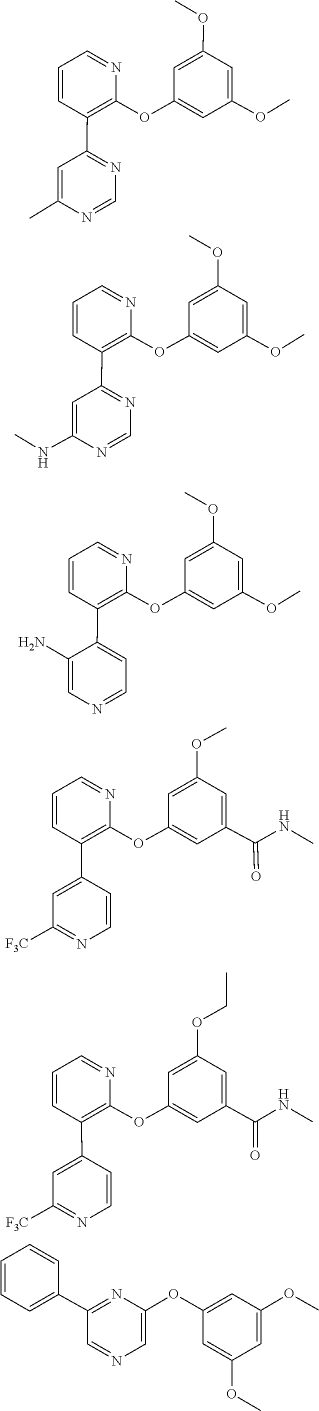

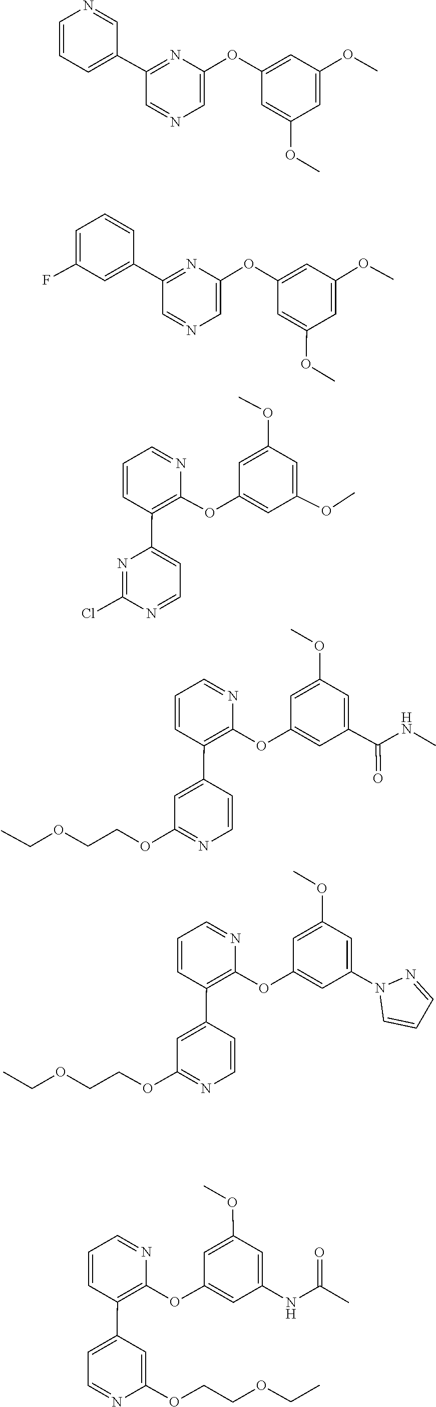

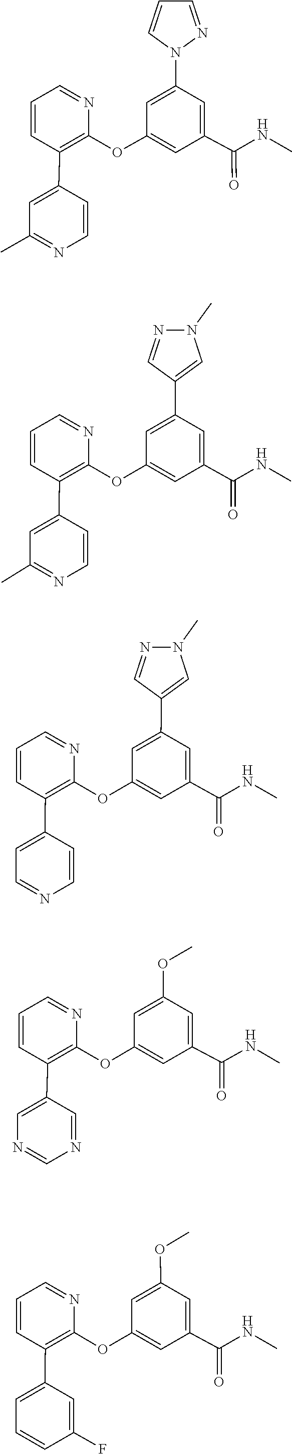

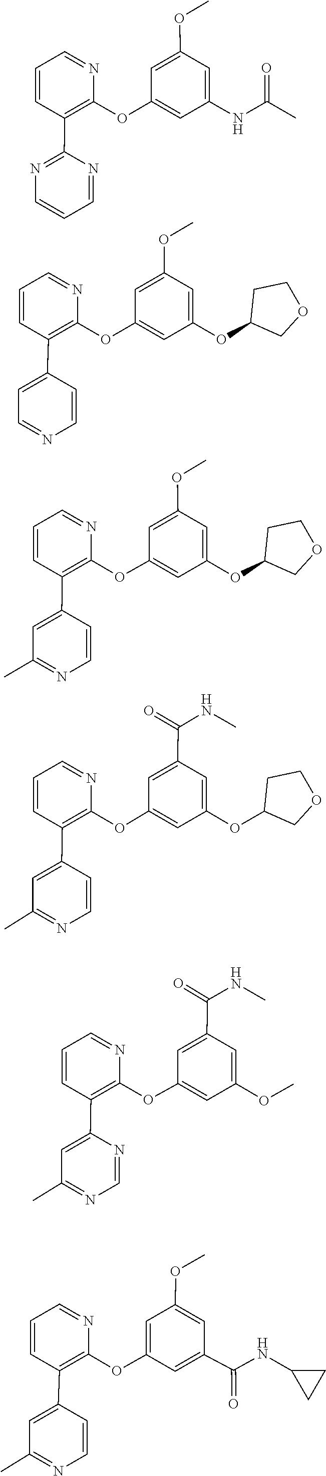

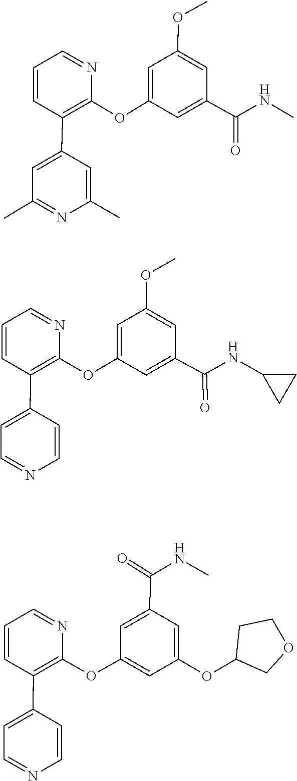

or a pharmaceutically acceptable salt thereof.

In some embodiments an agent that inhibits cancer cell viability and/or proliferation is a compound selected from the group consisting of

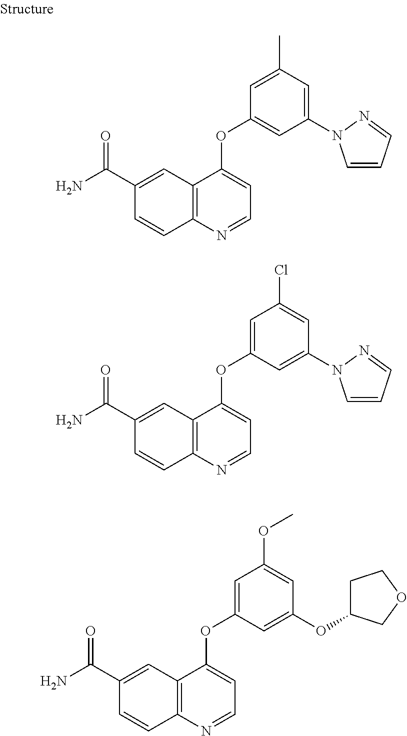 [SMILES: Cc1cc(Oc2ccnc3ccc(C(N)=O)cc23)cc(-n2cccn2)c1 ; NC(=O)c1ccc2nccc(Oc3cc(Cl)cc(-n4cccn4)c3)c2c1 ; COc1cc(Oc2ccnc3ccc(C(N)=O)cc23)cc(O[C@@H]2CCOC2)c1]

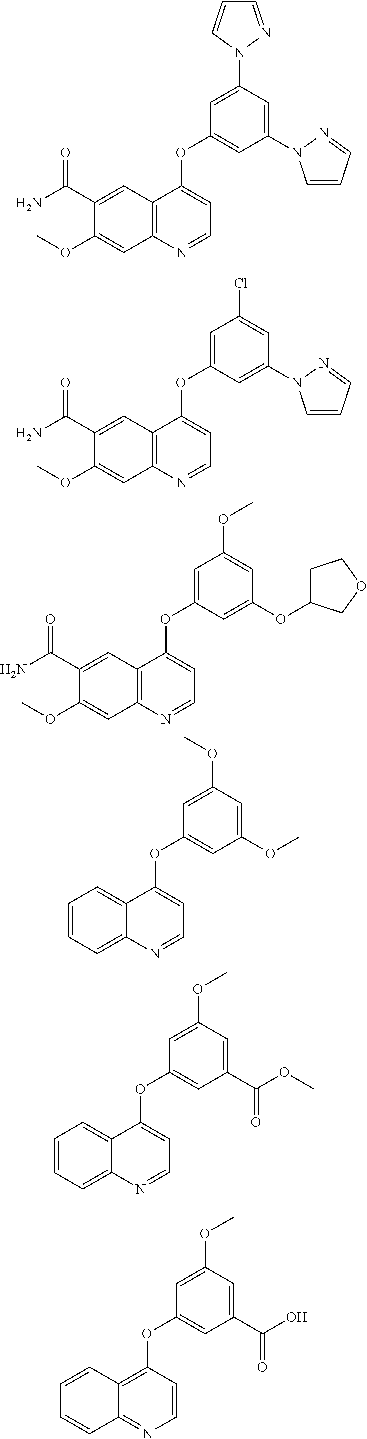

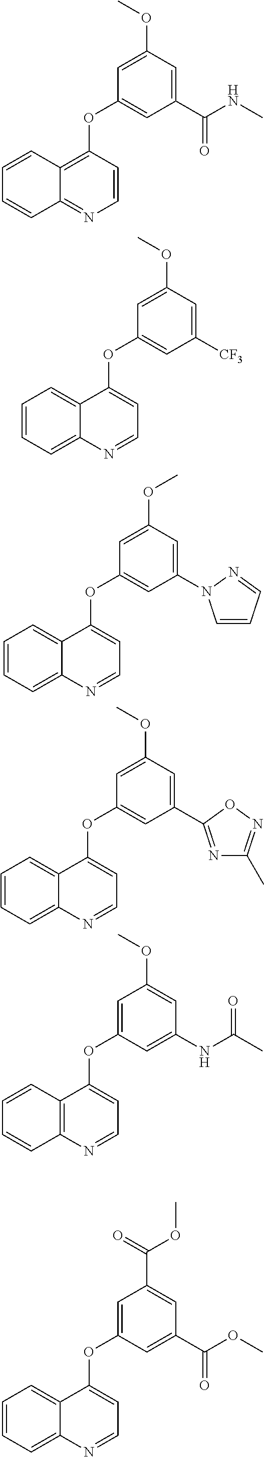

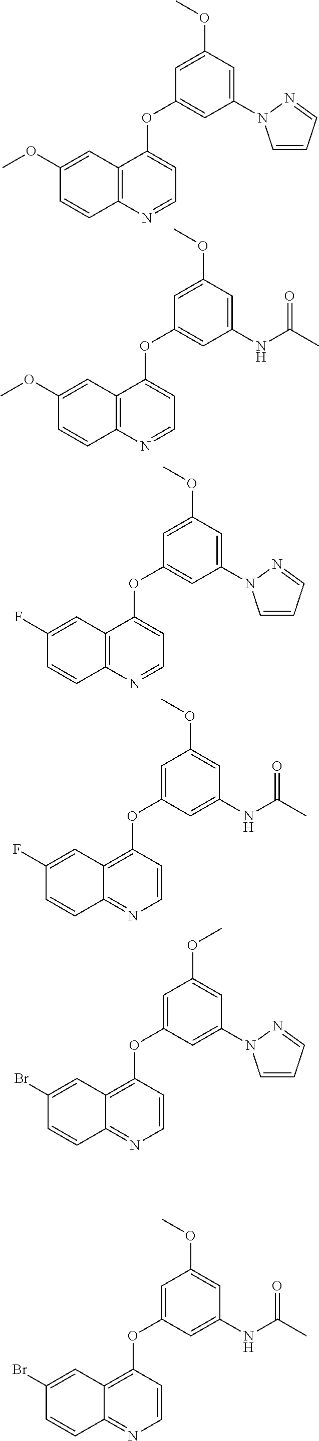

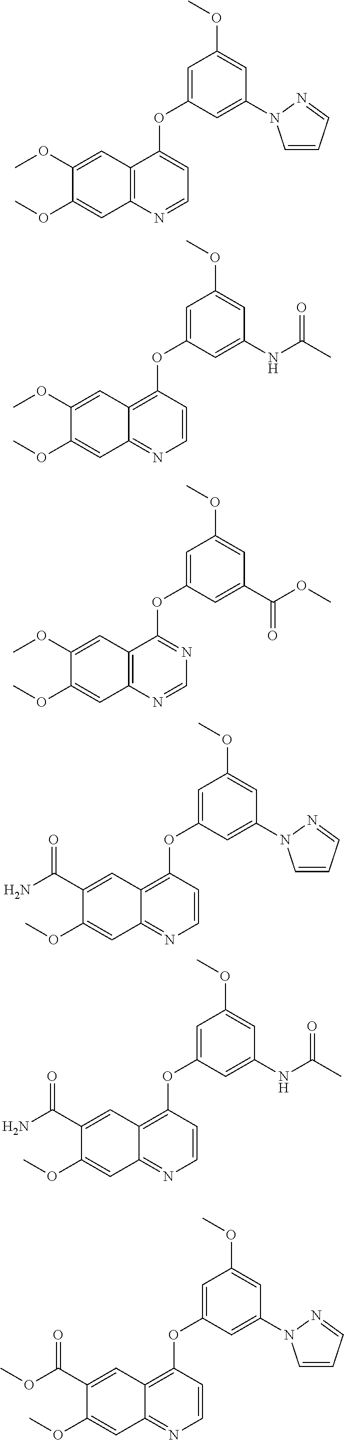

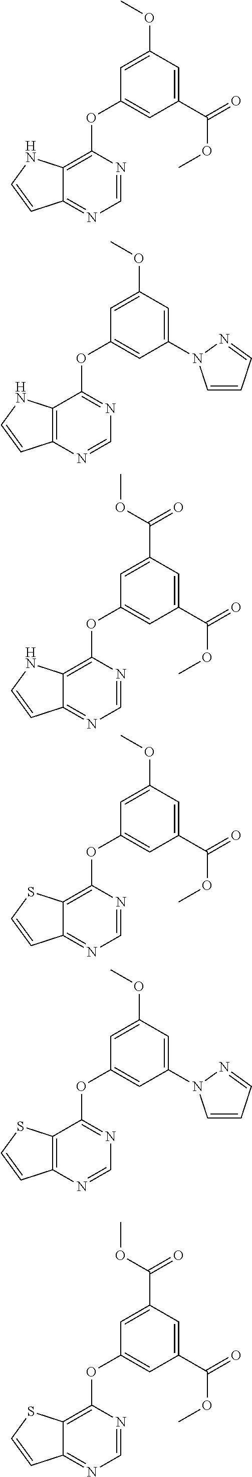

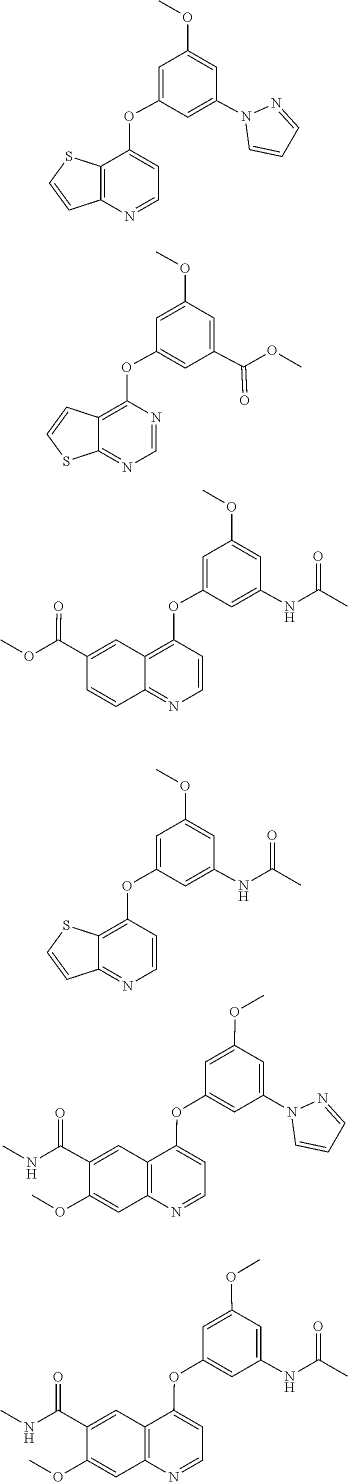

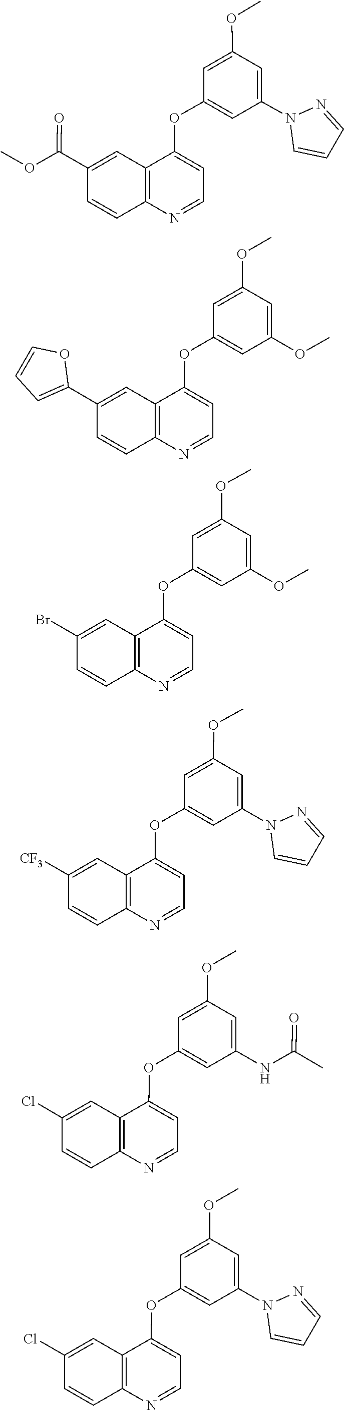 [SMILES: COC(=O)c1ccc2nccc(Oc3cc(OC)cc(-n4cccn4)c3)c2c1 ; COc1cc(OC)cc(Oc2ccnc3ccc(-c4ccco4)cc23)c1 ; COc1cc(OC)cc(Oc2ccnc3ccc(Br)cc23)c1 ; COc1cc(Oc2ccnc3ccc(C(F)(F)F)cc23)cc(-n2cccn2)c1 ; COc1cc(NC(C)=O)cc(Oc2ccnc3ccc(Cl)cc23)c1 ; COc1cc(Oc2ccnc3ccc(Cl)cc23)cc(-n2cccn2)c1]

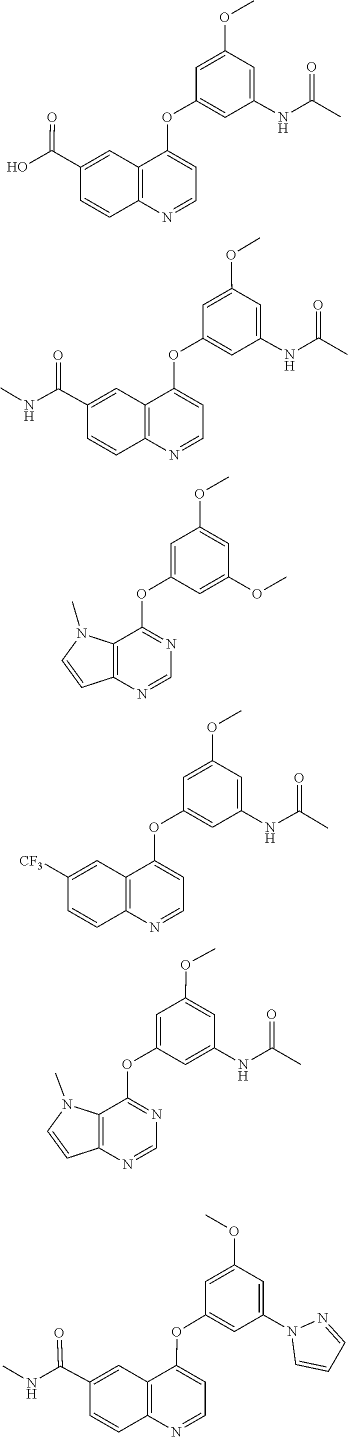

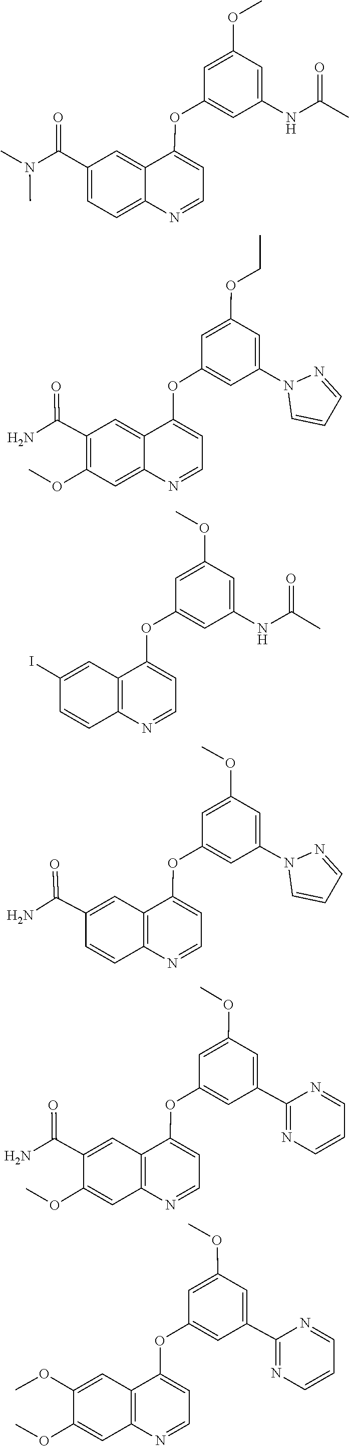

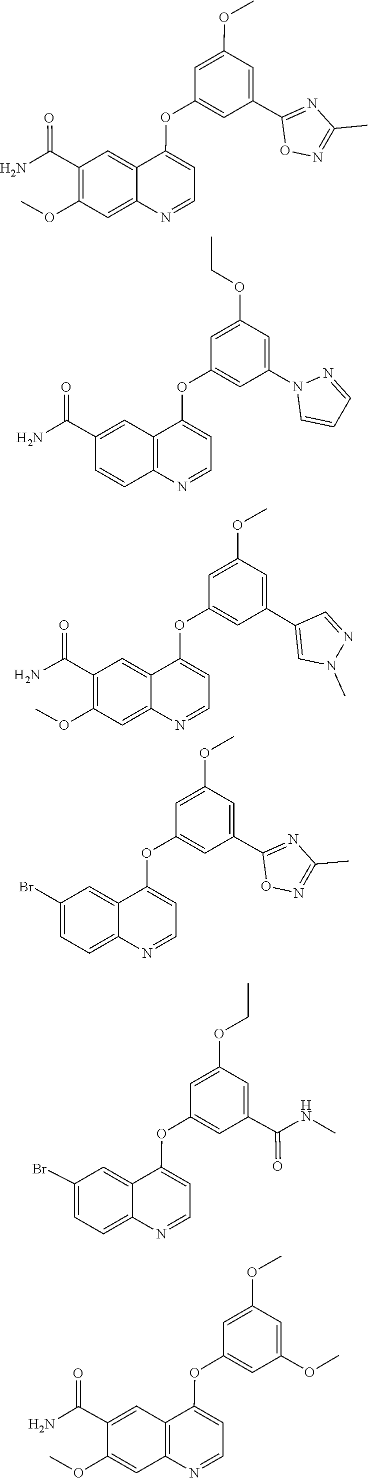

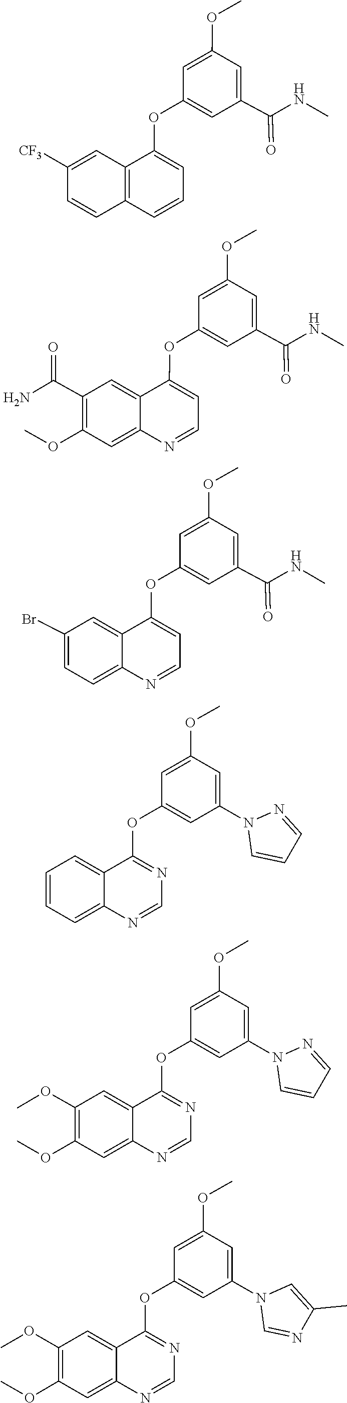

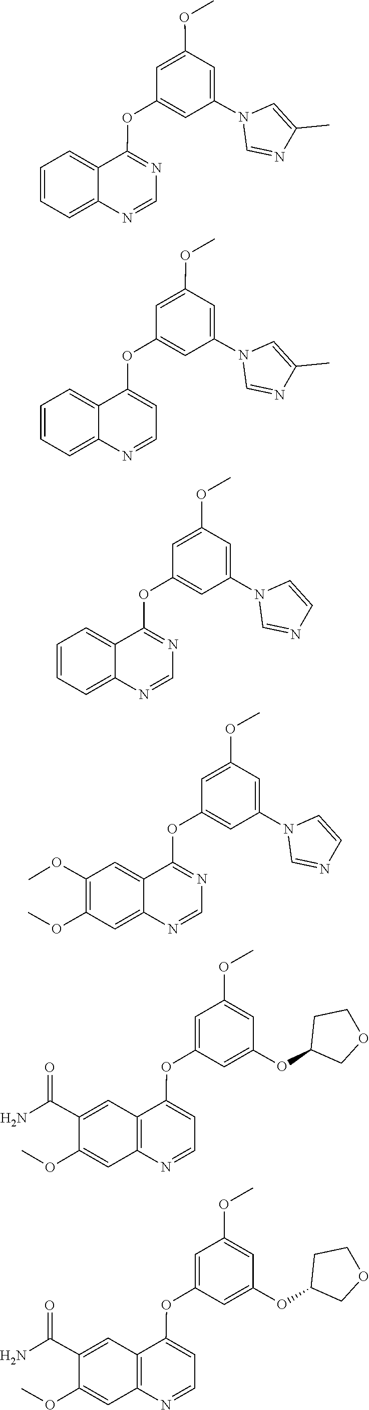

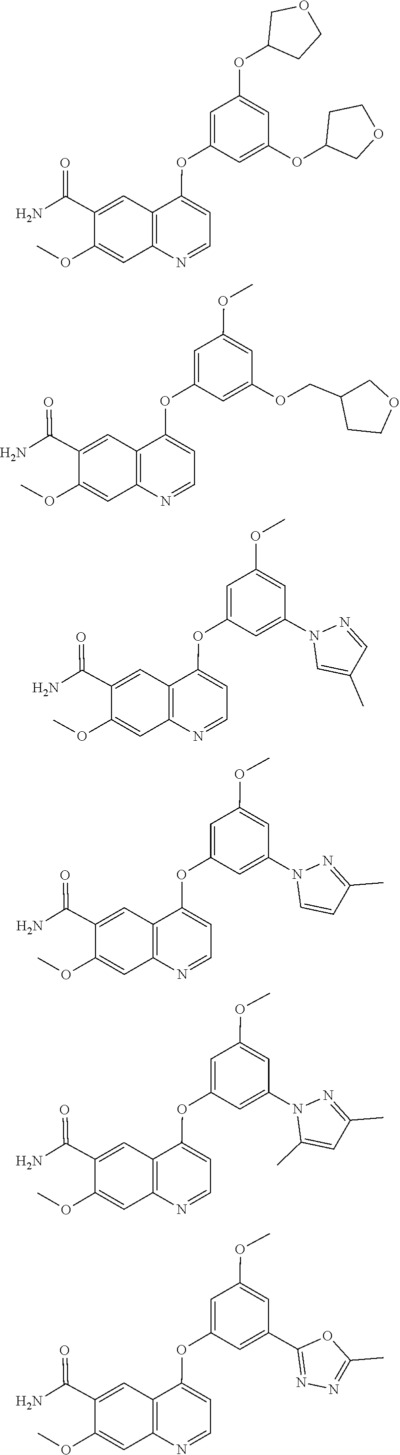

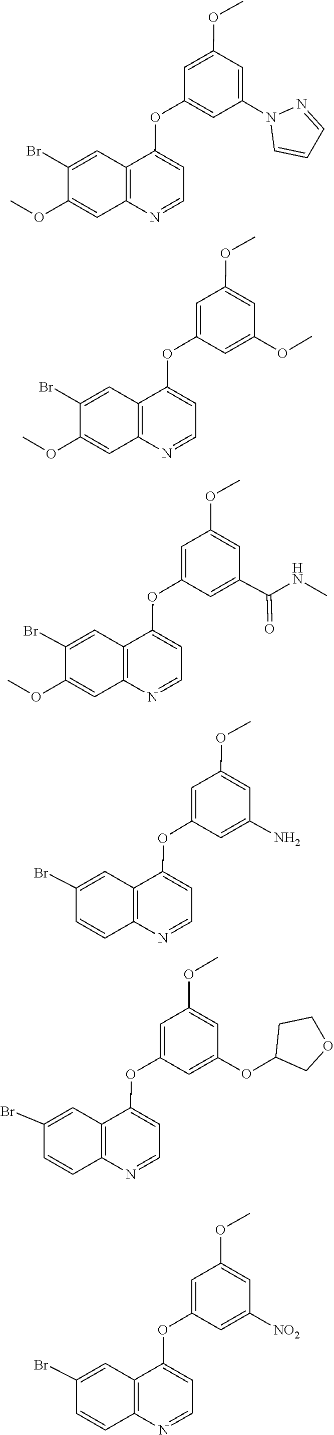

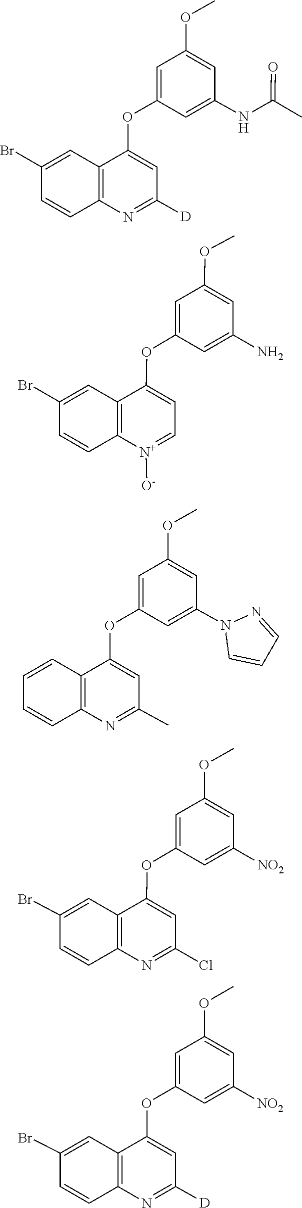

or a pharmaceutical acceptable salt thereof.

In some embodiments, the method includes administering LC30. In some embodiments, the method includes administering LW33R. In some embodiments, the method includes administering LXY013. In some embodiments, the method includes administering LXY018. In some embodiments, the method includes administering Alisertib. In some embodiments, the method includes administering LC02. In some embodiments, the method includes administering LC09. In some embodiments, the method includes administering LG157. In some embodiments, the method includes administering LC09. In some embodiments, the method includes administering LG160. In some embodiments, the method includes administering LC09. In some embodiments, the method includes administering LG169. In some embodiments, the method includes administering LC09. In some embodiments, the method includes administering LG171. In some embodiments, the method includes administering LC09. In some embodiments, the method includes administering LG172. In some embodiments, the method includes administering LC09. In some embodiments, the method includes administering LG177. In some embodiments, the method includes administering LC09. In some embodiments, the method includes administering LG181.

As used herein, LG160 refers to a compound having the structure, below:

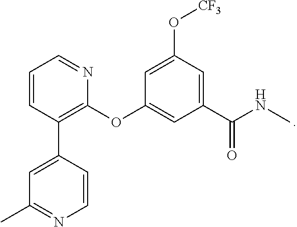

LG160 can be synthesized as described in International Publication number WO 2022/199654, incorporated herein by reference in its entirety.

As used herein, LG169 refers to a compound having the structure, below:

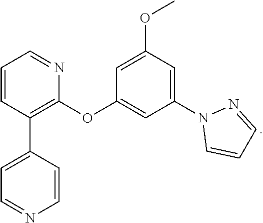 [SMILES: COc1cc(Oc2ncccc2-c2ccncc2)cc(-n2cccn2)c1]

LG169 can be synthesized as described in International Publication number WO 2022/199654, incorporated herein by reference in its entirety.

As used herein, LG171 refers to a compound having the structure, below:

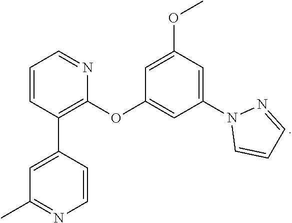 [SMILES: COc1cc(Oc2ncccc2-c2ccnc(C)c2)cc(-n2cccn2)c1]

LG171 can be synthesized as described in International Publication number WO 2022/199654, incorporated herein by reference in its entirety.

As used herein, LG172 refers to a compound having the structure, below:

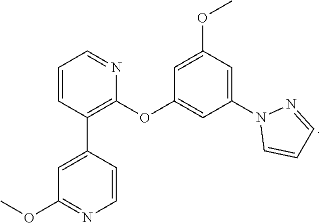 [SMILES: COc1cc(Oc2ncccc2-c2ccnc(OC)c2)cc(-n2cccn2)c1]

LG172 can be synthesized as described in International Publication number WO 2022/199654, incorporated herein by reference in its entirety.

As used herein, LG177 refers to a compound having the structure, below:

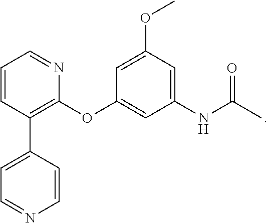 [SMILES: COc1cc(NC(C)=O)cc(Oc2ncccc2-c2ccncc2)c1]

LG177 can be synthesized as described in International Publication number WO 2022/199654, incorporated herein by reference in its entirety.

As used herein, LG181 refers to a compound having the structure, below:

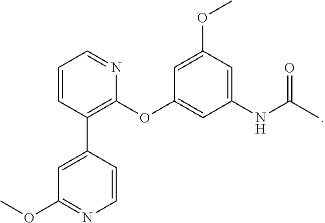 [SMILES: COc1cc(NC(C)=O)cc(Oc2ncccc2-c2ccnc(OC)c2)c1]

LG181 can be synthesized as described in Example 9.

As used herein, LG157 refers to a compound having the structure, below:

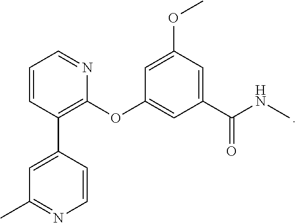

LG157 can be synthesized as described in International Publication number WO 2022/199654, incorporated herein by reference in its entirety.

As used herein, LXY013 refers to a compound having the structure, below:

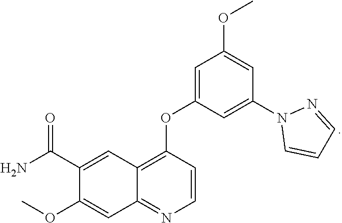 [SMILES: COc1cc(Oc2ccnc3cc(OC)c(C(N)=O)cc23)cc(-n2cccn2)c1]

LXY013 can be synthesized as described in International Publication number WO 2022/228549, incorporated herein by reference in its entirety.

As used herein, LXY018 refers to a compound having the structure, below:

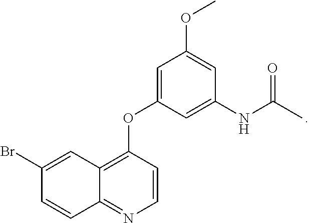 [SMILES: COc1cc(NC(C)=O)cc(Oc2ccnc3ccc(Br)cc23)c1]

LXY018 can be synthesized as described in International Publication number WO 2022/228549, incorporated herein by reference in its entirety.

As used herein, LC02 refers to a compound having the structure, below:

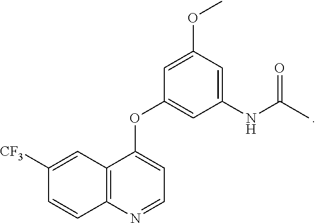 [SMILES: COc1cc(NC(C)=O)cc(Oc2ccnc3ccc(C(F)(F)F)cc23)c1]

LC02 can be synthesized as described in International Publication number WO 2022/228549, incorporated herein by reference in its entirety.

As used herein, LC09 refers to a compound having the structure, below:

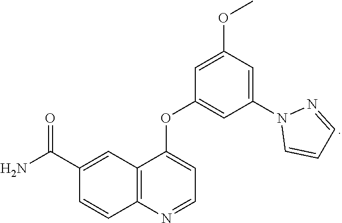 [SMILES: COc1cc(Oc2ccnc3ccc(C(N)=O)cc23)cc(-n2cccn2)c1]

LC09 can be synthesized as described in International Publication number WO 2022/228549, incorporated herein by reference in its entirety.

As used herein, LC30 refers to a compound having the structure, below:

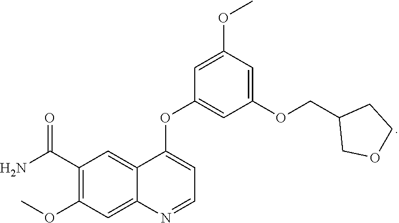 [SMILES: COc1cc(OCC2CCOC2)cc(Oc2ccnc3cc(OC)c(C(N)=O)cc23)c1]

LC30 can be synthesized as described in International Publication number WO 2022/228549, incorporated herein by reference in its entirety.

As used herein, LW33R refers to a compound having the structure, below:

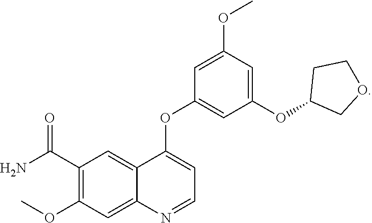 [SMILES: COc1cc(Oc2ccnc3cc(OC)c(C(N)=O)cc23)cc(O[C@@H]2CCOC2)c1]

LW33R can be synthesized as described in International Publication number WO 2022/228549, incorporated herein by reference in its entirety.

In some embodiments, the one or more agent(s) that inhibit cancer cell viability and/or proliferation includes an agent that dysregulates MYC, one or more mitotic kinase(s), one or more mitotic motor protein(s), and/or an upstream regulator or a downstream effector of CPPC. For example, in some embodiments, the one or more agent(s) that inhibit cancer cell viability and/or proliferation includes an agent that dysregulates MYC. In some embodiments, the one or more agent(s) that inhibit cancer cell viability and/or proliferation includes an agent that dysregulates one or more mitotic kinase(s). In some embodiments, the one or more agent(s) that inhibit cancer cell viability and/or proliferation includes an agent that dysregulates one or more mitotic motor protein(s). In some embodiments, the one or more agent(s) that inhibit cancer cell viability and/or proliferation includes an agent that dysregulates an upstream regulator or a downstream effector of CPPC.

In some embodiments, the one or more agent(s) that inhibit cancer cell viability and/or proliferation includes a 4-phenoxy-quinoline derivative (e.g., LC30, LW33R, LXY18, LXY13, LC02 and LC09) or a 2-phenoxy-3,4′-bipyridine derivative (e.g., LG157). For example, in some embodiments, the one or more agent(s) that inhibit cancer cell viability and/or proliferation is a 4-phenoxy-quinoline derivative. In some embodiments, the one or more agent(s) that inhibit cancer cell viability and/or proliferation is a 2-phenoxy-3,4′-bipyridine derivative.

In some embodiments, the methods herein include administering one or more agent(s) that inhibit cancer cell viability and/or proliferation, wherein the one or more agent(s) that inhibit cancer cell viability and/or proliferation are any molecule described in International Publication number WO 2022/228549, incorporated herein by reference in its entirety. In some embodiments, the methods herein include administering one or more agent(s) that inhibit cancer cell viability and/or proliferation, wherein the one or more agent(s) that inhibit cancer cell viability and/or proliferation are any molecule described in International Publication number WO 2022/199654, incorporated herein by reference in its entirety.

### EXAMPLES

The disclosure is further illustrated by the following examples. The examples are provided for illustrative purposes only, and are not to be construed as limiting the scope or content of the disclosure in any way.

### Example 1: Methods and Compositions for the Diagnosis and Treatment of Cancer

Described herein is the development of a methodology for testing synthetic lethality in a large panel of cell lines that can be applied to compounds in subsequent stages of drug development. Also described are experiments which underly the surprising finding that an expression of MYC and MKLP2 that are elevated and decreased, respectively relative to a suitable control can serve as a diagnostic method in cancer.

### Materials and Methods

### Compounds

The compounds and respective concentrations tested included those described in Table 1 and Table 2, below.

TABLE 1
Compounds and Tested Concentration Ranges

| | | | | Tested |
|---|---|---|---|---|
| | | Expected | Reagent | concentration |
| Inhibitor | Target | IC₅₀ | Cat No. | range |
| Paprotrain | MKLP2 | IC50 = 1.35 μM, | TOCRIS, | About 10 nM |
| | | Ki = 3.36 μM | 4813-10 mg | to 20 μM |
| LC30 | — | — | — | About 2.5 nM |
| | | | | to 5 μM or about |
| | | | | 0.5 nM to 1 μM |
| LW33R | — | — | — | About 2.5 nM |
| | | | | to 5 μM |

TABLE 2
Compounds and Final Concentration Ranges

| Compound | Concentration Range (μM) | | | | | | | | | | | |
|---|---|---|---|---|---|---|---|---|---|---|---|---|
| Paprotrain | 20 | 10 | 5 | 2.5 | 1.25 | 0.6250 | 0.3125 | 0.1563 | 0.0781 | 0.0391 | 0.0195 | 0.0098 |
| LC30 | 1 | 0.5 | 0.25 | 0.125 | 0.0625 | 0.0313 | 0.0156 | 0.0078 | 0.0039 | 0.0020 | 0.0010 | 0.0005 |
| LW33R | 5 | 2.5 | 1.25 | 0.625 | 0.3125 | 0.1563 | 0.0781 | 0.0391 | 0.0195 | 0.0098 | 0.0049 | 0.0024 |

### Cell Culture

The human retinal pigment epithelial cell lines RPE-MYC^{H2B-GFP} and RPE-MYC^{Bc12} were cultured in DMEM (Gibco, Cat. No. 12100061) supplemented with 10% fetal bovine serum (Excell, Cat. No. FSP500 10099141), penicillin (100 U/mL)-streptomycin (100 μg/mL; Gibco, Cat. No. 15140-122), 2 mM L-glutamine (Gibco, Cat. No. 25030081), and 1 mM sodium pyruvate (Gibco, Cat. No. 11360070). Cell culture was maintained at 37° C. with 5% CO₂ in a humidified incubator. Mycoplasma contamination of cell lines was evaluated by using a Myco-Lumi™ Luminescent Mycoplasma Detection Kit (Beyotime, Cat. No. C0298M) before experiments were initiated. Each cell line was then expanded and stored in aliquots at −80° C. Each aliquot was used only for 10-15 passages, after which additional assays were conducted to examine contamination.

### Mechanism-Informed Phenotypic Screening Assay

A live cell, image-based screening assay was conducted to identify compounds that phenocopy the genetic loss of the chromosomal passenger protein complex. The screening cell line, RPE-MYC^{H2B-GFP}, expresses the chimeric protein histone 2B fused with a green fluorescent protein (H2B-GFP) to enable chromosome visualization. Screening cells were replated onto 96-well plates for 18-24 hours before exposure to test compounds, which were tested at concentrations between 10 nM and 20 μM with a 2-fold serial dilution. For any compound that was active at the lowest concentration of 10 nM, further dilution was conducted to determine the minimally effective concentration (MEC). At 24, 48, or 72 hours after initiation of treatment, cells were analyzed by a high-content fluorescent cell imager (GE IN-Cell Analyzer 2000). Cells treated with 0.1% DMSO were used as the vehicle control for all assays. To be scored as positive for a phenotypic change, a threshold value was chosen to reflect no less than a two-fold change elicited by the test compound. Values were considered accurate when the difference in MEC values between independent experiments were no more than two-fold. Four independent phenotypes were assayed: a. mitotic arrest; b. induction of polyploidy; c. cell death; and d. reduced cell number, described below.

a. Mitotic Arrest

Compounds were considered as inducing arrest if the percentage of mitotic cells at 24 hours was ≥10%. Mitotic cells were identified as rounded-up cells with condensed chromosomes with 4′,6-diamidino-2-phenylindole (DAPI) staining. The basal mitotic index (MI) of the screening cell line was 4.7%. An approximately two-fold increase of the MI was chosen to define the mitotic arrest.

b. Induction of Polyploidy:

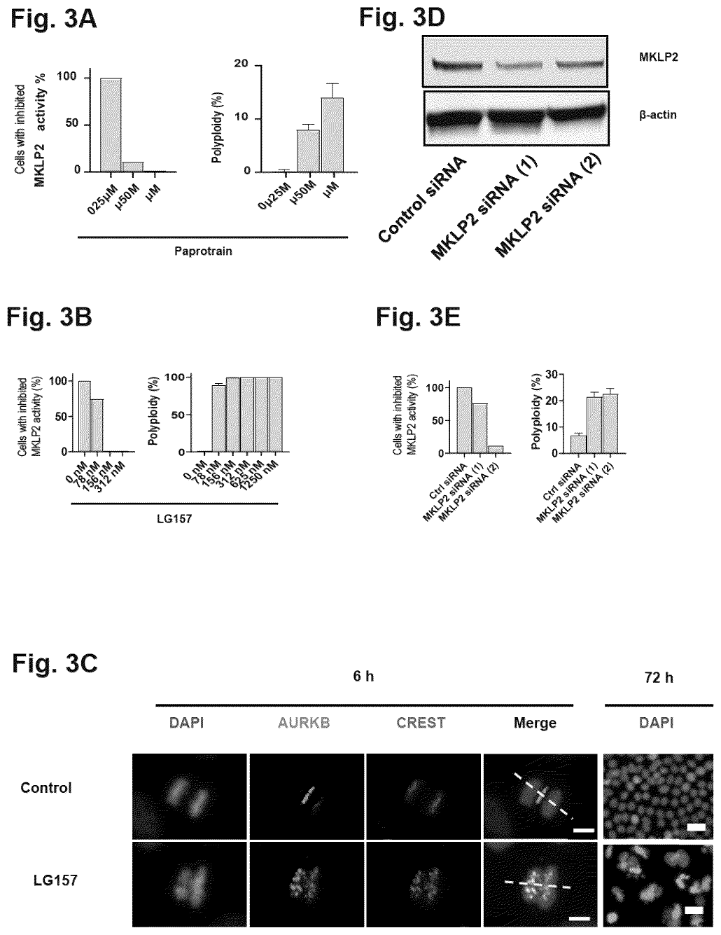

Compounds were considered positive for the induction of polyploidy when the percentage of polyploid cells in the interphase population, was ≥5% (with deduction of basal level of polyploid cells) at 72 hours after initiation of treatment (FIG. 3A right panel, FIG. 3B right panel, and FIG. 3E right panel). Polyploid cells were defined as those with multinucleated nuclei, visible upon DAPI staining. The basal level of spontaneous polyploid cells with the screening cell line was less than 1%.

c. Cell Death:

Compounds were considered positive for the induction of cell death when the presence of a substantial number of floating dead cells were found. The criterion for substantial cell death was determined when greater than or equal 1000 dead cells per well in a 96-well plate were observed, as determined by a follow-up trypan blue exclusion assay. The basal level of dead cells was less than 10 cells per well.

d. Reduced Cell Number

Compounds were considered positive for the ability to reduce cell number when the cell number at 72 hours was two-fold less than that of the vehicle control group.

Assay of Drug-Response with the MTT Assay

For passaging of cell lines prior to a 2,5-diphenyl-2H-tetrazolium bromide (MTT) assay, cells were routinely split when reaching mid-log phase and not allowed to progress to confluence. For the determination of cytotoxicity, cells were plated in 96-well plates at 4,000 cells per well in a volume of 100 μL and cultivated for 15 to 24 hours to reach 20-30% confluence. Cells on the plate were then exposed in triplicate to test compounds at concentrations ranging from 0.06 nM to 10 μM by using 3-fold serial dilutions or a concentration range from 5 nM to 10 μM using 2-fold serial dilutions. Cells exposed to vehicle (0.1% DMSO) were used as a negative control. Three days after initiation of the treatment, 20 μL of MTT (stock solution 5 mg/mL) was added to each well before incubation at 37° C. for another 3-4 hours. The medium was then aspirated with a microplate washer (BioTek ELX50) before 50 μL of DMSO was added to each well. After incubation with shaking for 10 minutes, the 96-well plate was analyzed with a microplate reader (BioTek ELX808iu) at a wavelength of 570 nm. The relative cell number was calculated as (A570^{Compound}/A570^{DMSO})×100%. Cell proliferation data were fit to a four parameter logistic curve with GraphPad Prism 8.0.2, which calculated the half-maximal inhibitory concentration (IC₅₀) with respect to the DMSO control wells, half-maximal effect concentration (EC₅₀), maximal activity area (AA), and E^{max} (100%−the percentage of the relative cell number at 10 Mm; FIGS. 22-23). Statistical analysis was performed in GraphPad Prism 8.0.2. Error bars were presented as mean±1 standard deviation.

### CellTiter-Glo® Luminescent Cell Viability Assay

The CellTiter-Glo® (CTG) assay determines levels of ATP, and therefore the metabolic health of cell grown in culture. The assay results correlate with the number of living cells over a large ATP concentration range. The assay was carried out according to the manufacturer's instructions (Promega, Cat. #G7570). Briefly, the CellTiter-Lumi™ Plus reagent (Beyotime, C0068M) was equilibrated to room temperature before use. The assay microplate and its contents were also equilibrated to room temperature for approximately 5-10 minutes. A volume of CellTiter-Lumi™ Plus reagent equal to the volume of cell culture medium in each well was added (e.g., add 100 μl of reagent to 100 μl of medium containing cells for a 96-well plate). Wells containing medium without cells were used as a baseline control to obtain a value for background luminescence. This was mixed for 5 minutes on an orbital shaker to induce cell lysis and then left at room temperature for 10 minutes to stabilize the luminescent signal. 150 μL of the mixture was transferred to a white 96-well microplate (Falcon, Cat. #353296) and the luminescence signal was recorded by a microplate reader (Tecan Spark).

### Data Normalization

The mean value of background luminescence was calculated and presented as Lum_{background}. The mean luminescence value of the vehicle control (0.1% DMSO) group was calculated and presented as Lum_{DMSO}. The cell viability in a drug treatment group was calculated as follows:

$$Cell viability = ( {Lum}_{compounds} - {Lum}_{backgroud} ) / ( {Lum}_{DMSO} - {Lum}_{backgroud} ) \times 100 \% .$$

If viability was more than 105% or less than 95% at low drug concentrations, all data were normalized again by setting the maximal viability to 100%. Concentration-response curves were rendered by GraphPad Prism 8.0.2. Descriptors of the curve including EC₅₀, IC₅₀, Slop, Eₘₐₓ, AUC, and AA were calculated using the program.

### Lactate Dehydrogenase Cytotoxicity Assay

The lactate dehydrogenase (LDH) assay—a plate-based colorimetric assay that determines cellular cytotoxicity—was performed. Released LDH in culture supernatants was measured at 30-minutes with a coupled enzymatic assay, which resulted in the conversion of a tetrazolium salt (iodonitrotetrazolium violet; INT) into a red formazan product. LDH was quantified as its enzymatic activity, which includes the oxidization of lactate to NADH, which reacts with INT in the presence of diaphorase to produce a red color. In this analysis, the amount of color formed is proportional to the number of lysed cells.

Briefly, to prepare for the LDH assay, buffer (Beyotime, C0017) was thawed prior to use, and LDH working reagent and LDH release reagent were prepared according the manufacturer's instructions. Wells containing medium without cells were used to determine a background optical density (OD) value. The maximum LDH release was calculated from wells where 10 μl of LDH release reagent was added to cells. At the end treatment, the plate was centrifuged at 2000 rpm for 3-5 minutes and 50 μl of the supernatant from wells was transferred to a fresh 96-well, flat-bottom (enzymatic assay) plate. 25 μl of LDH working reagent was added to each well and incubated for 15-30 minutes at room temperature with shaking in the dark. Variable incubation time resulted from variance in the time required for formation of color with different cell lines, indicative of variance in the level of LDH produced by cell lines. Absorbance was measured at 490 nm using a microplate reader (Tecan Spark), and dual wavelength measurements were performed using a wavelength of 600 nm as a reference wavelength.

### Data Normalization

The relative OD value was calculated as (the OD490 nm-the OD600 nm) for each well. A value for the mean background OD was also calculated (OD_{background}). This was subtracted from all values derived from the experimental wells. Values from wells given vehicle control (0.1% DMSO) were designated as OD_{DMSO}, while the mean OD value of the LDH Release group, given 10 μl of LDH release reagent, were calculated and designated as OD_{LDH max}. Values were used in the following formula to compute percent cytotoxicity:

$$Cell cytotoxicity = ( {OD}_{compounds} - {OD}_{DMSO} ) / ( {OD}_{LDH max} - {OD}_{DMSO} \times 100 \% .$$

If the value for cell cytotoxicity with a given compound was higher than 100%, the cytotoxicity was considered as more than 100%. If the value for cell cytotoxicity with a given compound was less than 0%, the cytotoxicity was considered 0%. Concentration-response curves were rendered by GraphPad Prism 8.0.2.

### Immunofluorescent Analysis AURKA/B Functionality

Immunofluorescent (IF) analysis was used to assess enzymatic activity (FIG. 3A left panel, FIG. 3B left panel, and FIG. 3E left panel) and localization of mitotic regulators. Cells were cultured prior to staining on 0.1% gelatin-coated glass coverslips in a six-well plate and allowed to adhere overnight at 37° C. in a humidified CO₂ incubator. In each experiment, four coverslips (14 mm diameter) were rested in each well of a 6-well plate and cells were allowed to adhere. After being exposed to a test compound for the time indicated in the respective figure legends, cells were washed with phosphate buffered saline (PBS), pH 7.4, 3 times, before being fixed for 10 minutes with 4% paraformaldehyde (PFA) in PBS, containing 0.5% Triton X-100 for membrane permeabilization. The cells were then blocked with 5% bovine serum albumin (BSA) for 1 hour at room temperature before the initiation of IF staining. Staining was typically performed by sequential incubation of coverslips with a primary antibody for 2 hours at room temperature or overnight at 4° C., followed by fluorophore-labelled secondary antibody for 1 hour at room temperature before visualization.

The primary antibodies used for IF quantification of kinase activity were those included in the surrogate marker method for AURKA and AURKB. Such antibodies included a rabbit AURKA Thr288P antibody (Cat. No. 3079 Cell Signaling Technology) and a rabbit H3Ser10P antibody (Cat. No. 53348 Cell Signaling Technology). To quantify kinase activity for AURKA and AURKB, the immunofluorescent intensity of the AURKA Thr288P and H3Ser10P signals were quantified with ImageJ software. Ten randomly chosen cells, all at a similar stage of mitosis, were quantified and data were normalized to the mean intensity obtained with mitotic cells in the vehicle control group (0.1% DMSO).

To examine the mitotic localization of AURKB, a rabbit AURKB antibody (Cat. No. abs131460; Absin; Shanghai, China) was used (FIG. 3C). Human autoimmune serum from a patient with CREST syndrome, which recognizes a variety of kinctochore proteins, was a gift from B. R. Brinkley (Baylor College of Medicine, Houston). For assessing the localization of other mitotic regulators, a mouse PLK1 antibody (ZYMED, Cat. No. 33-1700 Santa Cruz Biotechnology) and a rabbit MKLP1 antibody (Cat. No. sc-867 Santa Cruz Biotechnology) were utilized. A mouse anti-β-tubulin antibody (Cat. No. T5201 Sigma-Aldrich) was also used. Secondary antibodies included Rhodamine Red™-X-conjugated AffiniPure Donkey Anti-Human IgG (H+L) (Cat. No. 709-295-149 Jackson ImmunoResearch), Fluorescein (FITC)-conjugated Affinipure Goat Anti-Mouse IgG (H+L) (Cat. No. 115-095-003 Jackson ImmunoResearch) and Rhodamine (TRITC) AffiniPure Goat Anti-Rabbit IgG (H+L) (Cat. #111-025-003 Jackson ImmunoResearch). After being mounted in a DAPI-containing Fluoromount-GTM medium (36308ES20, YEASEN), cells were subjected to image acquisition and processing using an EVOS FL Auto Cell Imaging System (Thermo-Fisher).

Selective Knockdown of MKLP2 with siRNA

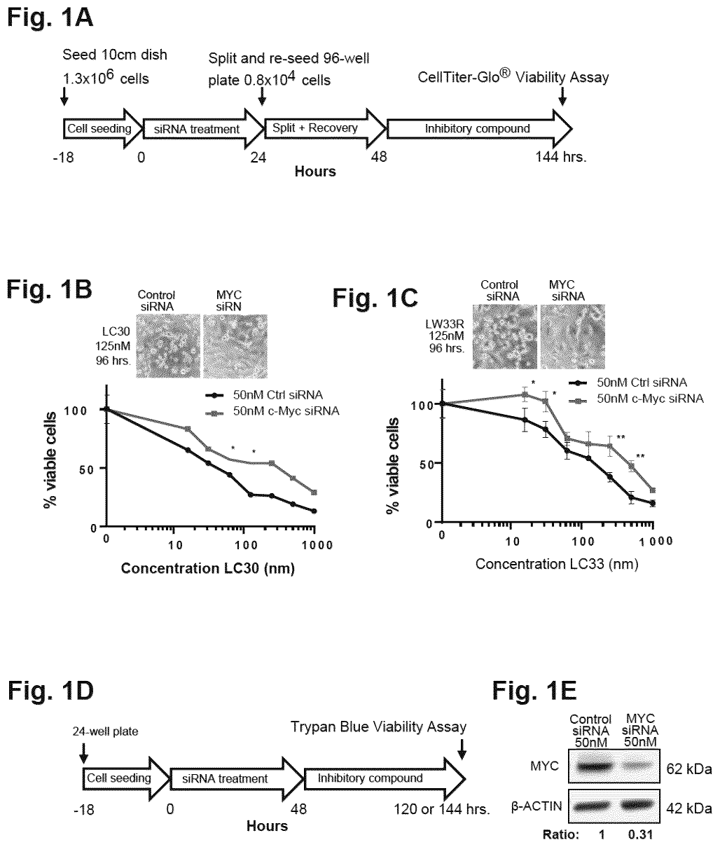

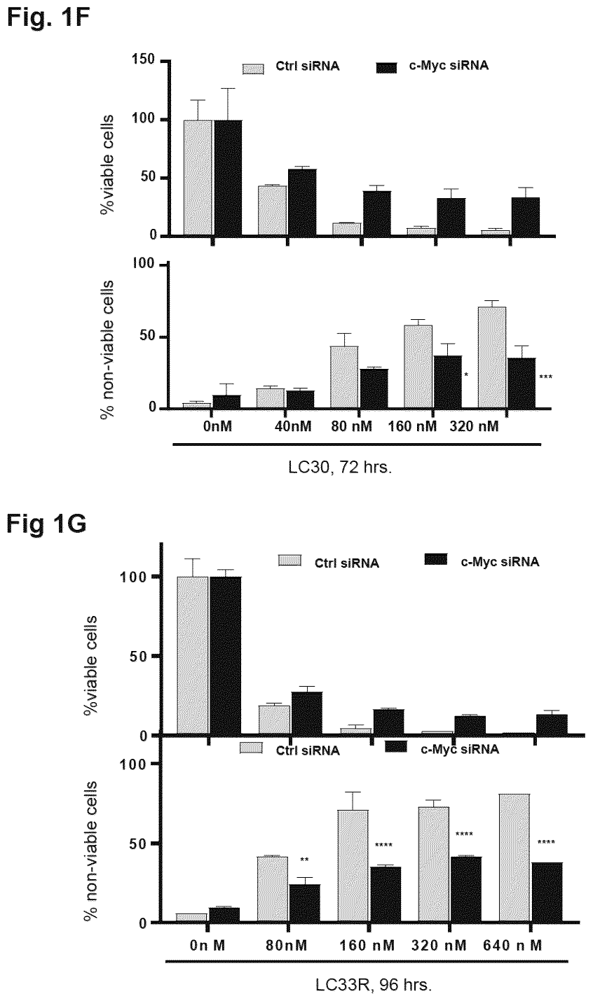

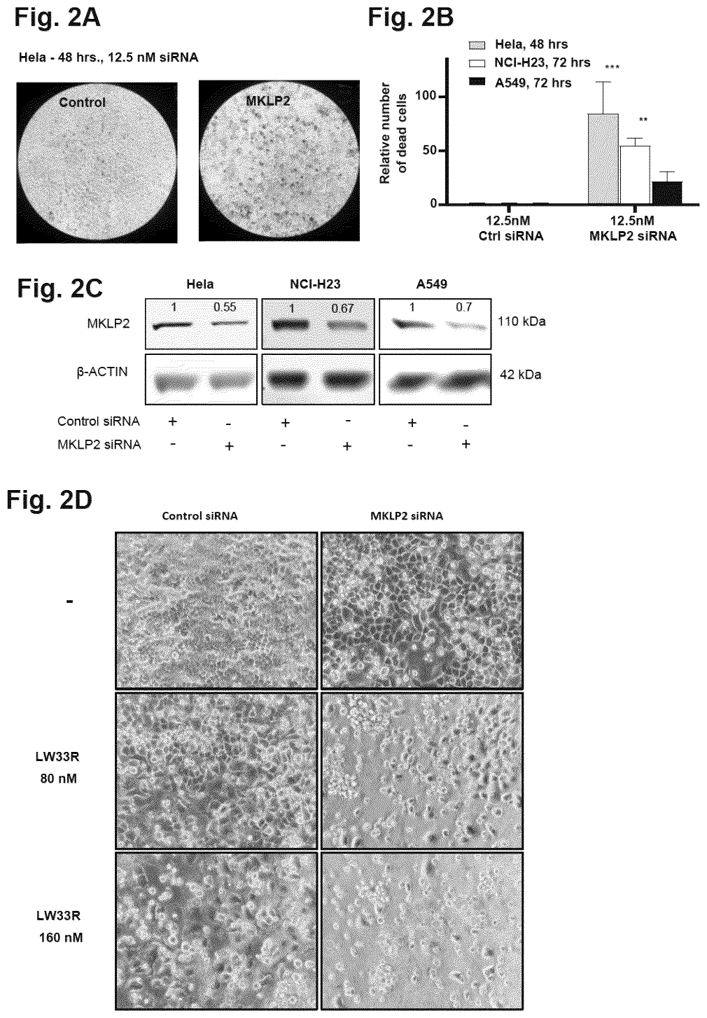

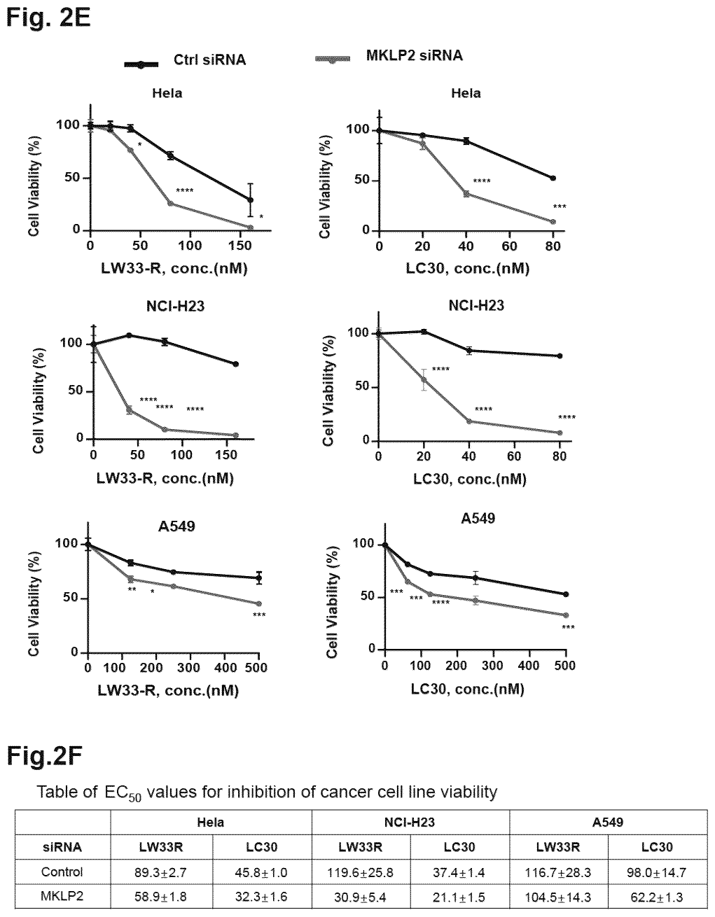

Cells were passed onto 24-well plates at a density of 4×10⁴ cells/well, eighteen hours prior to transfection. Transfection was with 12.5 nM, 25 nM, or 50 nM of MKLP2 siRNA or with a negative control siRNA, respectively. Two or three days after initiation of siRNA transfection, a portion of cells were collected and analyzed for viability by the trypan blue exclusion assay (FIG. 1D, FIG. 2A, FIG. 2D), while another portion of cells were collected to analyze the degree of knockdown through expression of MKLP2 and β-Actin protein using immunoblot analysis (FIG. 1E). The remaining cells were exposed to compounds, such as LW33R and LC30, at different concentrations (FIGS. 1F-1G). After exposure of the siRNA-transfected cells for 3 days, cells were collected to analyze viability by the CellTiter-Glo® luminescent cell viability assay (FIG. 1A, CTG assay, Promega, Cat. #G7570) to generate concentration-response curves and comparative metrics such as the half-maximal effective concentration (EC₅₀) value (FIG. 2F). Each independent experiment was conducted in duplicate.

The MKLP2 and control siRNAs (GenePharma) were transfected into cells with lipofectamine 2000 (ThermoFisher, Cat. #11668027), according to the manufacturer's instructions and knockdown was confirmed (FIGS. 1B-1C, FIGS. 2B-2C, FIG. 2E, and FIG. 3D). The siRNAs, as provided in Table 3, below, were as follow:

TABLE 3
siRNAs for MKLP2 knockdown
MKLP2 siRNA 1: MKLP2-homo-1993 siRNA (5′ to 3′)

| | |
|---|---|
| Sense sequence | GUUCUCAGCCAUUGCUAGCTT |
| Antisense sequence | GCUAGCAAUGGCUGAGAACTT |

MKLP2 siRNA 2: MKLP2-homo-2823 siRNA (5′ to 3′)

| | |
|---|---|
| Sense sequence | CCACUUGUGAUGACAUCUUTT |
| Antisense sequence | AAGAUGUCAUCACAAGUGGTT |

Control siRNA (5′ to 3′)

| | |
|---|---|
| Sense sequence | UUCUCCGAACGUGUCACGUTT |
| Antisense sequence | ACGUGACACGUUCGGAGAATT |

siRNA-Mediated Depletion of MYC

Two different procedures were utilized to deplete MYC in cells, with the protocols varying in the timing of siRNA treatment as outlined, immediately below.

a. Protocol One

Cells were passed onto a 10 cm culture plate at an initial density of 1.3×10⁶ cells. Eighteen hours after seeding, cells were transfected with 50 nM of MYC siRNA or control siRNA, respectively, for 24 hours. Transfected cells were then passed onto 96-well plates at a density of 8,000 cells per well. Small molecule inhibitors with antimitotic activity were then added at varying concentrations. Cell viability was assayed by the CTG assay, 3-4 days after initiation of the drug treatment.

b. Protocol Two

Cancer cells were seeded in a 24-well plate at a density of 4×10⁴ cells per well and equilibrated for 18 hours before transfection with 50 nM of either a control or MYC siRNA. After 48 hours, the transfected cells were exposed for an additional 3-4 days to a small molecule inhibitor at the concentrations indicated. At the end of treatment, cell viability was assessed by the trypan blue exclusion assay. To assess the degree of knockdown, MYC and β-ACTIN were measured by immunoblot analysis three days after the start of siRNA transfection.

### Immunoblot Analysis

RIPA buffer (50 mM Tris, 150 mM NaCl, 1% TritonX-100, 1% sodium deoxycholate, and 0.1% SDS) was used for cell lysis and was supplemented with a cocktail of phosphatase (Beyotime, #P1082) and protease inhibitors (Beyotime, #P1005). Lysates were collected and placed on ice for 15 minutes before sonication for 10 seconds. Supernatants were cleared by centrifugation at 12000 rpm for 15 minutes. The total protein concentration was quantified with a Bicinchoninic acid assay (BCA) kit (Sangon Biotech, Cat. No. C503051-0500) according to manufacturer's instructions. 20-80 μg of protein was loaded into each well of a precast polyacrylamide gel (Invitrogen, NP0336BOX or NP0321BOX) and proteins were separated under a voltage of 100-130V for 1-2 hours. The separated proteins were transferred to a 0.22 μm PVDF membrane for 80 minutes under a constant voltage of 70 V. The membrane was blocked for 1.5 h with 5% nonfat milk (Sangon, A600669-0250) in a TBST buffer at room temperature before being incubated overnight at 4° C. with a primary antibody in Odyssey® blocking buffer (Li-COR, Inc., Lincoln, NE) containing 0.2% Tween 20 (or in PBS containing 0.2% Tween 20). The next day, the membrane was washed and incubated with a secondary antibody at room temperature for 1 hour. The primary antibodies used were a rabbit MYC monoclonal (clone Y69, 1:1000 dilution; Abcam #GR3272831-14), a rabbit MYCN (Aab24193, 1:1000 dilution; Abcam), a rabbit MYCL (76266S, 1:1000 dilution; Cell Signaling Technology), a mouse monoclonal MKLP2/MKLP2 antibody (D-3, Cat. #sc-374508, 1:100 dilution; Santa Cruz Biotechnology), and a mouse β-ACTIN antibody (Cat. #. 66009-1, 1:1000 dilution; Proteintech). Secondary antibodies used were IRDye® 800CW Goat anti-Mouse IgG (Cat. #. 926-32210, Lot #C91210-09) used at a 1:10000 dilution or IRDye® 680RD Goat anti-Rabbit antibody (926-68071, Lot #D00115-06) used at a 1:5000 dilution. Images were obtained from using an Odyssey CLx Imaging System and processed with Image Studio Ver 5.2.

Development of an expression-based comparative metrics for MYC and MKLP

To develop a comparative metric reflecting the level of MYC and/or MKLP2 in a given cell line, the signal intensity of the MYC and MKLP2 protein bands from immunoblot analysis were normalized to the intensity of the housekeeping protein β-ACTIN. The baseline was set as 1: equal to the intensity of MYC or MKLP2 in the model cell line RPE-MYCH2BGFP, which is engineered to ectopically overexpress MYC. Whole cell MYC and MKLP2 proteins were quantified in a panel of 98 human cancer cell lines, see Table 4, below. Each cell line provides a data point for the MYC and MKLP2 expression level.

TABLE 4
Panel of Cancer Cell Lines

| Code | Tissue | Cell line |
|---|---|---|
| 1 | Blood | Raji-Luc |
| 2 | Blood | Daudi |
| 3 | Blood | RAMOS |
| 4 | Blood | U-937 |
| 5 | Blood | HL-60 |
| 6 | Blood | K-562 |
| 7 | Blood | Jurkat |
| 8 | Blood | HPB-ALL |
| 9 | Blood | MOLT-4 |
| 10 | Blood | CCRF-CEM |
| 11 | Blood | IM-9 |
| 12 | Blood | RPMI 8226 |
| 13 | Blood | NCI-BL1672 |
| 14 | Lung | NCI-H146 |
| 15 | Lung | NCI-H128 |
| 16 | Lung | NCI-H209 |
| 17 | Lung | NCI-H2171 |
| 18 | Lung | NCI-H211 |
| 19 | Lung | NCI-H510A |
| 20 | Lung | NCI-H524 |
| 21 | Lung | NCI-H526 |
| 22 | Lung | NCI-H82 |
| 23 | Lung | NCI-H841 |
| 24 | Lung | NCI-H187 |
| 25 | Lung | NCI-H446 |
| 26 | Lung | NCI-H1299 |
| 27 | Lung | NCI-H460 |
| 28 | Lung | NCI-H23 |
| 29 | Lung | NCI-H596 |
| 30 | Lung | NCI-H2170 |
| 31 | Lung | NCI-H520 |
| 32 | Lung | A549 |
| 33 | Lung | Calu6 |
| 34 | Lung | HOP62 |
| 35 | Lung | A427 |
| 36 | Breast | BT-549 |
| 37 | Breast | BT-474 |
| 38 | Breast | MDA-MB-157 |
| 39 | Breast | T47D |
| 40 | Breast | MCF7 |
| 41 | Breast | MDA-MB-231 |
| 42 | Breast | MDA-MB-415 |
| 43 | Breast | MDA-MB-436 |
| 44 | Breast | MDA-MB-468 |
| 45 | Breast | MDA-MB-361 |
| 46 | Breast | BT-20 |
| 47 | Breast | HCC1569 |
| 48 | Breast | MDA-MB-435 |
| 49 | Breast | ZR-75-1 |
| 50 | Breast | PL-3 |
| 51 | Breast | MDA-MB-461 |
| 52 | Breast | MDA-MB175VII |
| 53 | Breast | SW613 |
| 54 | Kidney | ACHN |
| 55 | Kidney | TUWI |
| 56 | Kidney | SK-N-EP1 |
| 57 | Stomach | SNU-5 |
| 58 | Stomach | HTB-135 |
| 59 | Stomach | NCI-N87 |
| 60 | Prostate | DU-145 |
| 61 | Prostate | LNCaP |
| 62 | Prostate | VCaP |
| 63 | Ovary | SKOV3 |
| 64 | Ovary | OVCAR3 |
| 65 | Ovary | Caov3 |
| 66 | Ovary | SW626 |
| 67 | Ovary | PA-1 |
| 68 | Ovary | NCI-ADR-RES |
| 69 | Intestine | HT-29 |
| 70 | Intestine | HCT-116 |
| 71 | Intestine | SW480 |
| 72 | Intestine | HCT-15 |
| 73 | Intestine | SW680 |
| 74 | Bone | Saos-2 |
| 75 | Bone | U2OS |
| 76 | Bone | HOS |
| 77 | Cervix | Hela |
| 78 | Cervix | C-33 A |
| 79 | Skin | A431 |
| 80 | Skin | UACC-257 |
| 81 | Skin | SK-MEL-5 |
| 82 | Skin | UACC-62 |
| 83 | Skin | SK-MEL-28 |
| 84 | Skin | C32 |
| 85 | Liver | SK-Hep1 |
| 86 | Liver | HepG2 |
| 87 | Pancreas | BxPC-3 |
| 88 | Pancreas | MIA Paca-2 |
| 89 | Pancreas | PANC-1 |
| 90 | Pancreas | AsPC-1-Luc |
| 91 | Brain | AG2203 |
| 92 | Brain | SK-N-SH |
| 93 | Brain | SK-N-AS |
| 94 | Brain | SK-N-BE2 |
| 95 | Brain | IMR-32 |
| 96 | Brain | T98G |
| 97 | Brain | U251 |
| 98 | Brain | Kelly |

The number of cell lines were equal to the number of data points for a given analysis, such that up to 98 data points were used to determine the most discriminatory cut-off of high- and low-responsivity groups for MYC and MKLP2. To ensure each group had sufficient observations, cutoff points within the middle 70% of values were considered for bifurcation only. Therefore, if 95 cell lines were assayed for a given drug, 65 out of 95 were potential cut-offs for either MYC or MKLP2. When considering both proteins simultaneously, the number of combined, MYC and MKLP2 cut-off points, were 652 (4225 cut-off points in total). For each of the 4225 combined cutoff points, the optimal threshold for concentration-response metrics were determined, which was accomplished using cutpointr (version 1.1.2), an R package for tidy calculation of optimal cutpoints (version 4.1.2).

For each combined cut-off, the p-value for three tests were calculated, including the Fisher exact test, the Barnard test, and the G-test. Cut-off points were selected when the p-values of all three tests were less than or equal to 0.05. Selected thresholds were further filtered to have the sum of sensitivity and specificity greater than 1.3 and the number of true positives equal to or greater than 25.

### Additional Comparators

To quantify the entire MYC protein family, MYC, MYCN, and MYCL were measured in a panel of 98 human cancer cell lines of various origins. Such groups were then divided into the MYC high group and MYC low group based on the abundance of all three MYC family members. The same set of conclusions were reached when cells with only abundant MYCN or MYCL were counted, as in the MYC high group. Preferential killing of cell lines considered to fall into the MYC high cell group were also evident when cell lines from specific tissues or indications were considered separate from other cell lines. For example, lung cancer cell lines were analyzed separately and the relationship between MYC, MKLP2, and drug response was found to exist.

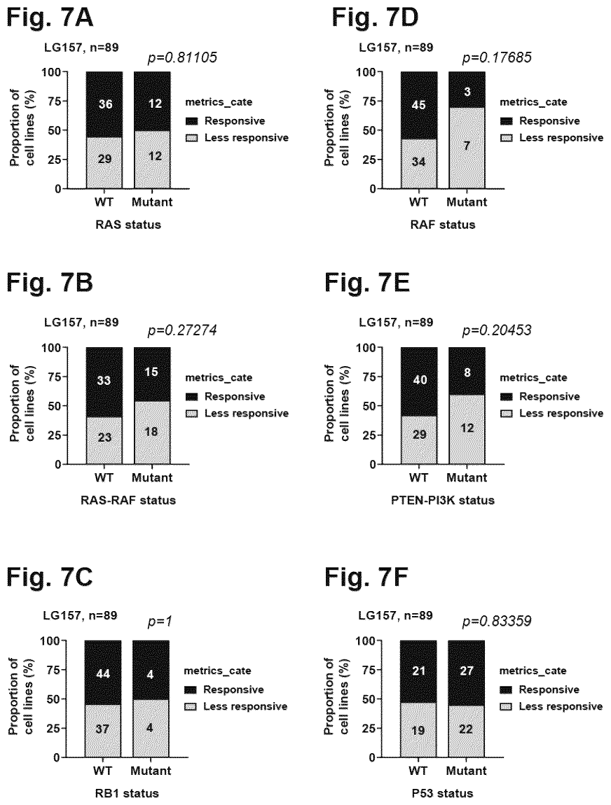

A similar correlation analysis was performed between drug response and other oncogenic alterations, such as inactivation of the tumor suppressors P53, RB1, and PTEN, as well as activation of oncogenes such as RAS, BRAF, and PI3K in the 98 cell line panel (FIGS. 7A-7F). An additional comparator included RAF. No correlation was observed. Taken together this finding reveals the utility of MYC and MKLP2 expression as predictors-of-response to drugs that disrupt the localization of mitotic regulators.

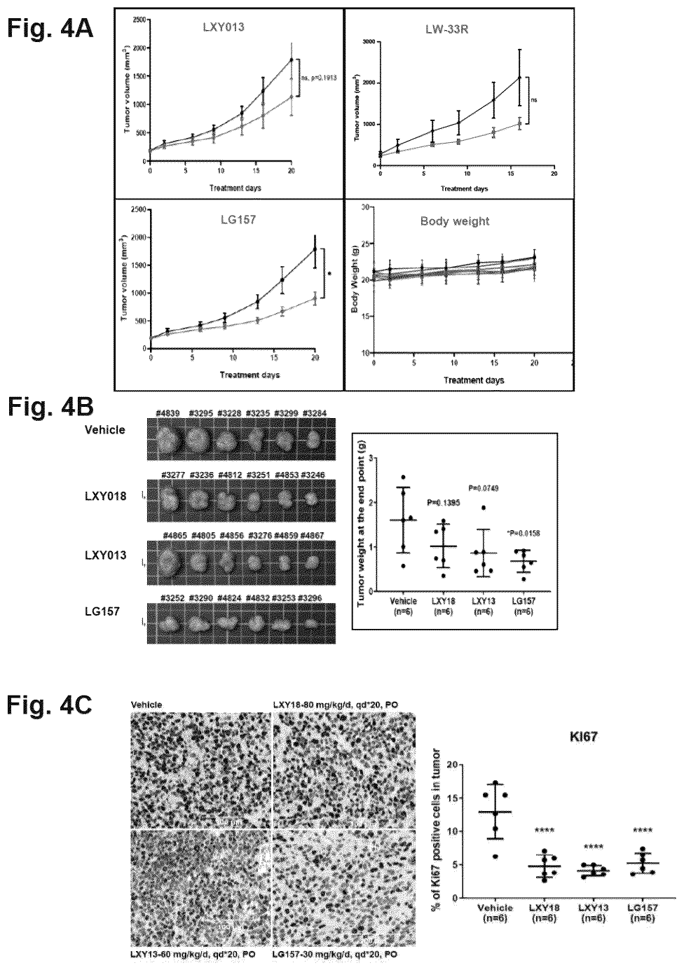

Additionally, the volume and weight of tumors in response to the compounds described herein were assessed (FIG. 4A-4B) as well as immunohistochemistry analyses and quantification of Ki-67 expression in xenografts (FIG. 4C).

### Dose-Response Metrics

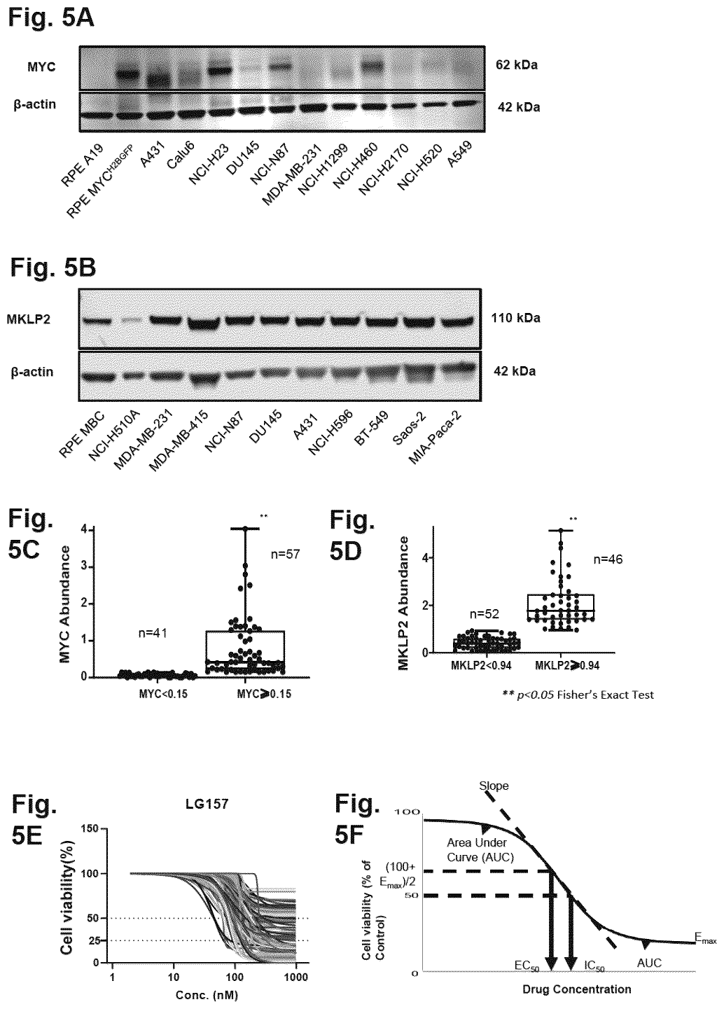

For each compound assayed in the up to 98 different cell lines, multiple comparative metrics were calculated from the best-fit concentration-response curve. The following concentration-response data were calculated from experimental MTT or CTG data and collected across the entire cell line panel (up to 98 cell lines) for each compound:
1. IC₅₀—concentration at which the relative response was 50% compared to vehicle control (0.1% DMSO);
2. Eₘₐₓ—maximal effect of the drug compared to vehicle control;
3. AUC—the area under the curve, which is the area below the fitted dose-response curve;
4. EC₅₀—concentration at half-maximal effect relative to vehicle control (FIG. 5F); and
5. h—the Hill coefficient of the fitted dose-response curve.

Dose-response metrics were calculated using a four parameter logistic model by the GRmetrics package (version 1.2.0) in R (version 4.1.2).

### Statistical Methods

While sensitivity measures the test's ability to correctly detect responders, specificity measures the test's ability to reject non-responders. For the analyses described here, the following rules were applied:

$$Sensitivity = True Positives / ( True Positives + False Negatives ) ; and$$

$$Specificity = True Negatives / ( True Negatives + False Positives )$$

A metric was considered as the optimized the sum of sensitivity and specificity in order to determine the best division of cell line data into highly responding cell lines versus low responding cell lines. Specifically, (y=sensitivity+specificity), where y was optimized for the data, across the up to 98 cell line test panel, see Table 5, below. Statistical tests were used to demonstrate that the difference in groups was not attributed to random chance alone, including the below-described tests. Results of the statistical analyses are provided in Tables 6-8, below.

TABLE 5
Cancer Cell Lines Used and Respective Responsivity

| Cell | | LC30 | LC30 | LW33R | | | CMYC_{—} | MKLP2_{—} | LW33R | LW33R |
|---|---|---|---|---|---|---|---|---|---|---|
| Line | LC30 AUC | AUC_cate | Rank | AUC | CMYC | MKLP2 | cate | cate | AUC_cate | Rank |
| NCI- | 0.55121987 | Responsive | 2 | 0.53025108 | 0.35 | 0.1 | High | Low | Responsive | 5 |
| H520 | | | | | | | | | | |
| HeLa | 0.72919378 | Responsive | 18 | 0.60393056 | 4.04 | 1.57 | High | Low | Responsive | 15 |
| CALU6 | 0.76559797 | Responsive | 29 | 0.59705517 | 0.48 | 0.8 | High | Low | Responsive | 13 |
| HOP62 | 0.90328753 | Less | 62 | 0.89220258 | 0.03 | 2.3 | Low | High | Less | 58 |
| | | responsive | | | | | | | responsive | |
| MDA | 0.90681259 | Less | 65 | 0.84552322 | 0.03 | 2.8 | Low | High | Less | 54 |
| MB231 | | responsive | | | | | | | responsive | |
| Aspc- | — | Less | — | — | 0.07 | 0.8 | Low | Low | Less | — |
| 1-Luc | | responsive | | | | | | | responsive | |

### Fisher's Exact Test

Fisher's exact test was used when determining if the association between two categorical variables (e.g., MYC protein expression level and response) was non-random. The null distribution for computing p values was the hypergeometric distribution. The hypergeometric distribution is a discrete probability distribution that describes the probability of k success in n draws, without replacement, in a finite sample. P values less than 0.05 indicated a significant statistical association between the two categorical variables and implied dependence between the two variables.

Fisher's exact test p values were calculated with the fisher.test function of the stats package in R version 4.1.2.

### Barnard Test

Barnard's test is a non-parametric alternative to Fisher's exact test which can be used, for example, for 2×2 tables. The Barnard test was used for 2×2 comparison with the MYC and MKLP2 comparators. Bernard's exact test p values were calculated with the R function for Barnard's exact test in the stats package in R version 4.1.2.

### G-Test

The G-test of independence is a likelihood ratio test that tests the goodness of fit of observed frequencies to their expected frequencies if row and column classifications are independent. The method is based on the multinomial distribution where both row and column totals are random, not fixed.

TABLE 6
Statistical Analysis of MYC and MKLP2 Expression as a Predictive
Biomarker Combination for LG157 Area Under the Curve

| | | | | | | | | | Sum of |
|---|---|---|---|---|---|---|---|---|---|
| MYC | MKLP2 | Metric | | | | | | | sensitivity |
| cutoff | cutoff | cutoff | | | Fisher | Barnard | | # of true | and |
| value | value | value | Sensitivity | Specificity | p-value | p-value | G-test | positives | specificity |
| 0.06 | 2.14 | 0.73450 | 0.66129 | 0.66667 | 0.00298 | 0.00212 | 0.00157 | 41 | 1.32796 |
| 0.06 | 2.01 | 0.73450 | 0.65574 | 0.64865 | 0.00619 | 0.00398 | 0.00321 | 40 | 1.30439 |
| 0.10 | 2.14 | 0.73450 | 0.68421 | 0.65854 | 0.00102 | 0.00086 | 0.00071 | 39 | 1.34275 |
| 0.09 | 2.14 | 0.73450 | 0.67241 | 0.65000 | 0.00206 | 0.00192 | 0.00153 | 39 | 1.32241 |
| 0.08 | 2.14 | 0.73450 | 0.66102 | 0.64103 | 0.00402 | 0.00368 | 0.00315 | 39 | 1.30204 |
| 0.10 | 2.01 | 0.73450 | 0.67857 | 0.64286 | 0.00211 | 0.00169 | 0.00146 | 38 | 1.32143 |
| 0.09 | 2.01 | 0.73450 | 0.66667 | 0.63415 | 0.00412 | 0.00355 | 0.00303 | 38 | 1.30081 |
| 0.06 | 1.36 | 0.75256 | 0.78723 | 0.58824 | 0.00020 | 0.00014 | 0.00012 | 37 | 1.37547 |
| 0.04 | 1.27 | 0.75256 | 0.78723 | 0.58824 | 0.00020 | 0.00014 | 0.00012 | 37 | 1.37547 |
| 0.05 | 1.36 | 0.75256 | 0.77083 | 0.58000 | 0.00049 | 0.00043 | 0.00034 | 37 | 1.35083 |
| 0.06 | 1.37 | 0.75256 | 0.77083 | 0.58000 | 0.00049 | 0.00043 | 0.00034 | 37 | 1.35083 |
| 0.04 | 1.31 | 0.75256 | 0.77083 | 0.58000 | 0.00049 | 0.00043 | 0.00034 | 37 | 1.35083 |
| 0.06 | 1.78 | 0.73222 | 0.64912 | 0.68293 | 0.00196 | 0.00124 | 0.00106 | 37 | 1.33205 |
| 0.06 | 1.90 | 0.73222 | 0.63793 | 0.67500 | 0.00377 | 0.00242 | 0.00213 | 37 | 1.31293 |
| 0.10 | 2.00 | 0.73450 | 0.67273 | 0.62791 | 0.00420 | 0.00344 | 0.00286 | 37 | 1.30063 |
| 0.15 | 2.14 | 0.73440 | 0.76596 | 0.68627 | 0.00001 | 0.00001 | 0.00000 | 36 | 1.45223 |
| 0.14 | 2.14 | 0.73331 | 0.73469 | 0.71429 | 0.00002 | 0.00001 | 0.00001 | 36 | 1.44898 |
| 0.14 | 2.01 | 0.73440 | 0.75000 | 0.68000 | 0.00002 | 0.00002 | 0.00001 | 36 | 1.43000 |
| 0.06 | 1.27 | 0.75256 | 0.81818 | 0.59259 | 0.00004 | 0.00003 | 0.00002 | 36 | 1.41077 |
| 0.13 | 2.14 | 0.73222 | 0.69231 | 0.69565 | 0.00023 | 0.00016 | 0.00010 | 36 | 1.38796 |
| 0.06 | 1.31 | 0.75256 | 0.80000 | 0.58491 | 0.00017 | 0.00010 | 0.00008 | 36 | 1.38491 |
| 0.05 | 1.27 | 0.75256 | 0.80000 | 0.58491 | 0.00017 | 0.00010 | 0.00008 | 36 | 1.38491 |
| 0.12 | 2.14 | 0.73222 | 0.67925 | 0.68889 | 0.00050 | 0.00036 | 0.00024 | 36 | 1.36813 |
| 0.04 | 1.24 | 0.75256 | 0.78261 | 0.57692 | 0.00042 | 0.00026 | 0.00024 | 36 | 1.35953 |
| 0.05 | 1.31 | 0.75256 | 0.78261 | 0.57692 | 0.00042 | 0.00026 | 0.00024 | 36 | 1.35953 |
| 0.06 | 1.34 | 0.75256 | 0.78261 | 0.57692 | 0.00042 | 0.00026 | 0.00024 | 36 | 1.35953 |
| 0.05 | 1.34 | 0.75256 | 0.76596 | 0.56863 | 0.00098 | 0.00077 | 0.00064 | 36 | 1.33458 |
| 0.11 | 2.14 | 0.73222 | 0.65455 | 0.67442 | 0.00209 | 0.00135 | 0.00110 | 36 | 1.32896 |
| 0.06 | 1.62 | 0.73222 | 0.65455 | 0.67442 | 0.00209 | 0.00135 | 0.00110 | 36 | 1.32896 |
| 0.06 | 1.77 | 0.73222 | 0.64286 | 0.66667 | 0.00402 | 0.00259 | 0.00223 | 36 | 1.30952 |
| 0.06 | 1.68 | 0.73222 | 0.64286 | 0.66667 | 0.00402 | 0.00259 | 0.00223 | 36 | 1.30952 |
| 0.06 | 1.76 | 0.73222 | 0.64286 | 0.66667 | 0.00402 | 0.00259 | 0.00223 | 36 | 1.30952 |
| 0.14 | 1.78 | 0.73440 | 0.77778 | 0.67925 | 0.00001 | 0.00000 | 0.00000 | 35 | 1.45702 |
| 0.15 | 2.01 | 0.73440 | 0.76087 | 0.67308 | 0.00002 | 0.00001 | 0.00001 | 35 | 1.43395 |
| 0.14 | 1.90 | 0.73440 | 0.76087 | 0.67308 | 0.00002 | 0.00001 | 0.00001 | 35 | 1.43395 |
| 0.14 | 2.00 | 0.73440 | 0.74468 | 0.66667 | 0.00005 | 0.00004 | 0.00003 | 35 | 1.41135 |
| 0.06 | 1.24 | 0.75256 | 0.81395 | 0.58182 | 0.00008 | 0.00006 | 0.00005 | 35 | 1.39577 |
| 0.13 | 1.78 | 0.73440 | 0.72917 | 0.66000 | 0.00013 | 0.00010 | 0.00009 | 35 | 1.38917 |
| 0.08 | 1.36 | 0.75256 | 0.79545 | 0.57407 | 0.00038 | 0.00019 | 0.00016 | 35 | 1.36953 |
| 0.05 | 1.24 | 0.75256 | 0.79545 | 0.57407 | 0.00038 | 0.00019 | 0.00016 | 35 | 1.36953 |
| 0.13 | 2.01 | 0.73222 | 0.68627 | 0.68085 | 0.00052 | 0.00033 | 0.00024 | 35 | 1.36713 |
| 0.12 | 2.01 | 0.73222 | 0.67308 | 0.67391 | 0.00109 | 0.00077 | 0.00053 | 35 | 1.34699 |
| 0.10 | 1.78 | 0.73222 | 0.67308 | 0.67391 | 0.00109 | 0.00077 | 0.00053 | 35 | 1.34699 |
| 0.07 | 1.36 | 0.75256 | 0.77778 | 0.56604 | 0.00087 | 0.00048 | 0.00045 | 35 | 1.34382 |
| 0.08 | 1.37 | 0.75256 | 0.77778 | 0.56604 | 0.00087 | 0.00048 | 0.00045 | 35 | 1.34382 |
| 0.10 | 1.90 | 0.73222 | 0.66038 | 0.66667 | 0.00219 | 0.00160 | 0.00113 | 35 | 1.32704 |
| 0.09 | 1.78 | 0.73222 | 0.66038 | 0.66667 | 0.00219 | 0.00160 | 0.00113 | 35 | 1.32704 |
| 0.08 | 1.40 | 0.75256 | 0.76087 | 0.55769 | 0.00191 | 0.00138 | 0.00117 | 35 | 1.31856 |
| 0.07 | 1.37 | 0.75256 | 0.76087 | 0.55769 | 0.00191 | 0.00138 | 0.00117 | 35 | 1.31856 |
| 0.11 | 2.01 | 0.73222 | 0.64815 | 0.65909 | 0.00422 | 0.00297 | 0.00229 | 35 | 1.30724 |

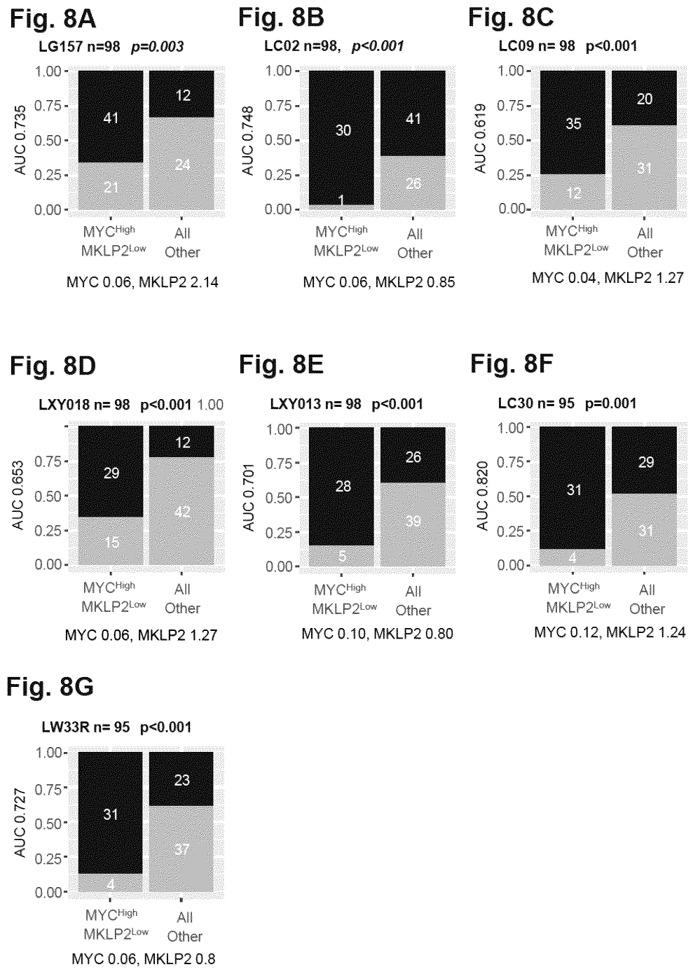

In this table the top 50 out of 388 statistically significant combinations of MYC and MKLP2 cutoff values were shown. Data is sorted to maximize the number of true positives in the analysis. Row 1 statistical values apply to data in FIG. 8A. Sensitivity was measured as true positives/(true positives+false negatives). Specificity was measured as true negatives/(true negatives+false positives). The number of true positives were the (number of responsive cell lines in the MYC high and MKLP2 low group)/total number of cell lines.

TABLE 7
Statistical analysis of MYC and MKLP2 expression as
a predictive biomarker combination for LG157 EC50

| | | | | | | | | | Sum of |
|---|---|---|---|---|---|---|---|---|---|
| MYC | MKLP2 | Metric | | | | | | Number | Sensitivity |
| Cutoff | Cutoff | Cutoff | | | Fisher | Barnard | | of True | and |
| Value | Value | Value | Sensitivity | Specificity | p-value | p-value | Gtest | Positives | Specificity |
| 0.33 | 2.01 | 102.29283 | 0.80645 | 0.55224 | 0.00098 | 0.00056 | 0.00060 | 25 | 1.35869 |
| 0.33 | 2.14 | 102.29283 | 0.80645 | 0.55224 | 0.00098 | 0.00056 | 0.00060 | 25 | 1.35869 |
| 0.32 | 2.01 | 102.29283 | 0.78125 | 0.54545 | 0.00248 | 0.00171 | 0.00173 | 25 | 1.32670 |
| 0.32 | 2.14 | 102.29283 | 0.78125 | 0.54545 | 0.00248 | 0.00171 | 0.00173 | 25 | 1.32670 |
| 0.16 | 2.00 | 88.71336 | 0.63415 | 0.66667 | 0.00412 | 0.00355 | 0.00303 | 26 | 1.30081 |
| 0.10 | 1.40 | 89.69707 | 0.63636 | 0.66667 | 0.00422 | 0.00303 | 0.00261 | 28 | 1.30303 |
| 0.15 | 2.00 | 88.71336 | 0.62222 | 0.67925 | 0.00425 | 0.00295 | 0.00266 | 28 | 1.30147 |
| 0.14 | 1.90 | 89.69707 | 0.63043 | 0.67308 | 0.00431 | 0.00284 | 0.00247 | 29 | 1.30351 |
| 0.14 | 2.00 | 89.69707 | 0.63830 | 0.68627 | 0.00221 | 0.00137 | 0.00118 | 30 | 1.32457 |
| 0.32 | 1.77 | 138.34493 | 1.00000 | 0.30882 | 0.00027 | 0.00004 | 0.00002 | 30 | 1.30882 |
| 0.32 | 1.68 | 138.34493 | 1.00000 | 0.30882 | 0.00027 | 0.00004 | 0.00002 | 30 | 1.30882 |
| 0.32 | 1.76 | 138.34493 | 1.00000 | 0.30882 | 0.00027 | 0.00004 | 0.00002 | 30 | 1.30882 |
| 0.32 | 1.90 | 138.34493 | 1.00000 | 0.30882 | 0.00027 | 0.00004 | 0.00002 | 30 | 1.30882 |
| 0.32 | 1.78 | 138.34493 | 1.00000 | 0.30882 | 0.00027 | 0.00004 | 0.00002 | 30 | 1.30882 |
| 0.32 | 1.62 | 138.34493 | 1.00000 | 0.30882 | 0.00027 | 0.00004 | 0.00002 | 30 | 1.30882 |
| 0.16 | 2.01 | 102.29283 | 0.73810 | 0.57143 | 0.00373 | 0.00209 | 0.00195 | 31 | 1.30952 |
| 0.13 | 2.00 | 89.69707 | 0.62000 | 0.68750 | 0.00265 | 0.00218 | 0.00210 | 31 | 1.30750 |
| 0.16 | 2.14 | 102.29283 | 0.74419 | 0.58182 | 0.00194 | 0.00117 | 0.00106 | 32 | 1.32600 |
| 0.04 | 1.37 | 91.39885 | 0.64706 | 0.65957 | 0.00440 | 0.00292 | 0.00223 | 33 | 1.30663 |
| 0.10 | 2.00 | 89.69707 | 0.61818 | 0.72093 | 0.00109 | 0.00088 | 0.00072 | 34 | 1.33911 |
| 0.04 | 1.40 | 91.39885 | 0.65385 | 0.67391 | 0.00223 | 0.00135 | 0.00108 | 34 | 1.32776 |
| 0.10 | 2.01 | 89.69707 | 0.60714 | 0.71429 | 0.00212 | 0.00155 | 0.00141 | 34 | 1.32143 |
| 0.09 | 2.00 | 89.69707 | 0.60714 | 0.71429 | 0.00212 | 0.00155 | 0.00141 | 34 | 1.32143 |
| 0.15 | 2.14 | 102.29283 | 0.72340 | 0.58824 | 0.00233 | 0.00193 | 0.00170 | 34 | 1.31164 |
| 0.09 | 2.01 | 89.69707 | 0.59649 | 0.70732 | 0.00401 | 0.00293 | 0.00265 | 34 | 1.30381 |
| 0.08 | 2.00 | 89.69707 | 0.59649 | 0.70732 | 0.00401 | 0.00293 | 0.00265 | 34 | 1.30381 |
| 0.10 | 2.14 | 89.69707 | 0.61404 | 0.73171 | 0.00097 | 0.00062 | 0.00060 | 35 | 1.34574 |
| 0.14 | 2.01 | 102.29283 | 0.72917 | 0.60000 | 0.00121 | 0.00104 | 0.00091 | 35 | 1.32917 |
| 0.09 | 2.14 | 89.69707 | 0.60345 | 0.72500 | 0.00191 | 0.00122 | 0.00117 | 35 | 1.32845 |
| 0.08 | 2.14 | 89.69707 | 0.59322 | 0.71795 | 0.00363 | 0.00232 | 0.00222 | 35 | 1.31117 |
| 0.14 | 2.14 | 102.29283 | 0.73469 | 0.61224 | 0.00101 | 0.00054 | 0.00047 | 36 | 1.34694 |
| 0.13 | 2.01 | 102.29283 | 0.70588 | 0.59574 | 0.00414 | 0.00293 | 0.00246 | 36 | 1.30163 |
| 0.13 | 2.14 | 102.29283 | 0.71154 | 0.60870 | 0.00212 | 0.00162 | 0.00131 | 37 | 1.32023 |
| 0.06 | 2.00 | 91.39885 | 0.61667 | 0.68421 | 0.00667 | 0.00382 | 0.00340 | 37 | 1.30088 |
| 0.04 | 2.00 | 89.69707 | 0.57576 | 0.75000 | 0.00273 | 0.00213 | 0.00203 | 38 | 1.32576 |
| 0.05 | 2.00 | 91.39885 | 0.61290 | 0.69444 | 0.00607 | 0.00351 | 0.00305 | 38 | 1.30735 |
| 0.06 | 2.14 | 91.68645 | 0.62903 | 0.69444 | 0.00313 | 0.00202 | 0.00182 | 39 | 1.32348 |
| 0.05 | 2.01 | 91.68645 | 0.61905 | 0.68571 | 0.00583 | 0.00407 | 0.00353 | 39 | 1.30476 |
| 0.05 | 2.14 | 91.68645 | 0.62500 | 0.70588 | 0.00275 | 0.00185 | 0.00162 | 40 | 1.33088 |
| 0.04 | 2.01 | 91.68645 | 0.61194 | 0.70968 | 0.00447 | 0.00323 | 0.00274 | 41 | 1.32162 |
| 0.04 | 2.14 | 91.68645 | 0.61765 | 0.73333 | 0.00196 | 0.00161 | 0.00116 | 42 | 1.35098 |

In this table the top 50 out of 388 statistically significant combinations of MYC and MKLP2 cutoff values were shown. Data is sorted to maximize the number of true positives in the analysis. Sensitivity was measured as true positives/(true positives+false negatives). Specificity was measured as true negatives/(true negatives+false positives). The number of true positives were the (number of responsive cell lines in the MYC high and MKLP2 low group)/total number of cell lines.

TABLE 8
Statistical analysis of MYC and MKLP2 expression as
a predictive biomarker combination for LG157 IC50

| | | | | | | | | | Sum of |
|---|---|---|---|---|---|---|---|---|---|
| MYC | MKLP2 | Metric | | | | | | Number | Sensitivity |
| Cutoff | Cutoff | Cutoff | | | Fisher | Barnard | | of True | and |
| Value | Value | Value | Sensitivity | Specificity | p-value | p-value | Gtest | Positives | Specificity |
| 0.15 | 2.14 | 165.09862 | 0.70213 | 0.66667 | 0.00030 | 0.00024 | 0.00022 | 33 | 1.36879 |
| 0.14 | 2.01 | 165.09862 | 0.68750 | 0.66000 | 0.00067 | 0.00058 | 0.00051 | 33 | 1.34750 |
| 0.08 | 1.27 | 224.34564 | 0.80488 | 0.50877 | 0.00168 | 0.00141 | 0.00123 | 33 | 1.31365 |
| 0.15 | 2.01 | 165.09862 | 0.69565 | 0.65385 | 0.00064 | 0.00051 | 0.00048 | 32 | 1.34950 |
| 0.14 | 1.90 | 165.09862 | 0.69565 | 0.65385 | 0.00064 | 0.00051 | 0.00048 | 32 | 1.34950 |
| 0.14 | 2.00 | 165.09862 | 0.68085 | 0.64706 | 0.00136 | 0.00117 | 0.00106 | 32 | 1.32791 |
| 0.08 | 1.24 | 224.34564 | 0.80000 | 0.50000 | 0.00305 | 0.00252 | 0.00208 | 32 | 1.30000 |
| 0.15 | 1.90 | 165.09862 | 0.70455 | 0.64815 | 0.00060 | 0.00047 | 0.00044 | 31 | 1.35269 |
| 0.14 | 1.78 | 165.09862 | 0.68889 | 0.64151 | 0.00130 | 0.00104 | 0.00099 | 31 | 1.33040 |
| 0.15 | 2.00 | 165.09862 | 0.68889 | 0.64151 | 0.00130 | 0.00104 | 0.00099 | 31 | 1.33040 |
| 0.14 | 2.14 | 159.41478 | 0.63265 | 0.69388 | 0.00223 | 0.00120 | 0.00108 | 31 | 1.32653 |
| 0.13 | 2.14 | 153.73093 | 0.59615 | 0.71739 | 0.00232 | 0.00181 | 0.00165 | 31 | 1.31355 |
| 0.06 | 1.05 | 177.76453 | 0.73171 | 0.61404 | 0.00097 | 0.00063 | 0.00060 | 30 | 1.34574 |
| 0.06 | 1.02 | 177.76453 | 0.73171 | 0.61404 | 0.00097 | 0.00063 | 0.00060 | 30 | 1.34574 |
| 0.15 | 1.78 | 165.09862 | 0.69767 | 0.63636 | 0.00121 | 0.00098 | 0.00091 | 30 | 1.33404 |
| 0.16 | 2.14 | 165.09862 | 0.69767 | 0.63636 | 0.00121 | 0.00098 | 0.00091 | 30 | 1.33404 |
| 0.05 | 1.05 | 177.76453 | 0.71429 | 0.60714 | 0.00212 | 0.00155 | 0.00141 | 30 | 1.32143 |
| 0.06 | 1.08 | 177.76453 | 0.71429 | 0.60714 | 0.00212 | 0.00155 | 0.00141 | 30 | 1.32143 |
| 0.05 | 1.02 | 177.76453 | 0.71429 | 0.60714 | 0.00212 | 0.00155 | 0.00141 | 30 | 1.32143 |
| 0.06 | 1.06 | 177.76453 | 0.71429 | 0.60714 | 0.00212 | 0.00155 | 0.00141 | 30 | 1.32143 |
| 0.14 | 1.77 | 165.09862 | 0.68182 | 0.62963 | 0.00253 | 0.00216 | 0.00196 | 30 | 1.31145 |
| 0.14 | 1.68 | 165.09862 | 0.68182 | 0.62963 | 0.00253 | 0.00216 | 0.00196 | 30 | 1.31145 |
| 0.14 | 1.76 | 165.09862 | 0.68182 | 0.62963 | 0.00253 | 0.00216 | 0.00196 | 30 | 1.31145 |
| 0.14 | 1.62 | 165.09862 | 0.68182 | 0.62963 | 0.00253 | 0.00216 | 0.00196 | 30 | 1.31145 |
| 0.06 | 0.93 | 177.76453 | 0.72500 | 0.60345 | 0.00191 | 0.00123 | 0.00117 | 29 | 1.32845 |
| 0.09 | 1.27 | 177.76453 | 0.72500 | 0.60345 | 0.00191 | 0.00123 | 0.00117 | 29 | 1.32845 |
| 0.15 | 1.77 | 165.09862 | 0.69048 | 0.62500 | 0.00236 | 0.00193 | 0.00179 | 29 | 1.31548 |
| 0.15 | 1.68 | 165.09862 | 0.69048 | 0.62500 | 0.00236 | 0.00193 | 0.00179 | 29 | 1.31548 |
| 0.16 | 2.01 | 165.09862 | 0.69048 | 0.62500 | 0.00236 | 0.00193 | 0.00179 | 29 | 1.31548 |
| 0.15 | 1.76 | 165.09862 | 0.69048 | 0.62500 | 0.00236 | 0.00193 | 0.00179 | 29 | 1.31548 |
| 0.15 | 1.62 | 165.09862 | 0.69048 | 0.62500 | 0.00236 | 0.00193 | 0.00179 | 29 | 1.31548 |
| 0.09 | 1.31 | 177.76453 | 0.70732 | 0.59649 | 0.00401 | 0.00289 | 0.00265 | 29 | 1.30381 |
| 0.04 | 0.85 | 177.76453 | 0.70732 | 0.59649 | 0.00401 | 0.00289 | 0.00265 | 29 | 1.30381 |
| 0.05 | 0.93 | 177.76453 | 0.70732 | 0.59649 | 0.00401 | 0.00289 | 0.00265 | 29 | 1.30381 |
| 0.10 | 1.27 | 165.09862 | 0.71795 | 0.62712 | 0.00099 | 0.00079 | 0.00070 | 28 | 1.34507 |
| 0.04 | 0.80 | 177.76453 | 0.73684 | 0.60000 | 0.00169 | 0.00102 | 0.00095 | 28 | 1.33684 |
| 0.08 | 1.05 | 177.76453 | 0.73684 | 0.60000 | 0.00169 | 0.00102 | 0.00095 | 28 | 1.33684 |
| 0.09 | 1.05 | 177.76453 | 0.73684 | 0.60000 | 0.00169 | 0.00102 | 0.00095 | 28 | 1.33684 |
| 0.08 | 1.02 | 177.76453 | 0.73684 | 0.60000 | 0.00169 | 0.00102 | 0.00095 | 28 | 1.33684 |
| 0.09 | 1.02 | 177.76453 | 0.73684 | 0.60000 | 0.00169 | 0.00102 | 0.00095 | 28 | 1.33684 |
| 0.06 | 0.85 | 177.76453 | 0.73684 | 0.60000 | 0.00169 | 0.00102 | 0.00095 | 28 | 1.33684 |
| 0.08 | 0.93 | 177.76453 | 0.73684 | 0.60000 | 0.00169 | 0.00102 | 0.00095 | 28 | 1.33684 |
| 0.09 | 0.93 | 177.76453 | 0.73684 | 0.60000 | 0.00169 | 0.00102 | 0.00095 | 28 | 1.33684 |
| 0.18 | 2.01 | 165.09862 | 0.70000 | 0.62069 | 0.00216 | 0.00172 | 0.00160 | 28 | 1.32069 |
| 0.16 | 1.90 | 165.09862 | 0.70000 | 0.62069 | 0.00216 | 0.00172 | 0.00160 | 28 | 1.32069 |
| 0.10 | 1.31 | 165.09862 | 0.70000 | 0.62069 | 0.00216 | 0.00172 | 0.00160 | 28 | 1.32069 |
| 0.18 | 2.14 | 165.09862 | 0.70000 | 0.62069 | 0.00216 | 0.00172 | 0.00160 | 28 | 1.32069 |
| 0.07 | 1.05 | 177.76453 | 0.71795 | 0.59322 | 0.00363 | 0.00233 | 0.00222 | 28 | 1.31117 |
| 0.09 | 1.24 | 177.76453 | 0.71795 | 0.59322 | 0.00363 | 0.00233 | 0.00222 | 28 | 1.31117 |
| 0.08 | 1.08 | 177.76453 | 0.71795 | 0.59322 | 0.00363 | 0.00233 | 0.00222 | 28 | 1.31117 |

In this table the top 50 out of 388 statistically significant combinations of MYC and MKLP2 cutoff values were shown. Data is sorted to maximize the number of true positives in the analysis. Sensitivity was measured as true positives/(true positives+false negatives). Specificity was measured as true negatives/(true negatives+false positives). The number of true positives were the (number of responsive cell lines in the MYC high and MKLP2 low group)/total number of cell lines.

### Results

### Assay Development

Datasets to examine the MYC- and MKLP2-dependence of compounds were performed with the below-referenced steps, and repeated with a diverse set of compounds.

In a first step, a panel of 98 human cancer cell lines of various origins were selected, including lung (22), breast (18), blood (13), brain (8), ovary (6), skin (6), intestine (5), pancreas (4), bone (3), kidney (3), prostate (3), stomach (3), cervix (2), and liver (2).

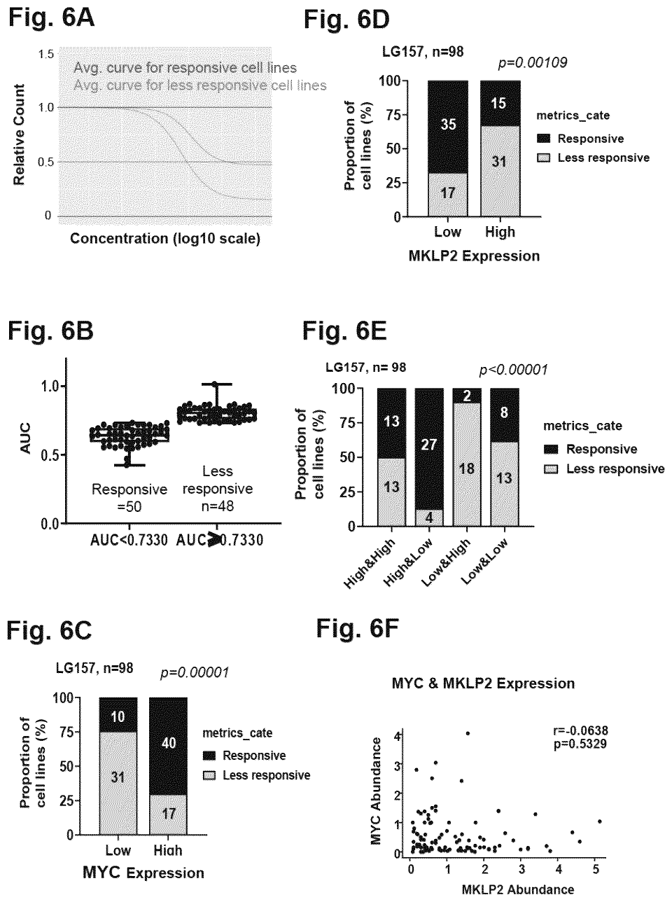

In a second step, a quantitative immunoblot blot analysis of MYC and MKLP2 expression using the Li-COR NIR fluorescence detection system was performed. The protein levels of MYC, MYCN, and MYCL in all 98 lines of the cancer cell line panel were quantified (FIGS. 5A-5D). Similarly, MKLP2 was quantified in the panel of cell lines (FIG. 6F). Combined with concentration-response data, the expression data revealed to examine the effect of compounds at varying thresholds of MYC and/or MKLP2 expression. The systematic approach enabled cell lines to be divided into MYC^{High}/MYC^{Low} and/or MKLP2^{High}/MKLP2^{Low} responsivity groups (FIG. 6A, FIGS. 6C-6E), that maximized sensitivity, specificity, and p values of the statistical comparisons.

In a third step, an analysis was conducted using the genotype of select cancer genes. The mutational and copy number landscape of 89 of a panel of cancer cell lines was obtained from The Cancer Genome Project. Examples of genes reviewed included Ras, Raf, p53, and RB.

In a fourth step, gene-dependence data for MKLP2 and other mitotic regulators was obtained from the Cancer Dependency Map Project for 58 cell lines in a panel of 98. The cell fitness data after CRISPR-mediated deletion of MKLP2 and other mitotic regulators of interest in the 58 cell lines were subjected to correlation analysis with MYC abundance.

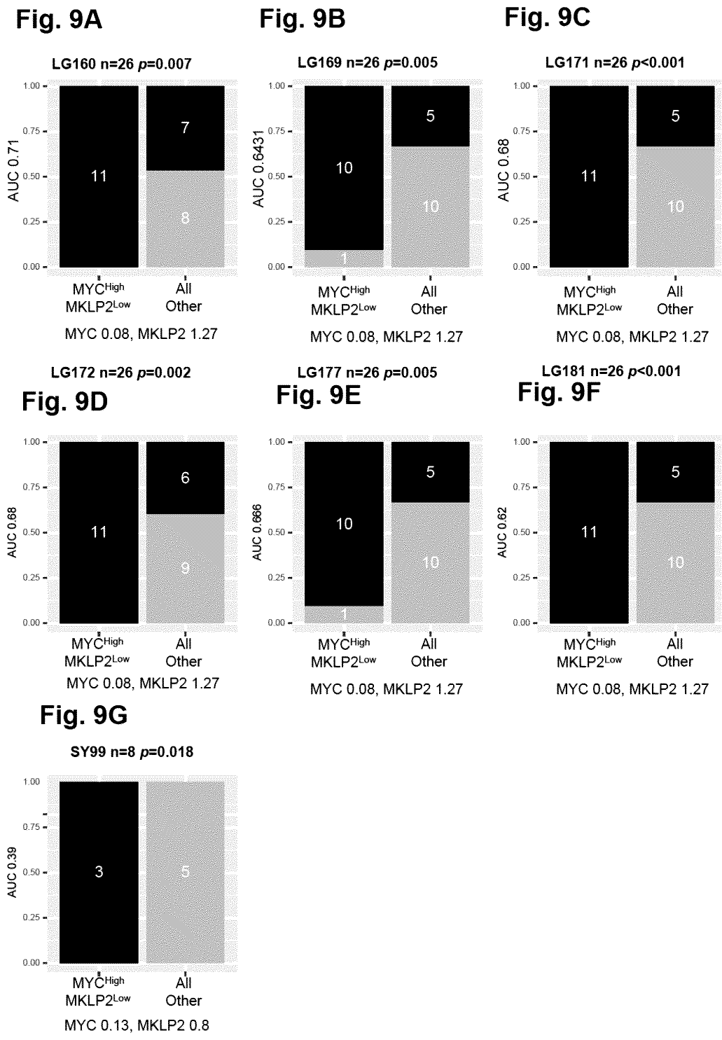

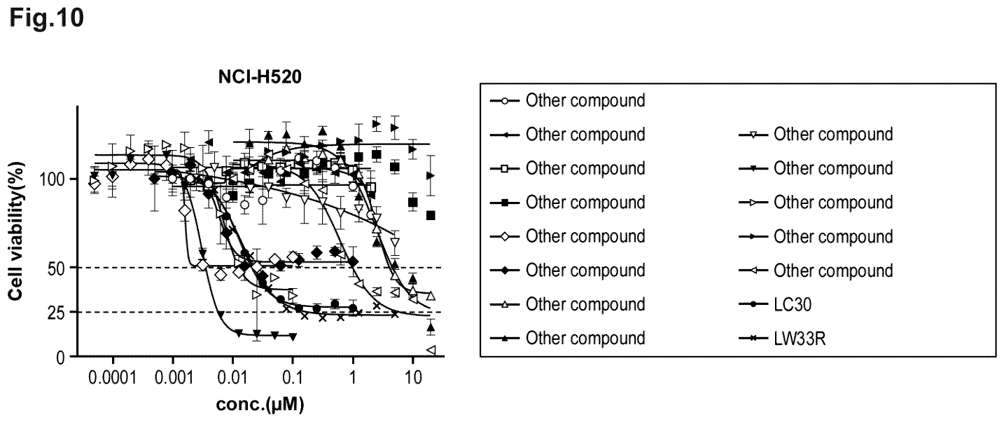

In a fifth step, concentration-response experiments were carried out in all 98 cell lines, which involved analysis of the slope of the dose-response curve (h), IC₅₀, half-maximal response concentration (EC₅₀), the maximum effect (Eₘₐₓ), and the area under the curve (AUC; FIG. 6B and FIGS. 8A-8G). Similar analyses were conducted in a limited set of the 98 cell lines (FIGS. 9A-9G). IC₅₀ and EC₅₀ are descriptors for drug potency without reflecting an effect size, whereas Eₘₐₓ is a parameter for efficacy. AUC combines potency and efficacy into a single parameter, which can be used to compare a single drug across multiple cell lines exposed to the same range of drug concentrations. After generation of dose-response curves for MYC-synthetic lethal (SL) treatment compounds in a panel of 98 cancer cell lines (FIG. 5E and FIGS. 10-21E), the correlation between the MYC and/or MKLP2 abundance and each of the parameters described above was analyzed.

### Demonstration of MYC's Predictive Value: The Activity of LG157 in a Model Cell Screen

In a drug screening, LG157 was shown to be active in a pair of model cell lines, RPE-NEO and RPE-MYC. In the model screening cell lines, there was an indistinguishable threshold concentration of approximately 20 nM, which was required to show disruption of the CPPC complex by IHC at the spindle midzone and equatorial cortex of anaphase cells, indicating a failure in CPPC relocation. This similarity in the threshold effective concentration for target inhibition is, therefore, not influenced by overexpression of MYC. It was also observed that extensive cell death occurred in RPE-MYC but not RPE-NEO cells, despite comparable levels of disruption of the CPPC complex localization in both lines. The results of this selective toxicity demonstrates that inhibition of MKLP2 is not insufficient, by itself, to trigger cell death in normal cells and that overexpressed MYC primes cells to the lethal effect of LG157. Similar data with other MYC-SL compounds were observed.

### Elevated MYC Predicts Acute Cytotoxicity

In the analysis of 98 cell lines treated with LG157, MYC abundance was positively correlated with Eₘₐₓ value and was negatively correlated with IC₅₀, EC₅₀, and AUC. MYC lacked a correlation with the h value. Similar results were obtained when multiple cutoffs for MYC expression were examined. IC₅₀ values differentiated the MYC^{Low} group from the MYC^{High} group more effectively than EC₅₀, as evidenced by a greater significant p-value, as derived from Fisher's exact test. AUC was used for detailed analysis, as partitioning the AUC values at 0.7330 maximized sensitivity, specificity, and p values of the Fisher's exact test (Table 6, for optimal cutoff, the metric was AUC (e.g., values of AUC/EC50/Emax/IC50 that maximize the sum of sensitivity and specificity at a given MYC and MKLP2 value)), the compound tested was LG157, the biomarker combination tested was CMYC/MKLP2). This placed 80% of the responsive cell lines in the MYC^{High} group (Table 3). Taken together, this data demonstrates that for LG157, the abundance of MYC in the human cancer cell lines positively relates to sensitivity.

The same conclusions were reached when cell lines with only abundant MYCN or MYCL were included or excluded in the MYC^{High} group. This preferential killing of MYC^{High} cells was particularly evident when lung cancer cell lines were analyzed separately.

In studies at the drug concentrations that elicited Eₘₐₓ, cytokinesis was blocked in all cell lines. This result was observed irrespective of MYC abundance. Therefore, the diminished acute cytotoxicity of MYC-SL compounds in MYC^{Low} cell lines could not be attributed to the failure of target inhibition. This finding matches the observations in RPE-MYC versus RPE-NEO cells.

### Enhanced Predictive Value Through Combined MYC/MKLP2 Assessment

In contrast to MYC, it was found that lower protein levels of MKLP2, but not high, predicted a favorable response to MYC-SL compounds, such as LG-157 (FIG. 6D). The predictive value of having low MKLP2 was not as strong of a predictor of which tumors would be susceptible to MYC-SL drugs, as was the prediction provided by elevated levels of MYC. Parsing the dataset on both MYC and MKLP2 lends further predictive ability. Tumors that were MYC^{High} and MKLP2^{Low} were most often found in the group having a favorable drug response (FIG. 6E). Conversely, the MYC^{Low} and MKLP2^{High} tumors were found significantly to be in the less responsive group.

A correlation between MYC and MKLP2 expression might occur if the MKLP2 gene were a target of MYC, though the results demonstrated that no correlation between MYC and MKLP2 protein abundance was observed, thereby demonstrating that MKLP2 is likely not be transcriptionally regulated by MYC (FIG. 6F). It was also observed that some cancer cell lines have MKLP2 protein levels lower than proliferating normal cells. This data demonstrate that the possibility exists to inhibit MKLP2 in cancer cells to induce an antitumor effect, while the same level of MKLP2 inhibition will not be detrimental to normal tissues.

Cell Death with MKLP2 Gene Deletion and MYC

Data from the Cancer Dependency Map Project was used to see if lethality from MKLP2 gene deletion using the CRISPR/Cas9 system was related to MYC overexpression. MKLP2 gene deletion decreased viability in 700 out of 1070 human cancer cell lines in the database. Data was present for 58 of our cell lines. In this subpanel of cell lines, high MYC protein level correlated with a negative effect on cell fitness elicited by genetic deletion of MKLP2, thereby establishing the line of evidence that MYC primes cells for death with the loss of MKLP2. In contrast with the findings with MKLP2, in this subpanel of 58 cell lines, MYC failed to modify the fitness of cells with CRISPR/Cas9-mediated deletion of other mitotic regulators, including PLK1, PLK4, EG5/KSP-cadherin, AURKA, CENP-E, Mad1, Mad2, Bub1, and BubR1. These results reveal that the loss of MKLP2, but not other mitotic regulators, exacerbates mitotic defects enabled by overexpression of MYC, resulting in synthetic lethality.

### Other Oncogenic Drivers Fail to Modify Sensitivity to MYC-SL Compounds

Genomic data cataloging oncogenic mutations and copy number alterations in cells of the panel of human cancer lines were obtained from publicly available datasets. The status of the tumor suppressor genes TP53, RB, and PTEN, as well the presence of activating mutations for oncogenes RAS, BRAF, and PI3K, were available for 89 of the 98 cell lines. A correlation analysis was performed between drug response and these oncogenic alterations. None of these alterations predicted a favorable response to the MYC-SL compounds (FIGS. 7A-7F). Therefore, no evidence was found that these alterations were associated with either sensitivity or resistance to the MYC-SL compound LG157.

### CONCLUSIONS

Taken together, these results demonstrate the development of a methodology for testing synthetic lethality (SL) in a large panel of cell lines that can be applied to compounds in subsequent stages of drug development. MYC and MKLP2 were demonstrated to be positive and negative biomarkers, respectively, for loss of cell viability through the induction of mitotic catastrophe by the tested drug candidates.

MYC-SL compounds also elicit a distinct MYC-dependent phenotype, including cell viability and suppression of cytokinesis. In a Myc^{Low} cell line, compounds elicited cytokinetic failure, even without the induction of extensive cell death. The inhibition of cytokinesis should suppress proliferation, thereby effecting the number of viable cells. In line with this postulate, in a Myc^{High} cell line, MYC-SL compounds not only triggered cell death but also blocked cytokinesis. It was demonstrated that the combined effects on cell proliferation and viability act synergistically.

Some of the agents tested herein reproducibly induced these phenotypes in a dose-dependent manner, without inhibiting the kinase activity of either AURKA or AURKB, as assayed through surrogate phosphorylation events mediated by these kinases. In anaphase cells exposed to one of the agents herein, AURKB and INCENP remain on mitotic chromosomes in dividing cells and are absent from the spindle midzone and equatorial cortex, indicating a failure in relocation. The threshold concentrations required to block cytokinesis, as indicated by the accumulation of polyploid cells, was consistent with that required to prevent the relocation of the CPPC complex. This finding demonstrates that a class of compounds inhibits localization of mitotic regulator proteins, which, in turn, causes a failure in cytokinesis.

Based on these observations, it is expected that the agents herein, which preferentially target MYC overexpressing cells, could target upstream regulators of the CPPC. Since MKLP2 is an upstream regulator of the CPPC complex, it may be a determinant of sensitivity. As a combination, MYC and MKLP2 serve as biomarkers for a diverse group of compounds, including those that mis-localize mitotic complexes and those that are direct catalytic inhibitors of mitotic kinases.

There are several advantages inherent to the approach taken herein to validate the correlation of the agents that inhibit cancer cell viability and/or proliferation (e.g., LG157) with levels of MYC and MKLP2 expression. First, the human cell lines in the panel have a variety of protein expression levels, such that not only MYC^{High} tumors were tested. The number of cell lines used and varying expression levels enables statistically meaningful thresholds of expression. Second, the availability of genomic data on these cell lines through The Cancer Genome Atlas (TCGA), or other sources, enabled combined analyses with other genomic events, confirming the above described effect enabled by the above-discovered cancer biomarkers: MYC and MKLP2.

### Example 2: A Method of Treating Cancer

Using the methods of the disclosure, an individual diagnosed with cancer is treated with a therapeutically effective amount of a one or more agent(s) that inhibit cancer cell viability and/or proliferation disclosed herein. Such treatment alleviates one or more symptoms of the individual's symptoms of cancer.

### Example 3: A Method of Preventing Cancer

Using the methods of the disclosure, an individual at risk of developing cancer is administered an effective amount of a one or more agent(s) that inhibit cancer cell viability and/or proliferation disclosed herein. Such administration serves as a prophylactic treatment for one or more symptoms of cancer or for the development of cancer.

### Example 4: A Method of Treating Cancer

Using the methods of the disclosure, an individual at risk of developing cancer, suffering from one or more (e.g., two, three, or four) symptoms associated with cancer, or diagnosed with cancer is treated with one or more (e.g., two, three, or four) agent(s) that inhibit cancer cell viability and/or proliferation disclosed herein.

For example, a biological sample (e.g., tissue, plasma, blood, stool, urine, or combinations thereof) is obtained from the individual suspected of being at risk of developing cancer, suffering from one or more symptoms associated with cancer, or diagnosed with cancer; a cell obtained from the biological sample is processed to produce a test cell; the test cell is contacted with a one or more agent(s) that inhibit cancer cell viability and/or proliferation to produce a one or more agent(s) that inhibit cancer cell viability and/or proliferation-induced response; the expression and/or activity of one or more (e.g., two, three, or four) genes of the cancer-dependent gene signature (e.g., MYC and/or MKLP2) is detected; an individual as at risk of developing cancer is identified or the individual with cancer is diagnosed when the expression or activity of the one or more (genes of the cancer-dependent gene signature are modified relative to a suitable control (e.g., a control with substantially no test agent); and the individual identified as at risk of developing cancer or diagnosed with cancer is administered the one or more agent(s) that inhibit a cancer cell viability and/or proliferation response.

In another example, an individual at risk for developing cancer, diagnosed with cancer, or experiencing one or more symptoms associated with cancer, is administered one more agent(s) that inhibit cancer cell viability and/or proliferation. Such a method serves as a method of treatment.

### Example 5: Identifying Individuals at Risk of Developing Cancer

Using the methods of the disclosure, an individual at risk of developing cancer or diagnosed with cancer is identified. For instance, a dataset including data associated with expression and/or activity of MYC and MKLP2 in a biological sample obtained from the individual is obtained; and the individual is identified as at risk of developing cancer or having cancer when the expression and/or activity of MYC is increased and the expression and/or activity of MKLP2 is decreased relative to a suitable control. Alternatively, for example, the a biological sample (e.g., tissue, plasma, blood, stool, or urine) is obtained from the individual suspected of being at risk of developing cancer or diagnosed with cancer, a cell obtained from the biological sample is processed by contacting the test cell with a one or more agent(s) that inhibit cancer cell viability and/or proliferation to produce a cancer cell viability and/or proliferation-induced response, the expression and/or activity of the one or more (e.g., two, three, or four) genes of the cancer-dependent gene signature (e.g., MYC and/or MKLP2) is detected, and an individual is identified as having an elevated risk for cancer or the individual is diagnosed with cancer if the expression and/or activity of the one or more genes of the cancer-dependent gene signature are elevated (e.g., MYC) or decreased (e.g., MKLP2) relative to a suitable control (e.g., a control with substantially no test agent). Alternatively, for example, an individual is identified as not having an elevated risk for cancer or not having cancer if the expression and/or activity of the one or more genes of the cancer-dependent gene signature are not found to be elevated (e.g., MYC) or decreased (e.g., MKLP2) relative to a suitable control (e.g., a control with substantially no test agent).

### Example 6: A Method of Screening Compounds for Treating Cancer

Using the methods of the disclosure, compounds are screened for their ability to reduce the risk of an individual developing cancer, reduce the risk of an individual developing one or more symptoms of cancer, or alleviate one or more symptoms of cancer. For example, cancer cells or a sample derived from cancer cells are contacted with one or more test agents; (ii) the expression and/or activity of MYC and MKLP2 in the cancer cells is detected; and (iii) if the test agent modifies a cancer-dependent signature or MYC-dependent cellular phenotypic signature, the test agent is identified as a compound effective for the treatment of cancer.

### Example 7: A Method for Monitoring Cancer Status

Cancer status (e.g., progression or regression) is measured during therapy using the methods of the disclosure. In order to monitor the status of cancer in an individual, individual samples are compared to reference samples taken early in the diagnosis of the disorder. Such monitoring can be useful, for example, in assessing the efficacy of a particular therapeutic agent (e.g., a one or more agent(s) that inhibit cancer cell viability and/or proliferation) in an individual, determining dosages, or in assessing disease progression or status. For example, the expression and/or activity of any of the genes described herein (e.g., MYC and/or MKLP2) are monitored in an individual, and as the expression levels or activities increase or decrease, relative to control, the dosage or administration of therapeutic agents are adjusted. For example, modifications (e.g., an increase or a decrease as compared to a prior sample of an individual) are detected to indicate an improvement or decline in cancer status. For example, the levels of the cancer-dependent gene signature are measured repeatedly as a method of monitoring the treatment, prevention, or management of the disorder.

### Example 8: A Method for Optimizing the Dosage of a Compound for the Treatment of Cancer

Using the methods of the disclosure, the proper dosage (e.g., the therapeutically effective amount) of a therapeutic agent for an individual, the proper duration of dosage of a therapeutic agent for an individual, the proper type of therapeutic agent, or whether a therapy should be administered is determined. For example, cells (e.g., fibroblasts, neurons, or blood cells) are obtained from individuals at risk of developing cancer or suffering from cancer, a cell obtained from the biological sample is processed to produce a test cell, the cells (e.g., fibroblasts, neurons, or blood cells) are contacted with one or more test agents, the expression and/or activity of one or more (e.g., two, three, or four) genes of the cancer-dependent gene signature (e.g., MYC and/or MKLP2) is detected, and the proper dosage or dosage duration of a test agent by assessing modifications to the transcriptional profile of the cancer-dependent gene signature (e.g., MYC and/or MKLP2) is identified.

### Example 9. Synthesis of LG181

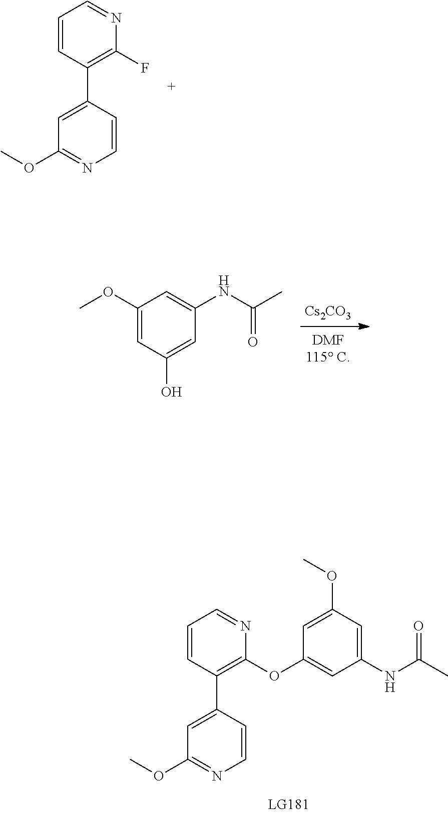

N-(3-methoxy-5-((2′-methoxy-[3,4′-bipyridin]-2-yl)oxy)phenyl) acetamide (LG181): 2-fluoro-2′-methoxy-3,4′-bipyridine (102 mg, 0.5 mmol, 1.0 eq), N-(3-hydroxy-5-methoxyphenyl) acetamide (91 mg, 0.5 mmol, 1 eq) and Cs₂CO₃ (326 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (200 mg, yield=99%, purity=99%)

TLC R_{f}=0.2 (PE/EA=1/2)

MS (ESI⁺): m/z=366.50 (M+1)

¹H NMR: (400 MHz, DMSO-d₆) δ 9.99 (s, 1H), 8.26 (d, J=5.4 Hz, 1H), 8.22 (dd, J=4.8, 1.9 Hz, 1H), 8.03 (dd, J=7.5, 1.9 Hz, 1H), 7.33-7.23 (m, 2H), 7.11 (d, J=1.4 Hz, 1H), 7.05 (t, J=2.0 Hz, 1H), 6.98 (t, J=1.9 Hz, 1H), 6.46 (t, J=2.2 Hz, 1H), 3.89 (s, 3H), 2.02 (s, 3H). ¹³C NMR: (101 MHz, DMSO) δ 168.94, 164.33, 160.81, 159.76, 155.55, 148.23, 147.38, 146.72, 141.38, 140.60, 122.78, 120.31, 117.78, 110.75, 104.59, 102.27, 101.42, 55.74, 55.37, 53.69, 24.56.

### INCORPORATION BY REFERENCE

The entire disclosure of each of the patent documents and scientific articles cited herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A method of treating an individual at risk of developing cancer, suffering from one or more symptoms associated with cancer, and/or diagnosed with cancer, comprising:
obtaining a dataset comprising data associated with expression and/or activity of MYC and MKLP2 in a biological sample obtained from the individual; and
identifying the individual as at risk of developing cancer or having cancer when the expression and/or activity of MYC is increased and the expression and/or activity of MKLP2 is decreased relative to a suitable control.

2. The method of claim 1, further comprising obtaining the biological sample from the individual, and optionally further comprising processing the sample to produce the dataset.

3. The method of claim 1 or 2, wherein the biological sample comprises a tissue, plasma, blood, stool, urine, or combinations thereof.

4. The method of claim 3, wherein the biological sample is obtained from a tissue biopsy, aspirate, or surgical removal.

5. The method of any one of claims 1-4, further comprising administering to the individual one or more agent(s) that inhibit cancer cell viability and/or proliferation.

6. A method of treating a cancer with increased expression and/or activity of MYC and decreased expression and/or activity of MKLP2 in an individual, the method comprising administering to the individual one more agent(s) that inhibit cancer cell viability and/or proliferation.

7. The method of claim 5 or 6, wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of the chromosomal passenger protein complex (CPPC).

8. The method of any one of claims 5-7, wherein the one or more agent(s) that inhibit cancer cell viability and/or proliferation comprises an agent selected from LC30, LW33R, LXY013, LXY018, LC02, LC09, LG157, LG160, LG169, LG171, LG172, LG177, and LG181.

9. A method for identifying an individual who may benefit from a treatment comprising one or more agent(s) that inhibit cancer cell viability and/or proliferation, comprising: obtaining a dataset associated with expression and/or activity of MYC in a biological sample obtained from the individual;
wherein an expression and/or activity of MYC that is elevated relative to a suitable control identifies the individual as one who may benefit from a treatment comprising one or more agent(s) that inhibit cancer cell viability and/or proliferation; and
wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC.

10. The method of claim 9, further comprising obtaining the biological sample from the individual, and optionally further comprising processing the sample to produce the dataset.

11. The method of claim 9 or 10, wherein the agent that inhibits cancer cell viability, comprises an agent selected from LC30, LW33R, LXY013, LXY018, LC02, LC09, LG157, LG160, LG169, LG171, LG172, LG177, and LG181.

12. A method for identifying an individual who may benefit from a treatment comprising one or more agent(s) that inhibit cancer cell viability and/or proliferation, comprising: obtaining a dataset comprising data associated with expression and/or activity of MKLP2 in biological sample obtained from the individual;
wherein an expression and/or activity of MKLP2 that is decreased relative to a suitable control identifies the individual as one who may benefit from a treatment comprising one or more agent(s) that inhibit cell viability; and
wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC.

13. The method of claim 14, further comprising obtaining the biological sample from the individual, and optionally further comprising processing the sample to produce the dataset.

14. The method claim 12 or 13, wherein the one or more agent(s) that inhibit cancer cell viability and/or proliferation comprises an agent selected from LC30, LW33R, LXY013, LXY018, LC02, LC09, LG157, LG160, LG169, LG171, LG172, LG177, and LG181.

15. A method for identifying an individual who may benefit from a treatment comprising one or more agent(s) that inhibit cancer cell viability and/or proliferation, comprising:
obtaining a dataset comprising data associated with expression and/or activity of MYC and MKLP2 in a biological sample obtained from the individual;
wherein an expression and/or activity of MYC that is elevated relative to a suitable control and an expression and/or activity of MKLP2 that is decreased relative to a suitable control identifies an individual as one who may benefit from a treatment comprising one or more agent(s) that inhibit cancer cell viability and/or proliferation.

16. The method of claim 15, further comprising obtaining the biological sample from the individual, and optionally further comprising processing the sample to produce the dataset.

17. The method of claim 15 or 16, wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC.

18. The method of any one of claims 15-17, wherein the one or more agent(s) that inhibit cancer cell viability and/or proliferation comprises an agent selected from LC30, LW33R, LXY013, LXY018, LC02, LC09, LG157, LG160, LG169, LG171, LG172, LG177, and LG181.

19. A method of screening compounds for the treatment of cancer, comprising:
i) contacting cancer cells or a sample derived from cancer cells with one or more test agents;
ii) detecting the expression and/or activity of MYC and MKLP2 in the cancer cells; and
iii) if the test agent modifies a cancer-dependent gene signature or MYC-dependent cellular phenotypic signature, identifying the test agent as a compound effective for the treatment of cancer.

20. The method of claim 19, wherein the cancer cells are cancer-derived cells, immortalized cells, or primary cells.

21. The method of any one of claims 1-20, wherein the detecting comprises detecting MYC and/or MKLP protein.

22. The method of claim 21, wherein the detecting comprises performing an immunofluorescence assay, an immunoblot, a proteomic analysis, or an ELISA on the biological sample or cancer cells.

23. The method of any one of claims 1-22, wherein the detecting comprises detecting MYC and/or MKLP2 RNA.

24. The method of claim 23, wherein detecting comprises performing RNA sequencing, a Taqman assay, or fluorescence in situ hybridization (FISH) on the biological sample or cancer cells.

25. The method of any one of claims 1-24, wherein the detecting comprises detecting an abnormality in the MYC gene and/or the MKLP2 gene.

26. The method of claim 25, wherein the detecting the abnormality in the MYC gene, comprises detecting a mutation in MYC, a translocation of MYC, a copy number of MYC, or combinations thereof.

27. The method of claim 26, wherein the detecting the abnormality in MKLP2 gene comprises detecting a mutation in MKLP2, a copy number of MKLP2, a copy number of MKLP2, or combinations thereof.

28. The method of any one of claims 1-27, wherein the detecting MYC expression and/or activity comprises detecting MYCN and/or MYCL.

29. The method of any one of claims 1-28, wherein the detecting comprises detecting a transcriptional profile of a cancer-dependent gene signature and/or detecting a MYC-dependent cellular phenotypic signature.

30. The method of claim 29, wherein detecting the transcriptional profile of a cancer-dependent gene signature comprises detecting expression of MYC and MKLP2, wherein detecting expression of MYC and MKLP2 comprises sequencing RNA derived from the biological sample or cancer cells.

31. The method of claim 29, wherein detecting the MYC-dependent cellular phenotypic signature comprises performing an image-based screening assay, a 3-(4,5-dimethylthiazol-2-yl)-2,5-diphenyltetrazolium bromide tetrazolium (MTT) assay, a CellTiter-Glo® (CTG) assay, a lactate dehydrogenase (LDH) assay, or combinations thereof.

32. The method of claim 31, wherein the image-based screening assay comprises detection of mitotic arrest, detection of induction of polyploidy, detection of cell death, an immunofluorescent assay, or combinations thereof.

33. A method of detecting expression and/or activity of MYC and MKLP2, comprising:
contacting a biological sample obtained from an individual diagnosed with cancer or experiencing one or more symptoms associated with cancer with at least a first and second test agent;
wherein the first test agent detects MYC expression and/or activity and the second test agent detects MKLP2 expression and/or activity.

34. The method of claim 33, wherein the method further comprises detecting expression of MYC and/or MKLP2.

35. The method of claim 32 or 33, wherein the first test agent comprises an antibody that binds MYC.

36. The method of any one of claims 33-35, wherein the second test agent comprises an antibody that binds MKLP2.

37. The method of any one of claims 34-36, wherein expression of MYC and/or MKLP2 is quantified.

38. The method of claim 37, wherein the quantification comprises quantification of an image obtained from an immunohistochemistry assay, a FISH assay, an RNA-seq, a Taqman, quantitative PCR, proteomics assay, an immunoblot, or an ELISA.

39. The method of claim 37 or 38, wherein the individual is identified as an individual who may benefit from a treatment comprising one or more agent(s) that inhibit cancer cell viability and/or proliferation if expression of MYC is elevated relative to a suitable control and expression of MKLP2 is decreased relative to a suitable control.

40. The method of claim 39, wherein the suitable control comprises expression of β-Actin in the same biological sample.

41. The method of any one of claims 1-40, wherein the biological sample comprises lung cells, breast cells, blood cells, brain cells, ovary cells, skin cells, intestine cells, pancreas cells, bone cells, kidney cells, prostate cells, stomach cells, cervix cells, or liver cells.

42. The method of any one of claims 1-41, wherein the biological sample comprises biopsied tissue.

43. The method of claim 42, wherein the biopsied tissue comprises biopsied tumor tissue.

44. The method of any one of claims 1-43, wherein MYC comprises the MYC protein family, wherein the MYC protein family comprises one or more of MYC, MYCN and MYCL.

45. The method of any one of claims 1-44, wherein the cancer is bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, soft tissue sarcoma, melanoma, astrocytoma, oligoastrocytoma, bone cancer, brain cancer, gastrointestinal cancer, cardiac cancer, uterine cancer, head and neck cancer, gallbladder cancer, laryngeal cancer, lip and oral cavity cancer, ocular cancer, colorectal cancer, testicular cancer, throat cancer, acute lymphoblastic leukemia (ALL), chronic myelogenous leukemia (CML), adrenocortical carcinoma, AIDS-related lymphoma, primary CNS lymphoma, anal cancer, appendix cancer, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, extrahepatic ductal carcinoma in situ (DCIS), endometrial cancer, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, myelodysplastic syndromes, nasal cavity and paranasal sinus cancer, nasopharyngeal cancer, epithelial ovarian cancer, germ cell ovarian cancer, low malignant potential ovarian cancer, papillomatosis, paraganglioma, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, sezary syndrome, small intestine cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or waldenström macroglobulinemia.

46. The method of any one of claims 1-45, wherein the suitable control is a biological sample without cancer.

47. The method of claim 46, wherein the suitable control is a biological sample without cancer from the individual.

48. The method of any one of claims 1-46, wherein the suitable control is a predetermined threshold determined from a biological sample obtained from individuals or tissues without cancer.

49. The method of any one of claims 39-48, wherein the one or more agent(s) that inhibit cancer cell viability and/or proliferation comprises an agent that dysregulates MYC, one or more mitotic kinase(s), one or more mitotic motor protein(s), and/or an upstream regulator or a downstream effector of CPPC.

50. The method of claim 49, wherein the one or more agent(s) that inhibit cancer cell viability and/or proliferation is selected from LC30, LW33R, LXY013, LXY018, LC02, LC09, LG157, LG160, LG169, LG171, LG172, LG177, and LG181.

51. A kit for detecting expression of MYC and MKLP2 in a biological sample obtained from an individual who may benefit from a treatment comprising one or more agent(s) that inhibit cancer cell viability and/or proliferation, the kit comprising:
(i) at least a first and a second test agent, wherein the first test agent detects MYC expression and the second test agent detects MKLP2 expression; and
(ii) instructions for use.
